(19) United States
(12) Patent Application Publication
(10) Pub. No.: US 2020/0079766 A1
(43) Pub. Date: Mar. 12, 2020

(54) MONOCYCLIC OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Carlos Manuel Martinez Viturro, Olias del Rey (ES); Gary John Tresadem, Toledo (ES); Manuel Jesús Alcázar-Vaca, Toledo (ES); Wei Zhang, San Diego, CA (US); Gang Chen, San Diego, CA (US)

(21) Appl. No.: 16/469,701

(22) PCT Filed: Dec. 15, 2017
(86) PCT No.: PCT/EP2017/083136

§ 371 Date: Jun. 14, 2019

(30) Foreign Application Priority Data
Dec. 16, 2016 (EP) 16204840.9

(51) Int. Cl. C07D 417/14; C07D 417/06; C07D 401/14; C07D 405/14; C07D 413/14

(52) U.S. Cl. CPC C07D 417/14; C07D 417/06; C07D 413/14; C07D 405/14; C07D 401/14

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

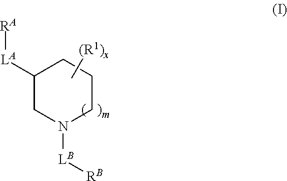

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in then-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2008/012623 (Pfizer Prod. Inc., published 31 Jan. 2008) discloses 2-[(4-phenyl-1-piperidyl)methyl]-1H-benzimidazole and 2-[(3-phenylpyrrolidin-1-yl)methyl]-1H-benzimidazole derivatives and as an exception, 2-(3-benzylpyrrolidin-1-yl)methyl]-1H-benzimidazole as mGluR2 potentiators.

WO2007/115077 (AstraZeneca A.B. and NPS Pharma Inc., published 11 Oct. 2007) discloses mainly 1H-benzimidazol-2-ylmethyl substituted 4-piperidines and 3-pyrrolidines, bearing at the 4- or 3-position respectively a phenylalkyl substituent, such as for example, 2-[3-(4-fluorobenzyl)-piperidin-1-ylmethyl]-1-methyl-1H-benzoimidazole, as mGluR potentiators.

WO03/092678 (Schering AG, published 13 Nov. 2007) describes substituted imidazole derivatives as NOS inhibitors, and describes (3S)-3-(4-aminophenoxy)-1-[(1,3-benzodioxol-5-yl)methyl]piperidine as an intermediate of synthesis.

WO93/21181 (Merck Sharp & Dohme, published 28 Oct. 1993) discloses Tachykinin antagonists. Particular example 6, 2-[{(2R*,3R*)-3-((3,5-bis(trifluoromethyl)phenyl)methyloxy)-2-phenylpiperidino}methyl]benzimidazole, requires a phenyl substituent at the piperidine.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors.

WO2014/159234 (Merck Patent GMBH, published 2 Oct. 2014) discloses mainly 4-phenyl or benzyl-piperidine and piperazine compounds substituted at the 1-position with an acetamido-thiazolylmethyl or acetamidoxazolylmethyl substituent and the compound N-[5-[(3-phenyl-1-piperidyl)methyl]thiazol-2-yl]acetamide;

WO2016/0300443 (Asceneuron S.A., published 3 Mar. 2016), WO2017/144633 and

WO2017/0114639 (Asceneuron S.A., published 31 Aug. 2017) disclose 1,4-disubstituted piperidines or piperazines as OGA inhibitors;

WO2017/144637 (Asceneuron S.A, published 31 Aug. 2017.) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors;

WO2017/106254 (Merck Sharp & Dohme Corp.) describes substituted N-[5-[(4-methylene-1-piperidyl)methyl]thiazol-2-yl]acetamide compounds as OGA inhibitors.

The following compounds are commercially available:
2-[1-[(2,3-dihydro-1,4-benzodioxin-6-yl)methyl]-3-piperidinyl]-pyrazine;
2-[1-[(2,3-dihydro-1,4-benzodioxin-6-yl)methyl]-3-piperidinyl]-6-methyl-pyrazine;
2-[1-[(2,3-dihydro-1,4-benzodioxin-6-yl)methyl]-3-pyrrolidinyl]-4,6-dimethyl-pyrimidine;
2-[1-[(2,3-dihydro-1,4-benzodioxin-6-yl)methyl]-3-pyrrolidinyl]-4-methyl-pyrimidine;
2-[1-(1,3-benzodioxol-5-ylmethyl)-3-piperidinyl]-pyrazine;
6-[[3-(4,6-dimethyl-2-pyrimidinyl)-1-pyrrolidinyl]methyl]-quinoline;
2-[[[1-[(2,3-dihydro-1,4-benzodioxin-6-yl)methyl]-3-piperidinyl]oxy]methyl]-pyridine;
1-methyl-2-[[3-(4-pyrimidinyl)-1-piperidinyl]methyl]-1H-benzimidazole;
1-methyl-2-[[3-(4-methyl-2-pyrimidinyl)-1-pyrrolidinyl]methyl]-1H-benzimidazole;
1-ethyl-2-[[3-(4-pyridinyloxy)-1-pyrrolidinyl]methyl]-1H-benzimidazole;
1-methyl-2-[[3-(2-pyrazinyl)-1-piperidinyl]methyl]-1H-benzimidazole;
1-methyl-2-[[3-(6-methyl-2-pyrazinyl)-1-piperidinyl]methyl]-1H-benzimidazole;
2-[[3-(4-pyrimidinyl)-1-piperidinyl]methyl]-1H-benzimidazole;
2-[[3-(4,6-dimethyl-2-pyrimidinyl)-1-pyrrolidinyl]methyl]-1-methyl-1H-benzimidazole;
1-methyl-2-[[3-(3-pyridinylmethoxy)-1-piperidinyl]methyl]-1H-benzimidazole;
2-[3-(2-pyrazinyl)-1-piperidinyl]-1-(1-pyrrolidinyl)-ethanone;
2-[3-(3-pyridinylmethyl)-1-piperidinyl]-1-(1-pyrrolidinyl)-ethanone;
2-[3-(4-methylpyrimidin-2-yl)pyrrolidin-1-yl]-1-pyrrolidin-1-yl-ethanone; or
5-[[3-(3-pyridinylmethoxy)-1-piperidinyl]methyl]-2,1,3-benzothiadiazole;

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I′)

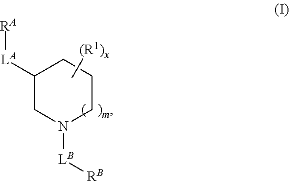

and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and

C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;

L^{A} is selected from the group consisting of a covalent bond, >O, >CH₂, —OCH₂—, —CH₂O—, >NH, and >NCH₃;

m represents 0 or 1;

x represents 0, 1 or 2;

each R¹, when present, is bound to any available carbon atom and is independently selected from the group consisting of halo and C₁₋₄alkyl optionally substituted with 1, 2, or 3 independently selected halo substituents; or two R¹ substituents are bound to the same carbon atom and form together a cyclopropylidene radical;

L^{B} is selected from the group consisting of >CHR² and >SO₂;

wherein R² is selected from the group consisting of hydrogen, and C₁₋₄alkyl optionally substituted with 1, 2 or 3 independently selected halo substituents; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

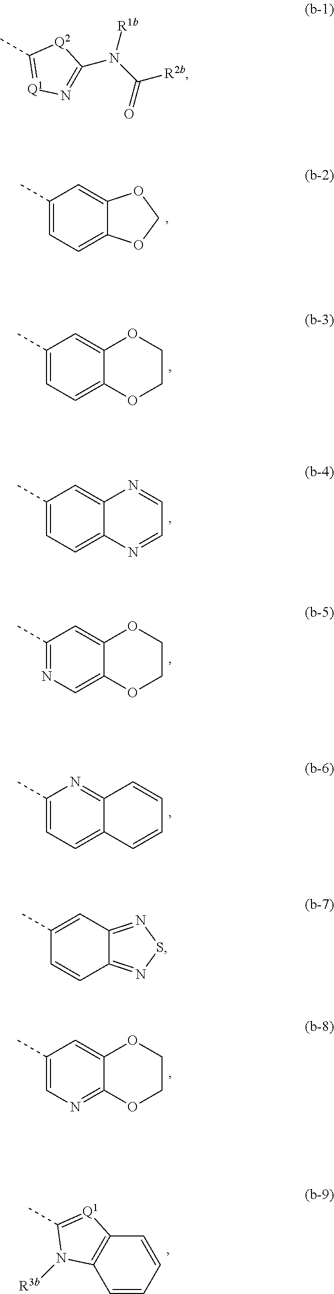

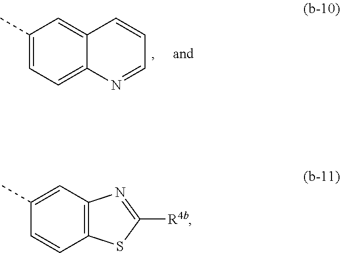

each Q¹ is CH or N;

Q² is O, NR^{q} or S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

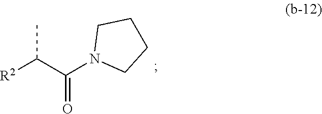

and the pharmaceutically acceptable salts and the solvates thereof,

for use as a medicament, in particular for use in preventing or treating a disorder mediated by the inhibition of O-GlcNAc hydrolase (OGA), and more in particular, in preventing or treating a tauopathy, such as Alzheimer's disease.

The present invention is also directed to compounds of Formula (I)

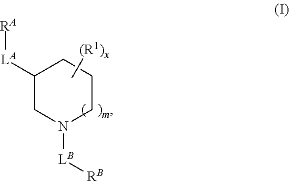

and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;

L^{A} is selected from the group consisting of a covalent bond, >O, >CH₂, —OCH₂—, —CH₂O—, >NH, and >NCH₃;

m represents 0 or 1;

x represents 0, 1 or 2;

each R¹, when present, is bound to any available carbon atom and is independently selected from the group consisting of halo and C₁₋₄alkyl optionally substituted with 1, 2, or 3 independently selected halo substituents; or two R¹ substituents are bound to the same carbon atom and form together a cyclopropylidene radical;

L^{B} is selected from the group consisting of >CHR² and >SO₂;

wherein R² is selected from the group consisting of hydrogen, and C₁₋₄alkyl optionally substituted with 1, 2 or 3 independently selected halo substituents; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

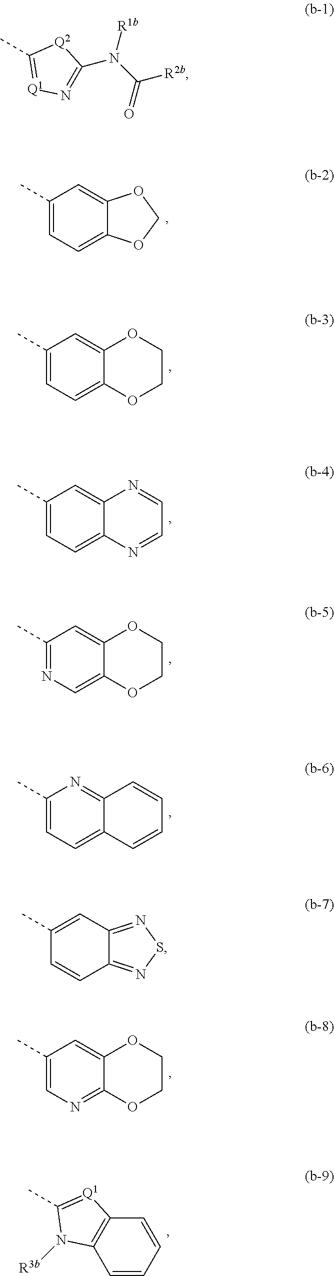

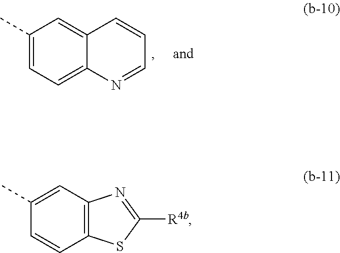

wherein

each Q¹ is CH or N;

Q² is O, NR^{q} or S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

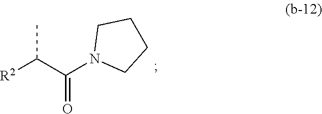

with the proviso that the compound is not
2-[1-[(2,3-dihydro-1,4-benzodioxin-6-yl)methyl]-3-piperidinyl]-pyrazine;
2-[1-[(2,3-dihydro-1,4-benzodioxin-6-yl)methyl]-3-piperidinyl]-6-methyl-pyrazine;
2-[1-[(2,3-dihydro-1,4-benzodioxin-6-yl)methyl]-3-pyrrolidinyl]-4,6-dimethyl-pyrimidine;
2-[1-[(2,3-dihydro-1,4-benzodioxin-6-yl)methyl]-3-pyrrolidinyl]-4-methyl-pyrimidine;
2-[1-(1,3-benzodioxol-5-ylmethyl)-3-piperidinyl]-pyrazine;
6-[[3-(4,6-dimethyl-2-pyrimidinyl)-1-pyrrolidinyl]methyl]-quinoline;
2-[[[1-[(2,3-dihydro-1,4-benzodioxin-6-yl)methyl]-3-piperidinyl]oxy]methyl]-pyridine;
1-methyl-2-[[3-(4-pyrimidinyl)-1-piperidinyl]methyl]-1H-benzimidazole;
1-methyl-2-[[3-(4-methyl-2-pyrimidinyl)-1-pyrrolidinyl]methyl]-1H-benzimidazole;
1-ethyl-2-[[3-(4-pyridinyloxy)-1-pyrrolidinyl]methyl]-1H-benzimidazole;
1-methyl-2-[[3-(2-pyrazinyl)-1-piperidinyl]methyl]-1H-benzimidazole;
1-methyl-2-[[3-(6-methyl-2-pyrazinyl)-1-piperidinyl]methyl]-1H-benzimidazole;
2-[[3-(4-pyrimidinyl)-1-piperidinyl]methyl]-1H-benzimidazole;
2-[[3-(4,6-dimethyl-2-pyrimidinyl)-1-pyrrolidinyl]methyl]-1-methyl-1H-benzimidazole;
1-methyl-2-[[3-(3-pyridinylmethoxy)-1-piperidinyl]methyl]-1H-benzimidazole;
2-[3-(2-pyrazinyl)-1-piperidinyl]-1-(1-pyrrolidinyl)-ethanone;
2-[3-(3-pyridinylmethyl)-1-piperidinyl]-1-(1-pyrrolidinyl)-ethanone;
2-[3-(4-methylpyrimidin-2-yl)pyrrolidin-1-yl]-1-pyrrolidin-1-yl-ethanone; or
5-[[3-(3-pyridinylmethoxy)-1-piperidinyl]methyl]-2,1,3-benzothiadiazole;

and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), or compounds of Formula (I′) for use, as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I′) as defined hereinbefore, and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; NR^{a}R^{aa}, wherein R^{a} and R^{aa} are each independently selected from the group consisting of hydrogen and C₁₋₄alkyl optionally substituted with 1, 2, or 3 independently selected halo substituents; and C₁₋₄alkyloxy optionally substituted with 1, 2, or 3 independently selected halo substituents;

L^{A} is selected from the group consisting of a covalent bond, >O, >CH₂, —OCH₂—, —CH₂O—, >NH, and >NCH₃;

m represents 0 or 1;

x represents 0, 1 or 2;

each R¹, when present, is bound to any available carbon atom and is independently selected from the group consisting of halo and C₁₋₄alkyl optionally substituted with 1, 2, or 3 independently selected halo substituents; or two R¹ substituents are bound to the same carbon atom and form together a cyclopropylidene radical;

L^{B} is selected from the group consisting of >CHR² and >SO₂;

wherein R² is selected from the group consisting of hydrogen, and C₁₋₄alkyl optionally substituted with 1, 2 or 3 independently selected halo substituents; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

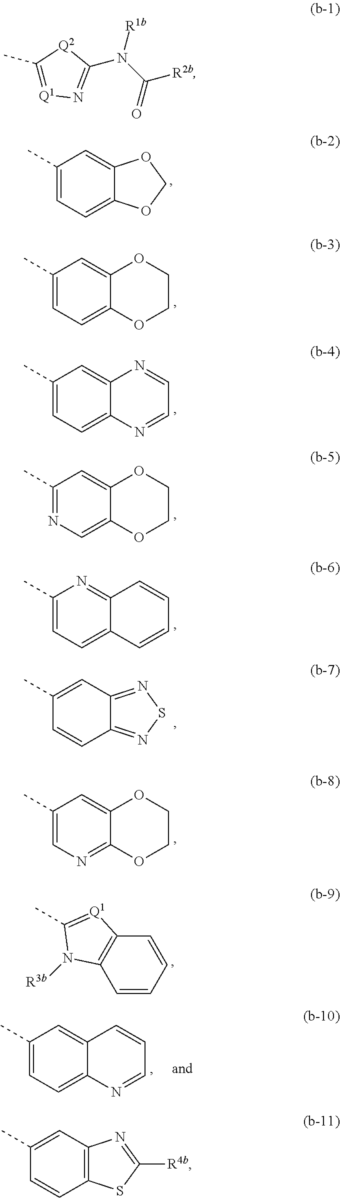

wherein

each Q¹ is CH or N;

Q² is O, NR^{q} or S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

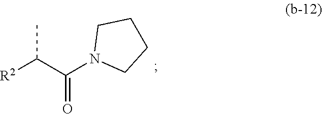

and the pharmaceutically acceptable salts and the solvates thereof,

for use as a medicament, in particular for use in preventing or treating a disorder mediated by the inhibition of O-GlcNAc hydrolase (OGA), and more in particular, in preventing or treating a tauopathy such as Alzheimer's disease.

In a particular embodiment, the invention is directed to compounds of Formula (I) as referred to herein, and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; NR^{a}R^{aa}, wherein R^{a} and R^{aa} are each independently selected from the group consisting of hydrogen and C₁₋₄alkyl optionally substituted with 1, 2, or 3 independently selected halo substituents; and C₁₋₄alkyloxy optionally substituted with 1, 2, or 3 independently selected halo substituents;

L^{A} is selected from the group consisting of a covalent bond, >O, >CH₂, —OCH₂—, —CH₂O—, >NH, and >NCH₃;

m represents 0 or 1;

x represents 0, 1 or 2;

each R¹, when present, is bound to any available carbon atom and is independently selected from the group consisting of halo and C₁₋₄alkyl optionally substituted with 1, 2, or 3 independently selected halo substituents; or two R¹ substituents are bound to the same carbon atom and form together a cyclopropylidene radical;

L^{B} is selected from the group consisting of >CHR² and >SO₂;

wherein R² is selected from the group consisting of hydrogen, and C₁₋₄alkyl optionally substituted with 1, 2 or 3 independently selected halo substituents; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

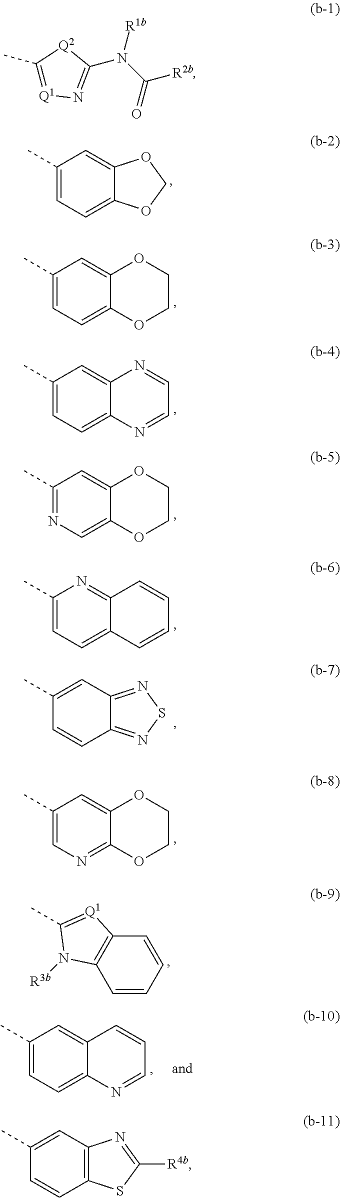

wherein

each Q¹ is CH or N;

Q² is O, NR^{q} or S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

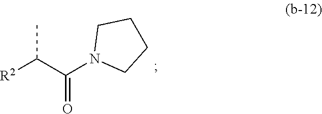

and the pharmaceutically acceptable salts and the solvates thereof.

In a particular embodiment, the invention is directed to compounds of Formula (I), or compounds of Formula (I′) for use, as referred to herein, and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; and C₁₋₄alkyloxy optionally substituted with 1, 2, or 3 independently selected halo substituents;

L^{A} is selected from the group consisting of a covalent bond, >O, >CH₂, —OCH₂—, —CH₂O—, >NH, and >NCH₃;

m represents 0 or 1;

x represents 0, 1 or 2;

each R¹, when present, is bound to any available carbon atom and is independently selected from the group consisting of halo and C₁₋₄alkyl optionally substituted with 1, 2, or 3 independently selected halo substituents;

L^{B} is selected from the group consisting of >CHR² and >SO₂;

wherein R² is selected from the group consisting of hydrogen, and C₁₋₄alkyl; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

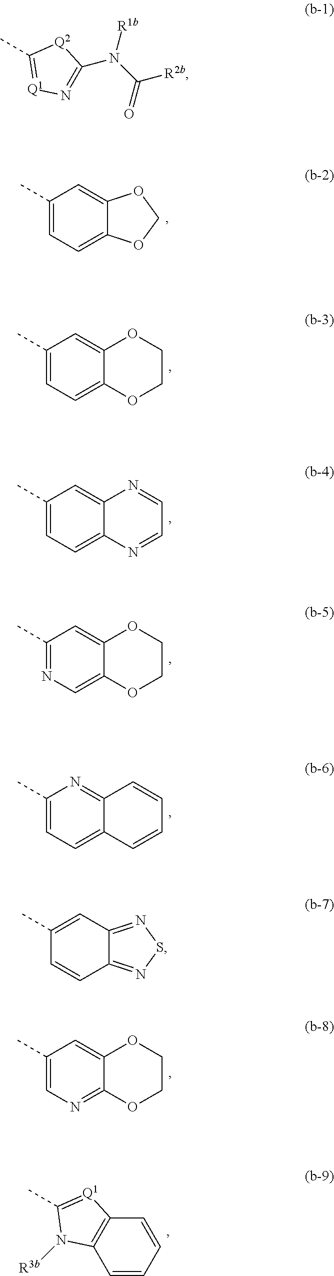

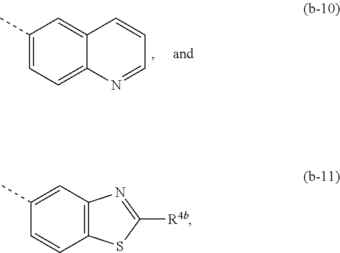

wherein

each Q¹ is CH or N;

Q² is O, NR^{q} or S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

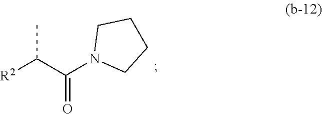

and the pharmaceutically acceptable salts and the solvates thereof.

In an additional embodiment, R^{A} is selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of fluoro; cyano; C₁₋₄alkyl optionally substituted with 1, 2, or 3 independently selected fluoro substituents; and C₁₋₄alkyloxy optionally substituted with 1, 2, or 3 independently selected fluoro substituents. More in particular, R^{A} as defined herein is optionally substituted 1 or 2 substituents each independently selected from the group consisting of fluoro; cyano; C₁₋₄alkyl, such as methyl, ethyl, isopropyl; CHF₂; CF₃; methoxy; ethoxy; and OCF₃.

In a further embodiment, the invention is directed to compounds of Formula (I), or compounds of Formula (I′) for use, as referred to herein, and the tautomers and the stereoisomeric forms thereof, wherein

R^{A} is a heteroaryl radical selected from the group consisting of pyridin-3-yl, pyridin-4-yl, and pyrimidin-4-yl, each of which may be optionally substituted with 1, 2 or 3 substituents each independently selected from C₁₋₄alkyl;

L^{A} is selected from the group consisting of a covalent bond, >O, >CH₂, —OCH₂—, —CH₂O—, >NH, and >NCH₃;

m represents 0 or 1;

x represents 0 or 1;

each R¹, when present, is bound to any available carbon atom and is independently selected from C₁₋₄alkyl;

L^{B} is selected from the group consisting of >CHR² and >SO₂;

wherein R² is hydrogen or C₁₋₄alkyl; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

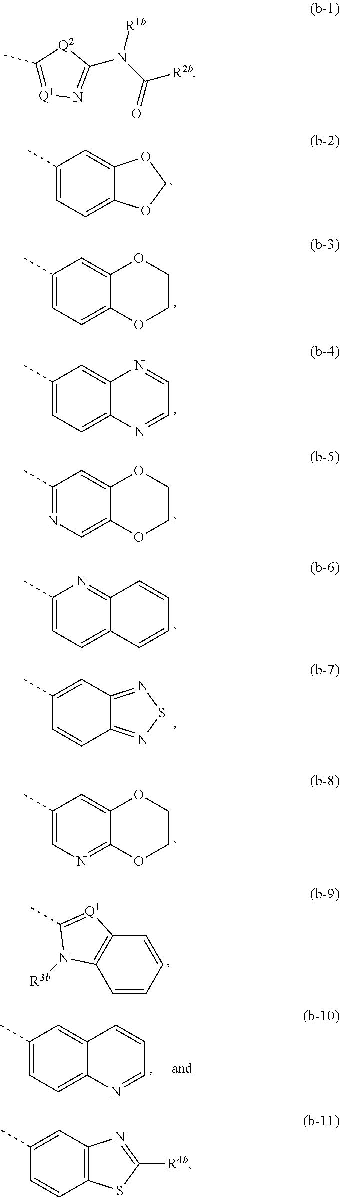

wherein

each Q¹ is CH or N;

Q² is O, NR^{q} or S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

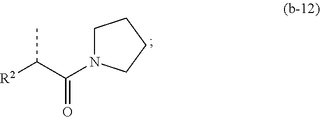

and the pharmaceutically acceptable salts and the solvates thereof.

In another embodiment, R^{B} is (b-1). In yet another embodiment, R^{B} is (b-2), (b-3), (b-4), (b-5), (b-6), (b-7), (b-8), (b-9), (b-10), or (b-11).

In a further embodiment, the invention is directed to compounds of Formula (I), or compounds of Formula (I′) for use, as referred to herein, and the tautomers and the stereoisomeric forms thereof, wherein

R^{A} is a heteroaryl radical selected from the group consisting of pyridin-3-yl, pyridin-4-yl, and pyrimidin-4-yl, each of which may be optionally substituted with 1, 2 or 3 substituents each independently selected from C₁₋₄alkyl;

L^{A} is selected from the group consisting of a covalent bond, >O, >CH₂, —OCH₂—, —CH₂O—, >NH, and >NCH₃;

m represents 0 or 1;

x represents 0;

L^{B} is selected from the group consisting of >CHR² and >SO₂;

wherein R² is hydrogen or C₁₋₄alkyl; and

R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²:

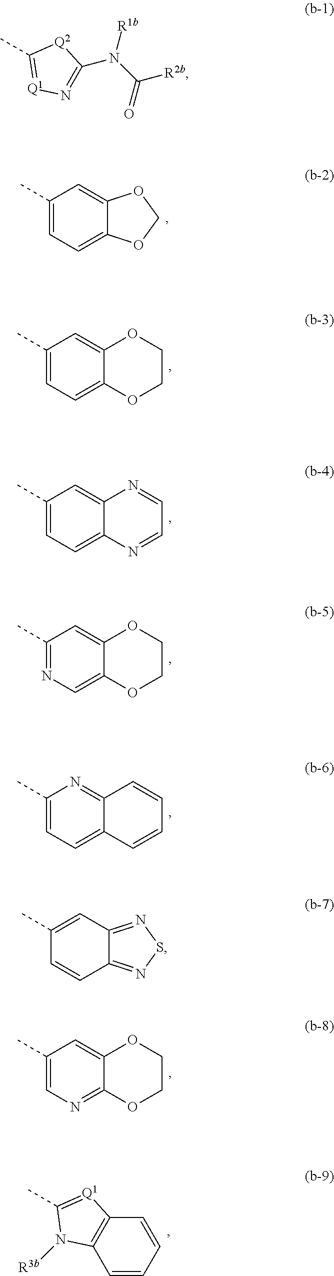

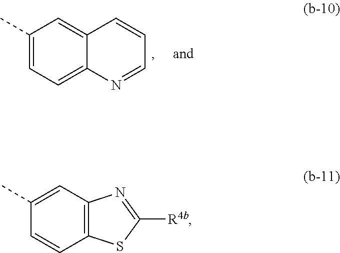

wherein

each Q¹ is CH;

Q² is S;

R^{1b} is H or C₁₋₄alkyl;

R^{2b} is C₁₋₄alkyl;

R^{3b}, R^{4b}, and R^{q} are each H or C₁₋₄alkyl;

or -L^{B}-R^{B} is (b-12)

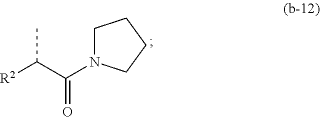

and the pharmaceutically acceptable salts and the solvates thereof.

In another embodiment, R^{B} is (b-1) or R^{B} is a radical selected from the group consisting of (b-2), (b-3), (b-4), (b-5), (b-6), (b-7), (b-8), (b-9), (b-10), and (b-11).

In another embodiment, R^{B} is (b-1), (b-2), (b-3), (b-4), (b-9) or (b-11). In yet another embodiment, R^{B} is (b-2), (b-3), (b-4), (b-9) or (b-11). In a further embodiment, R^{B} is (b-2), (b-3), (b-4), (b-9) and (b-11), wherein R^{3b} and R^{4b} are each hydrogen or methyl.

In a further embodiment, the invention is directed to compounds of Formula (I), or compounds of Formula (I′) for use, as referred to herein, and the tautomers and the stereoisomeric forms thereof, wherein

R^{A} is a heteroaryl radical selected from the group consisting of pyridin-3-yl, pyridin-4-yl, and pyrimidin-4-yl, each of which may be optionally substituted with 1, 2 or 3 substituents each independently selected from C₁₋₄alkyl;

L^{A} is selected from the group consisting of a covalent bond, >O, >CH₂, —OCH₂—, —CH₂O—, >NH, and >NCH₃;

m represents 0 or 1;

x represents 0;

L^{B} is selected from the group consisting of >CHR² and >SO₂;

wherein R² is hydrogen or C₁₋₄alkyl; and

R^{B} is (b-1) when L^{B} is >SO₂, or R^{B} is a radical selected from the group consisting of (b-1), (b-2), (b-3), and (b-4) when L^{B} is >CHR²:

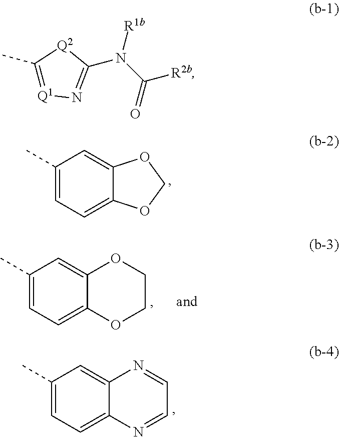

wherein

each Q¹ is CH;

Q² is S;

R^{1b} is H or C₁₋₄alkyl;

R^{2b} is C₁₋₄alkyl;

and the pharmaceutically acceptable salts and the solvates thereof.

In an embodiment, the compounds of Formula (I), or compounds of Formula (I′) for use, as described herein are in particular compounds of Formula (I-A),

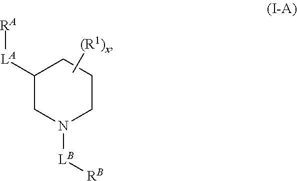

wherein all variables are as described in Formula (I) or (I′) herein.

In another embodiment, the compounds of Formula (I), or compounds of Formula (I′) for use, as described herein are in particular compounds of Formula (I-B),

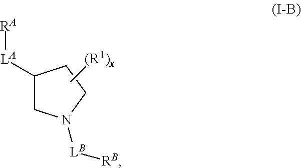

wherein all variables are as described in Formula (I) or (I′) herein.

In an additional embodiment, R^{A} is selected from the group consisting of

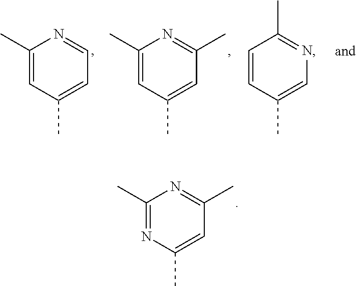

In an further embodiment, L^{A} is a covalent bond.

In an additional embodiment, L^{A} is selected from the group consisting of >O, >CH₂, —OCH₂—, —CH₂O—, >NH, and >NCH₃; in particular, L^{A} is >CH₂, —OCH₂—, or —CH₂O—; more in particular, L^{A} is >CH₂.

In another embodiment, L^{B} is —CH₂— or —CH(CH₃)—.

In a further embodiment, R^{B} is a radical selected from the group consisting of (b-1), (b-2), (b-4), in particular (b-1) and (b-4).

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before.

When L^{A} is defined, for the avoidance of doubt, it is defined from R^{A} to the pyrrolidine or piperidine ring. Thus, when L^{A} is defined as OCH₂, the O is bound to R^{A} and the CH₂ is bound to the pyrrolidine or piperidine ring.

The term “subject” as used herein, refers to an animal, preferably a mammal, most preferably a human, who is or has been the object of treatment, observation or experiment.

As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (+)-DL-lactic acid, lactobionic acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid,

p-toluenesulfonic acid, trifluoromethylsulfonic acid, and undecylenic acid. Representative bases which may be used in the preparation of pharmaceutically acceptable addition salts include, but are not limited to, the following: ammonia,

L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds according to Formula (I-a), can be prepared by reacting an intermediate compound of Formula (II) with a compound of Formula (XIV) according to reaction scheme (1). The reaction is performed in a suitable reaction-inert solvent, such as, for example, dichloromethane, in the presence of a suitable base, such as, for example, triethylamine, under thermal conditions 0° C. or room temperature, for example for 1 hour. In reaction scheme (1) all variables are defined as in Formula (I).

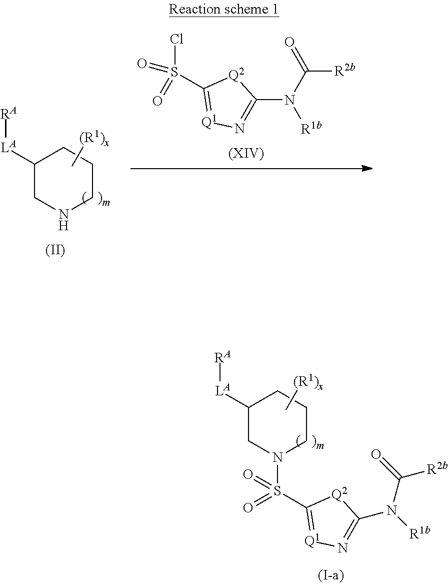

### Experimental Procedure 2

Additionally, final compounds of Formula (I-b) can be prepared by reacting an intermediate compound of Formula (II) with a compound of Formula (XV) according to reaction scheme (2). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (2) all variables are defined as in Formula (I).

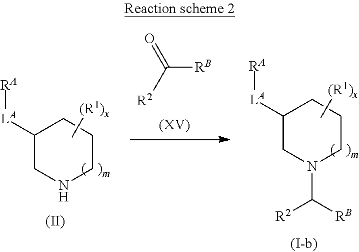

### Experimental Procedure 3

Additionally, final compounds of Formula (I-b) can be prepared by reacting an intermediate compound of Formula (II) with a compound of Formula (XVI) according to reaction scheme (3). The reaction is performed in a suitable reaction-inert solvent, such as, for example, acetonitrile, a suitable base, such as, for example, triethylamine or diisopropylethylamine, under thermal conditions, such as, 0° C. or room temperature, or 75° C., for example for 1 hour or 24 hours. In reaction scheme (3) all variables are defined as in Formula (I), and wherein halo is chloro, bromo or iodo.

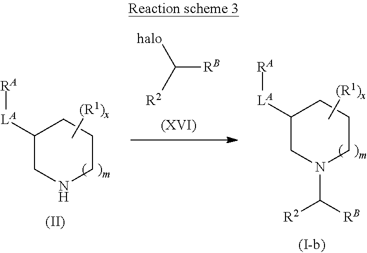

### Experimental Procedure 4

Additionally, final compounds of Formula (I-c) can be prepared by reacting an intermediate compound of Formula (II-a) with a compound of Formula (XVII) followed by reaction of the formed imine derivative with and intermediate compound of Formula (XVIII) according to reaction scheme (6). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. or room temperature, for example for 1 hour or 24 hours. In reaction scheme (6) all variables are defined as in Formula (I), and wherein R² is C₁₋₄alkyl, and halo is chloro, bromo or iodo

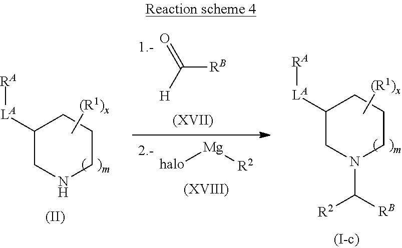

### Experimental Procedure 5

Intermediate compounds of Formula (II) can be prepared cleaving a protecting group in an intermediate compound of Formula (III) according to reaction scheme (5). In reaction scheme (5) all variables are defined as in Formula (I), and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known to the person skilled in the art and comprise but are not limited to: Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

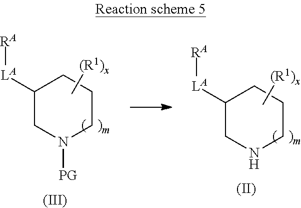

### Experimental Procedure 6

Intermediate compounds of Formula (III-a) can be prepared by “Nesighi coupling” reaction of a halo compound of Formula (IV) with an organozinc compound of Formula (V) according to reaction scheme (6). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (6) all variables are defined as in Formula (I), L^{A} is a bond or CH₂ and halo is preferably bromo or iodo. PG is defined as in Formula (III).

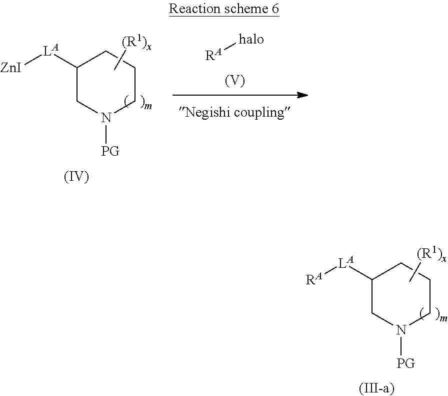

### Experimental Procedure 7

Intermediate compounds of Formula (IV) can be prepared by reaction of a halo compound of Formula (VI) with zinc according to reaction scheme (7). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (7) all variables are defined as in Formula (I), L^{A} is a bond or CH₂ and halo is preferably iodo. PG is defined as in Formula (III).

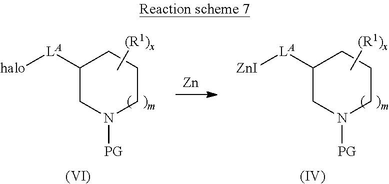

### Experimental Procedure 8

Intermediate compounds of Formula (III-b) can be prepared by hydrogenation reaction of an alkene compound of Formula (VII) according to reaction scheme (8). The reaction is performed in a suitable reaction-inert solvent, such as, for example, methanol, and a suitable catalyst, such as, for example, palladium on carbon, and hydrogen, under thermal conditions, such as, for example, room temperature, for example for 3 hours. In reaction scheme (8) all variables are defined as in Formula (I) and PG is defined as in Formula (III).

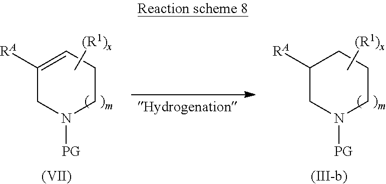

### Experimental Procedure 9

Intermediate compounds of Formula (VII) can be prepared by “Suzuki coupling” reaction of an alkene compound of Formula (VIII) and a halo derivative of Formula (V) according to reaction scheme (9). The reaction is performed in a suitable reaction-inert solvent, such as, for example, 1,4-dioxane, and a suitable catalyst, such as, for example, tetrakis(triphenylphosphine)palladium(0), a suitable base, such as, for example, NaHCO₃ (aq. sat. soltn.), under thermal conditions, such as, for example, 130° C., for example for 30 min under microwave irradiation. In reaction scheme (9) all variables are defined as in Formula (I), halo is preferably bromo or iodo, L^{A} is a bond, and PG is defined as in Formula (III).

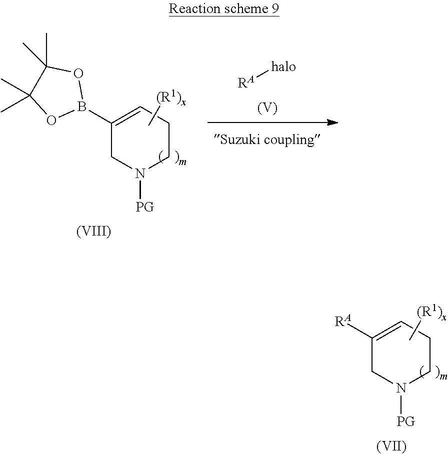

### Experimental Procedure 10

Intermediate compounds of Formula (III-c) can be prepared by reaction of a hydroxy compound of Formula (IX) and a halo derivative of Formula (V) according to reaction scheme scheme (10). The reaction is performed in a suitable reaction-inert solvent, such as, for example, dimethylformamide or dimethylsulfoxide, and a suitable base, such as, sodium hydride or potassium tert-butoxide, under thermal conditions, such as, for example, 50° C., for example for 48 hour. In reaction scheme (10) all variables are defined as in Formula (I), L^{A′} is a bond or CH₂ and halo is preferably chloro, bromo or fluoro. PG is defined as in Formula (III).

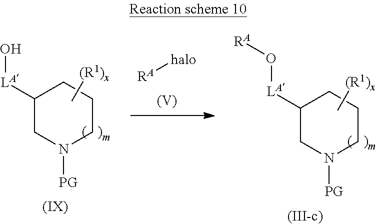

### Experimental Procedure 11

Alternatively, intermediate compounds of Formula (III-c) can be prepared by “Mitsunobu reaction” of a hydroxy compound of Formula (IX) and a hydroxy derivative of Formula (X) according to reaction scheme scheme (11). The reaction is performed in a suitable reaction-inert solvent, such as, for example, toluene, a phosphine, such as, triphenylphosphine, a suitable coupling agent, such as, for example DIAD (CAS: 2446-83-5), under thermal conditions, such as, for example, 70° C., for example for 17 hour. In reaction scheme (11) all variables are defined as in Formula (I), L^{A} is a bond or CH₂ and halo is preferably chloro, bromo or fluoro. PG is defined as in Formula (III).

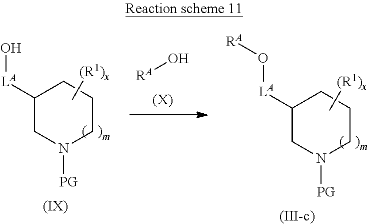

### Experimental Procedure 12

Intermediate compounds of Formula (III-d) can be prepared by “Buchwald coupling” reaction of an amino compound of Formula (XI) and a halo derivative of Formula (V) according to reaction scheme (12). The reaction is performed in a suitable reaction-inert solvent, such as, for example, 1,4-dioxane, and a suitable base, such as, sodium tert-butoxide, a suitable transition metal catalyst, such as, for example, tris(dibenzylideneacetone)dipalladium(0) (CAS: 51364-51-3), and a suitable ligand for the transition metal, such as, for example, 2-dicyclohexylphosphino-2′-(N,N-dimethylamino)biphenyl (CAS: 213697-53-1), under thermal conditions, such as, for example, 100° C., for example for 16 hour. In reaction scheme (12) all variables are defined as in Formula (I), L^{A} is a bond and halo is preferably chloro or bromo. PG is defined as in Formula (III).

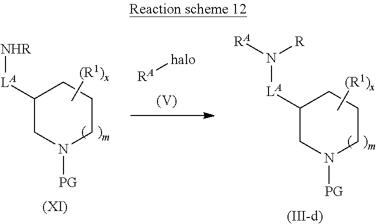

### Experimental Procedure 13

Intermediate compounds of Formula (III-e) can be prepared by alkylation reaction of an intermediate compound of Formula (XII) and a halo derivative of Formula (XIII) according to reaction scheme (13). The reaction is performed in a suitable reaction-inert solvent, such as, DMF, and a suitable base, such as, sodium hydride, under thermal conditions, such as, for example, room temperature, for example for 18 hour. In reaction scheme (12) all variables are defined as in Formula (I), L^{A′} is O, NH or NMe and halo is preferably chloro or bromo or iodo. PG is defined as in Formula (III).

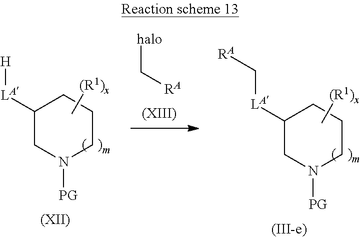

Intermediates of Formula, (V), (VI) (VIII), (IX) (XI), (XII), (XIII), (XIV), (XV), (XVI), (XVII) and (XVIII) are commercially available or can be prepared by know procedures to those skilled in the art.

Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I′) or (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I′) or (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and tauopathy diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I′) or (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I′) or (I) and one or more additional therapeutic agents, as well as administration of the compound of Formula (I′) or (I) and each additional therapeutic agent in its own separate pharmaceutical dosage formulation. For example, a compound of Formula (I′) or (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I′) or (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I′) or (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I′) or (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### EXPERIMENTAL PART

Hereinafter, the term “m.p.” means melting point, “min” means minutes, “ACN” means acetonitrile, “aq.” means aqueous, “Boc” means tert-butyloxycarbonyl, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “ⁱPrOH” means isopropyl alcohol, “RP” means reversed phase, “Rt” means retention time (in minutes), “[M+H]⁺” means the protonated mass of the free base of the compound, “wt” means weight, “THF” means tetrahydrofuran, “Et₂O” means diethylether, “EtOAc” means ethyl acetate, “DCM” means dichloromethane, “MeOH” means methanol, “sat” means saturated, “soltn” means solution, “sol.” means solution, “EtOH” means ethanol, “TFA” means trifluoroacetic acid, “2-meTHF” means 2-methyl-tetrahydrofuran, “NMP” means N-methylpyrrolidone, “Pd(OAc)₂” or “(OAc)₂Pd” means palladium(II) acetate, “Pd2(dba)₃” means tris(dibenzylideneacetone)dipalladium(0), “RuPhos” means 2-dicyclohexylphosphino-2′,6′-diisopropoxybiphenyl, and “TMSCl” means trimethylsilyl chloride.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “R*” or “S*” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Flow chemistry reactions were performed in a Vapourtec R2+R4 unit using standard reactors provided by the vendor.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 m (normal phase disposable flash columns) on different flash systems: either a SPOT or LAFLASH systems from Armen Instrument, or PuriFlash®430evo systems from Interchim, or 971-FP systems from Agilent, or Isolera 1SV systems from Biotage.

### A. PREPARATION OF THE INTERMEDIATES

### Preparation of Intermediates 1, 1a and 1b

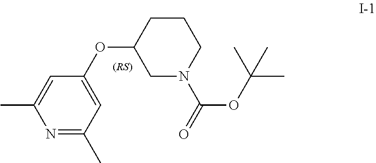 [SMILES: Cc1cc(OC2CCCN(C(=O)OC(C)(C)C)C2)cc(C)n1]

Sodium hydride (1 g, 25 mmol) was added to 1-Boc-3-hydroxypiperidine (CAS: 85275-45-2; 5 g, 25 mmol) in DMF (100 mL) at 0° C. The mixture was allowed to warm to rt and then it was cooled again to 0° C. A solution of 2,6-dimethyl-4-chloropyridine (CAS: 3512-75-2; 3.52 g, 25 mmol) in DMF (10 mL) was added dropwise. The mixture was stirred at 50° C. for 60 h. Then the mixture was cooled to rt. Water was added and the mixture was extracted with EtOAc. The organic layer was dried over MgSO₄, filtered and evaporated under vacuum. The resulting residue was purified by flash chromatography (silica gel, DCM, 1% MeOH in DCM, 2%, 4%) The pure fractions were evaporated under vacuum affording intermediate 1 (2.52 g, 33%).

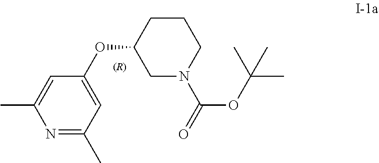

Intermediate 1a was prepared from (R)-1-Boc-3-hydroxypiperidine (CAS: 143900-44-1) following the procedure used for the preparation of intermediate 1.

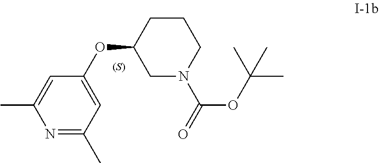

Intermediate 1b was prepared from (s)-1-Boc-3-hydroxypiperidine (CAS: 143900-43-0) following the procedure used for the preparation of intermediate 1.

### Preparation of Intermediate 2, 2a and 2b

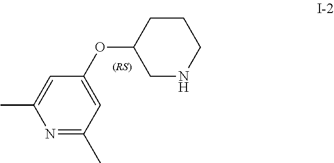

To a mixture of intermediate 1 (2.52 g, 8.2 mmol) in MeOH (50 mL) at rt, HCl (50 mL, 6M solution in i-PrOH) was added and the mixture was stirred at rt for 2 h. The volatiles were evaporated under vacuum. The resulting residue was taken up in acetonitrile and the formed crystals were filtered off and dried affording intermediate 2 as a bis HCl salt (1.52 g, 66%).

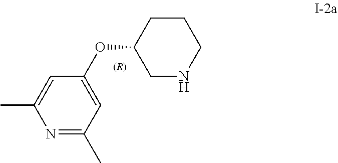

Intermediate 2a was prepared from intermediate 1a following the procedure used for the preparation of intermediate 2.

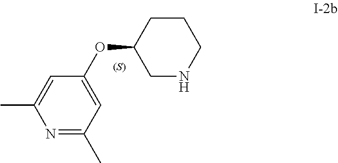

Intermediate 2b was prepared from intermediate 1b following the procedure used for the preparation of intermediate 2.

### Preparation of Intermediate 3

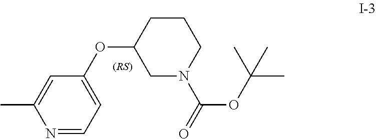 [SMILES: Cc1cc(OC2CCCN(C(=O)OC(C)(C)C)C2)ccn1]

Sodium hydride (1 g, 25 mmol) was added to 1-Boc-3-hydroxypiperidine (CAS: 85275-45-2; 5 g, 25 mmol) in DMF (100 mL) at 0° C. The mixture was allowed to warm to rt and then it was cooled again to 0° C. A solution of 2-methyl-4-chloropyridine (CAS: 3678-63-5; 3.17 g, 25 mmol) in DMF (10 mL) was added dropwise. The mixture was stirred at 60° C. for 16 h. Then the mixture was cooled to rt. The volatiles were evaporated in vacuo. Water was added and the mixture was extracted with EtOAc. The organic layer was dried over MgSO₄, filtered and evaporated under vacuum. affording intermediate 3 (7 g, 96%).

### Preparation of Intermediate 4

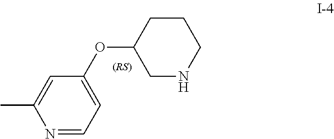

To a mixture of intermediate 3 (7 g, 24 mmol) in MeOH (100 mL) at rt, HCl (100 mL, 6M solution in i-PrOH) was added and the mixture was stirred at rt for 2 h. The volatiles were evaporated under vacuum. The resulting residue was taken up in i-PrOH and the formed crystals were filtered off and dried affording intermediate 4 as a bis HCl salt (3.78 g, 59%).

### Preparation of Intermediate 5

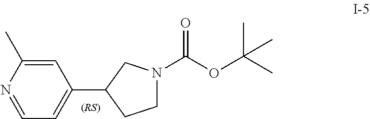 [SMILES: Cc1cc(C2CCN(C(=O)OC(C)(C)C)C2)ccn1]

A solution of tert-butyl 3-iodopyrrolidine-1-carboxylate (0.86 g, 2.9 mmol) in THF (6 mL) was pumped using the vapourtec R2+R4 through a column containing activated Zn (15 g, 229 mmol) at a flow of 0.5 mL/min at 40° C. The outcome solution was collected over a solution of 4-bromo-2-methylpyridine (0.17 mL, 1.45 mmol), Pd(OAc)₂ (16 mg, 0.073 mmol) and 2-dicyclohexylphosphino-2′,6′-di-iso-propoxy-1,1′-biphenyl (also known as RuPhos) (CAS: 787618-22-8; 11.68 mg, 0.14 mmol) in THF (1.5 mL) at rt. The mixture was stirred at rt for 16 h. 10% aqueous NH₄Cl was added and the mixture was extracted with EtOAc. The organic layer was separated and concentrated in vacuo. The residue thus obtained was purified by flash column chromatography (silica; EtOAc in DCM, 0/100 to 100/0, then MeOH in EtOAc, 0/100 to 20/80) and the desired fractions were concentrated in vacuo to yield intermediate 5 as yellow oil (155 mg, 41% yield).

### Preparation of Intermediate 6

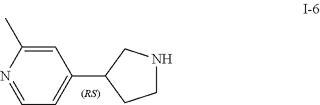

HCl (1.5 mL, 4M solution in 1,4-dioxane) was added to intermediate 5 (155 mg, 0.514 mmol) at it. The mixture was stirred at rt for 30 min. The volatiles were evaporated under vacuum affording intermediate 6 as a bis HCl salt as a yellow sticky solid (121 mg, quantitative).

### Preparation of Intermediate 7

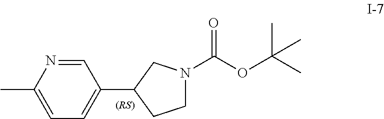 [SMILES: Cc1ccc(C2CCN(C(=O)OC(C)(C)C)C2)cn1]

A solution of tert-butyl 3-iodopyrrolidine-1-carboxylate (1.1 g, 3.7 mmol) in THF (7.4 mL) was pumped using the vapourtec R2+R4 through a column containing activated Zn (15 g, 229 mmol) at a flow of 0.5 mL/min at 40° C. The outcome solution was collected over a solution of 4-bromo-2-methylpyridine (0.17 mL, 1.45 mmol), Pd(OAc)₂ (16 mg, 0.073 mmol) and 2-dicyclohexylphosphino-2′,6′-di-iso-propoxy-1,1′-biphenyl (also known as RuPhos) (CAS: 787618-22-8; 11.68 mg, 0.14 mmol) in THF (1.6 mL) at rt and under N₂ atmosphere. The mixture was stirred at rt for 16 h. 10% aqueous NH₄Cl was added and the mixture was extracted with EtOAc. The organic layer was separated and concentrated in vacuo. The residue thus obtained was purified by flash column chromatography (silica; EtOAc in DCM, 0/100 to 100/0) and the desired fractions were concentrated in vacuo to yield intermediate 7 as yellow oil (302 mg, 85% pure, 67% yield).

### Preparation of Intermediate 8

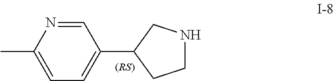

Trifluoroacetic acid (0.25 mL, 3.24 mmol) was added to a solution of intermediate 7 (100 mg, 85% pure, 0.324 mmol) at rt. The mixture was stirred at rt for 2 h. The volatiles were evaporated under vacuum affording intermediate 8 as a bis trifluoroacetate salt as a red oil (89 mg, quantitative).

### Preparation of Intermediate 9

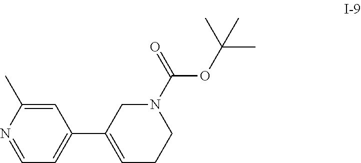 [SMILES: Cc1cc(C2=CCCN(C(=O)OC(C)(C)C)C2)ccn1]

To a mixture of 1-Boc-5,6-dihydro-2H-pyridine-3-boronic acid pinacol ester (CAS: 885693-20-9; 600 mg, 1.94 mmol) and NaHCO₃ (1.94 mL, 3.88 mmol, 2M solution in water) in 1,4-dioxane (20 mL), 4-bromo-2-methylpyridine (0.23 mL, 1.94 mmol) and tetrakis(triphenylphosphine)palladium(0) (112 mg, 0.097 mmol) were added at rt while N₂ was bubbled through the solution. The mixture was heated at 130° C. for 20 min in a sealed tube under microwave irradiation. Water and EtOAc were added and the organic layer was separated, dried over MgSO₄, filtered and evaporated under vacuum. The residue thus obtained was purified by flash column chromatography (silica; EtOAc in heptane, 1/3 to 4/1) and the desired fractions were concentrated in vacuo affording intermediate 9 (170 mg, 32% yield).

### Preparation of Intermediate 10

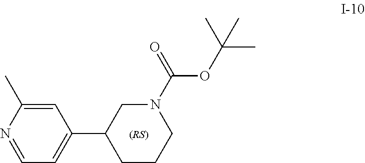 [SMILES: Cc1cc(C2CCCN(C(=O)OC(C)(C)C)C2)ccn1]

A mixture of intermediate 9 (170 mg, 0.62 mmol) in MeOH (14 mL) and palladium on carbon (19.78 mg; 0.19 mmol) was hydrogenated (atmospheric pressure) at rt for 3 h. The resulting mixture was filtered through a Celite® pad and the filtrate was evaporated in vacuo affording intermediate 10 (146 mg, 85% yield).

### Preparation of Intermediate 11

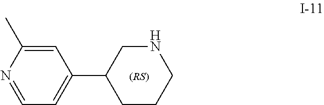

HCl (1.32 mL, 4M solution in 1,4-dioxane) was added to intermediate 10 (146 mg, 0.528 mmol) at rt. The mixture was stirred at rt for 2 h. The volatiles were evaporated under vacuum affording intermediate 11 as a bis HCl salt (quantitative).

### Preparation of Intermediate 12

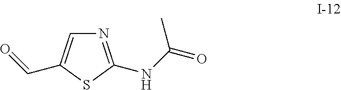

Acetyl choride (6 mL, 84.38 mmol) was added to a solution of 2-amino-5-formylthiazole (10 g, 78 mmol) and diisopropylamine (45 mL, 261.1 mmol) in DCM (100 mL) at 0° C. The resulting mixture was allowed to warm to rt and further stirred at rt for 17 h. NH₄Cl (aq. sat. soltn.) was added and the mixture was extracted with EtOAc. The organic layer was separated, dried over MgSO₄, filtered and concentrated in vacuo. The residue thus obtained was purified by flash column chromatography (silica; dry load, EtOAc in DCM 0/100 to 50/50) and the desired fractions were concentrated in vacuo to yield intermediate 12 as yellow solid (8.6 g, 65% yield).

### Preparation of Intermediate 13

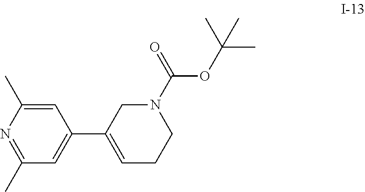 [SMILES: Cc1cc(C2=CCCN(C(=O)OC(C)(C)C)C2)cc(C)n1]

To a mixture of 1-Boc-5,6-dihydro-2H-pyridine-3-boronic acid pinacol ester (CAS: 885693-20-9; 700 mg, 2.26 mmol) and NaHCO₃ (2.26 mL, 4.53 mmol, 2M solution in water) in 1,4-dioxane (23.1 mL), 4-bromo-2,6-dimethylpyridine (430 mg, 2.26 mmol) and tetrakis(triphenylphosphine)palladium(0) (130 mg, 0.113 mmol) were added at rt while N₂ was bubbled through the solution. The mixture was heated at 130° C. for 20 min in a sealed tube under microwave irradiation. Water and EtOAc were added and the organic layer was separated, dried over MgSO₄, filtered and evaporated under vacuum. The residue thus obtained was purified by flash column chromatography (silica; EtOAc in heptane, 1/3 to 4/1) and the desired fractions were concentrated in vacuo affording intermediate 13 (213 mg, 33% yield).

### Preparation of Intermediate 14

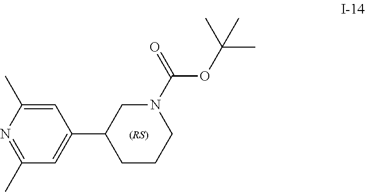 [SMILES: Cc1cc(C2CCCN(C(=O)OC(C)(C)C)C2)cc(C)n1]

A mixture of intermediate 13 (245 mg, 0.85 mmol) in MeOH (19 mL) and palladium on carbon (27.12 mg; 0.25 mmol) was hydrogenated (atmospheric pressure) at rt for 3 h. The resulting mixture was filtered through a Celite® pad and the filtrate was evaporated in vacuo affording intermediate 14 (239 mg, 97% yield).

### Preparation of Intermediate 15

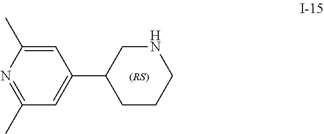

HCl (2.06 mL, 4M solution in 1,4-dioxane) was added to intermediate 14 (239 mg, 0.823 mmol) at rt. The mixture was stirred at rt for 4 h. The volatiles were evaporated under vacuum affording intermediate 15 as a bis HCl salt (quantitative).

### Preparation of Intermediate 16

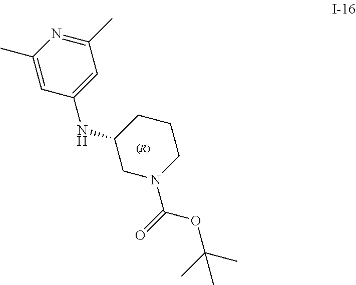

To a mixture of tris(dibenzylideneacetone)dipalladium(0) (CAS: 51364-51-3; 52 mg, 0.057 mmol), 2-dicyclohexylphosphino-2′-(N,N-dimethylamino)biphenyl (CAS: 213697-53-1; 41 mg, 0.104 mmol) and sodium tert-butoxide (154 mg, 1.6 mmol) in 1,4-dioxane (5 mL) at rt and under N₂ atmosphere, (R)-(−)-3-amino-1-Boc-piperidine (CAS: 188111-79-7; 0.23 mL, 1.2 mmol) and 4-chloro-2,6-dimethylpyridine (0.127 mL, 1 mmol) were added. The mixture was heated at 100° C. for 16 h in a sealed tube. Brine and DCM were added and the organic layer was separated, dried over MgSO₄, filtered and evaporated under vacuum. The residue thus obtained was purified by flash column chromatography (SiO₂ amino functionalized; EtOAc in heptane, 0/100 to 100/0) and the desired fractions were concentrated in vacuo affording intermediate 16 as a yellow oil (248 mg, 81% yield).

### Preparation of Intermediate 17

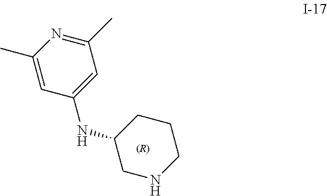

HCl (2 mL, 4M solution in 1,4-dioxane) was added to a solution of intermediate 16 (240 mg, 0.79 mmol) in 1,4-dioxane (4 mL) at rt and under N₂ atmosphere in a sealed tube. The mixture was stirred at rt for 16 h. The volatiles were evaporated under vacuum and the crude product was purified by ion exchange chromatography (Isolute® SCX-2, MeOH and then 7N solution of NH₃ in MeOH). The desired fractions were collected and concentrated in vacuo affording intermediate 17 as pale yellow oil (157 mg; 97% yield).

### Preparation of Intermediate 18

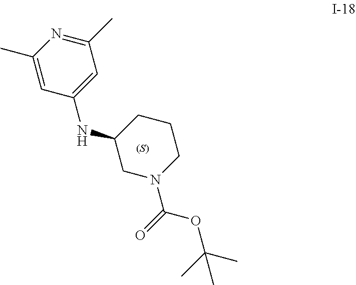

To a mixture of tris(dibenzylideneacetone)dipalladium(0) (CAS: 51364-51-3; 57 mg, 0.062 mmol), 2-dicyclohexylphosphino-2′-(N,N-dimethylamino)biphenyl (CAS: 213697-53-1; 33 mg, 0.084 mmol) and sodium tert-butoxide (135 mg, 1.40 mmol) in 1,4-dioxane (5 mL) at rt and under N₂ atmosphere, (S)-(−)-3-amino-1-Boc-piperidine (CAS: 216854-23-8; 0.23 mL, 1.2 mmol) and 4-chloro-2,6-dimethylpyridine (0.127 mL, 1 mmol) were added. The mixture was heated at 100° C. for 16 h in a sealed tube. Brine and DCM were added and the organic layer was separated, dried over MgSO₄, filtered and evaporated under vacuum. The residue thus obtained was purified by flash column chromatography (SiO₂ amino functionalized; EtOAc in heptane, 0/100 to 100/0) and the desired fractions were concentrated in vacuo affording intermediate 18 as a yellow oil (203 mg, 67% yield).

### Preparation of Intermediate 19

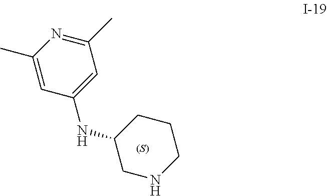

HCl (1.6 mL, 4M solution in 1,4-dioxane) was added to a solution of intermediate 18 (197 mg, 0.64 mmol) in 1,4-dioxane (3.5 mL) at rt and under N₂ atmosphere in a sealed tube. The mixture was stirred at rt for 16 h. The volatiles were evaporated under vacuum and the crude product was purified by ion exchange chromatography (Isolute® SCX-2, MeOH and then 7N solution of NH₃ in MeOH). The desired fractions were collected and concentrated in vacuo affording intermediate 19 as pale yellow oil (132 mg; 99% yield).

### Preparation of Intermediate 20

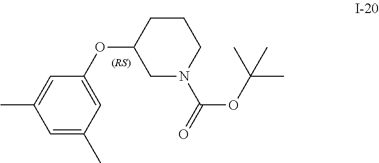 [SMILES: Cc1cc(C)cc(OC2CCCN(C(=O)OC(C)(C)C)C2)c1]

Diisopropyl azodicarboxylate (CAS: 2446-83-5; 1.2 mL, 6.17 mmol) was added to a mixture of triphenylphosphine (1.6 g, 6.1 mmol) in toluene (10 mL) at 0° C. Then a solution of 1-Boc-3-hydroxypiperidine (CAS: 85275-45-2; 1 g, 5 mmol) and 3,5-dimethylphenol (0.5 g, 4.1 mmol) in toluene (5 mL) was added and the mixture was stirred at 70° C. for 17 h. Water was added and the organic layer was separated, dried over MgSO₄, filtered and evaporated under vacuum affording crude intermediate 20 as a white solid (quantitative).

### Preparation of Intermediate 21

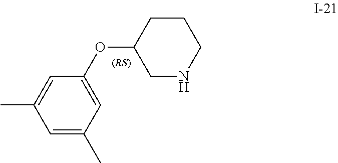

HCl (10 mL, 4M solution in 1,4-dioxane) was added to a solution of intermediate 20 (1.52 g, 4.96 mmol) in MeOH (10 mL) at rt. The mixture was stirred at rt for 2 h. The volatiles were evaporated under vacuum and the crude product was taken up in MeOH and amberlist 15—proton form (3.6 g, 14.76 mmol, loading 4.1 mmol/g) was added. The mixture was shaken at rt for 5 h. The resin was filtered off and washed with MeOH and the filtrates were discarded. The resin was suspended in a 7M solution of NH₃ in MeOH and was further shaken at rt for 2 h (twice). The resin was filtered off and washed with 7N solution of NH₃ in MeOH. The combined filtrates were concentrated in vacuo affording intermediate 21 as yellow oil (580 mg; 43% yield, 77% pure).

### Preparation of Intermediate 22

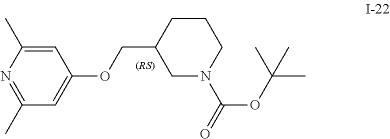 [SMILES: Cc1cc(OCC2CCCN(C(=O)OC(C)(C)C)C2)cc(C)n1]

Sodium hydride (67 mg, 1.67 mmol) was added to tert-butyl 3-(hydroxymethyl)piperidine-1-carboxylate (CAS: 116574-71-1; 300 mg, 1.4 mmol) in DMF (10 mL) at 0° C. The mixture was allowed to warm to rt and it was further stirred for 30 min. Then the mixture was cooled again to 0° C. and 4-bromo-2,6-dimethylpyridine (CAS: 5093-70-9; 285.2 mg, 1.53 mmol) was added. The mixture was stirred at rt overnight. Water was added and the mixture was extracted with EtOAc. The organic layer was dried over MgSO₄, filtered and evaporated under vacuum. The residue thus obtained was purified by flash column chromatography (SiO₂; EtOAc in heptane, 0/100 to 80/20) and the desired fractions were concentrated in vacuo affording intermediate 22 (65 mg, 16% yield).

Intermediate (3R)—I-22 was prepared following the same reaction procedure starting from tert-butyl 3R-(hydroxymethyl)piperidine-1-carboxylate and a stochiometric amount of 15-crown-5 ether.

### Preparation of Intermediate 23

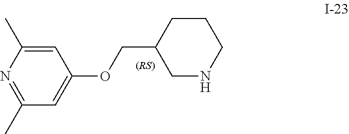 [SMILES: Cc1cc(OCC2CCCNC2)cc(C)n1]

HCl (0.57 mL, 4M solution in 1,4-dioxane) was added to intermediate 22 (65 mg, 0.203 mmol) at rt. The mixture was stirred at rt for 45 min. The volatiles were evaporated under vacuum affording intermediate 23 as a bis HCl salt (quantitative).

Intermediate (3R)—I-23 was prepared following the same reaction procedure starting from intermediate (3R)-22. m/z: [M+H]⁺ 221.2, Rt 0.43 min, method 13.

### Preparation of Intermediate 24

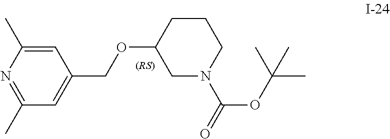 [SMILES: Cc1cc(COC2CCCN(C(=O)OC(C)(C)C)C2)cc(C)n1]

Sodium hydride (23.3 mg, 0.58 mmol) was added to 1-Boc-3-hydroxypiperidine (CAS: 85275-45-2; 111 mg, 0.55 mmol) in DMF (2.5 mL) at 0° C. and under N₂ atmosphere. The mixture was allowed to warm to rt and it was further stirred for 40 min. Then a solution of 4-bromomethyl-2,6-dimethylpyridine (CAS: 79313-02-3; 113 mg, 0.565 mmol) in DMF (2.5 mL) was added dropwise. The mixture was stirred at rt for 18 h. Water was added and the mixture was extracted with Et₂O. The organic layer was dried over MgSO₄, filtered and evaporated under vacuum. The residue thus obtained was purified by flash column chromatography (SiO₂; EtOAc in heptane, 0/100 to 100/0) and the desired fractions were concentrated in vacuo affording intermediate 24 as colourless oil (115 mg, 64% yield).

### Preparation of Intermediate 25

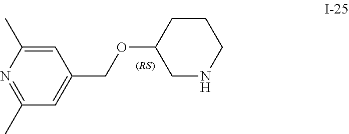 [SMILES: Cc1cc(COC2CCCNC2)cc(C)n1]

Trifluoroacetic acid (0.51 mL, 6.87 mmol) was added to a solution of intermediate 24 (110 mg, 0.34 mmol) in DCM (1.75 mL) at 0° C. The mixture was allowed to warm to rt and further stirred at rt for 2 h. The volatiles were evaporated under vacuum and the residue thus obtained was taken up in DCM and washed with K₂CO₃ (aq. sat. soltn.). The organic layer was dried over MgSO₄, filtered and evaporated under vacuum affording intermediate 25 (quantitative).

### Preparation of Intermediate 26

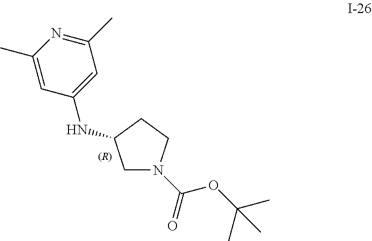

To a mixture of tris(dibenzylideneacetone)dipalladium(0) (CAS: 51364-51-3; 64 mg, 0.07 mmol), 2-dicyclohexylphosphino-2′-(N,N-dimethylamino)biphenyl (CAS: 213697-53-1; 38.6 mg, 0.098 mmol) and sodium tert-butoxide (202 mg, 2.1 mmol) in 1,4-dioxane (4 mL) at rt and under N₂ atmosphere, (R)-(−)-3-amino-1-Boc-pyrrolidine (CAS: 147081-49-0; 0.285 mL, 1.68 mmol) and 4-chloro-2,6-dimethylpyridine (0.178 mL, 1.4 mmol) were added. The mixture was heated at 100° C. for 18 h in a sealed tube. The reaction mixture was filtered over a pad of Dicalite® and rinsed with DCM. The filtrate was concentrated and the residue thus obtained was purified by flash column chromatography (SiO₂; 7N NH₃ in MeOH in DCM, 0/100 to 5/95) and the desired fractions were concentrated in vacuo affording intermediate 26 as a pale yellow solid (386 mg, 94% yield).

### Preparation of Intermediate 27

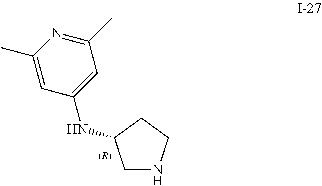

HCl (3.31 mL, 4M solution in 1,4-dioxane) was added to a solution of intermediate 26 (386 mg, 1.32 mmol) in 1,4-dioxane (3.33 mL) at rt. The mixture was stirred at rt for 1 h.

The volatiles were evaporated under vacuum affording a residue that was taken up in MeOH and passed through an Isolute® SCX-2 cartridge. The product was eluted with a 7N solution of NH₃ in MeOH. The volatiles were evaporated in vacuo affording intermediate 27 as colorless oil (93% yield).

### Preparation of Intermediate 28

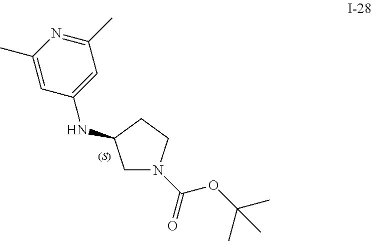

Intermediate 28 was prepared from (S)-(−)-3-amino-1-Boc-pyrrolidine (CAS 122536-76-9) following the same reaction procedure that the one for the preparation of intermediate 26.

### Preparation of Intermediate 29

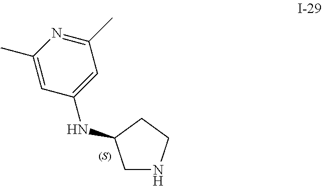

Intermediate 29 was prepared from intermediate 28 following the same reaction procedure as the one for the preparation of intermediate 27.

### Preparation of Intermediate 30

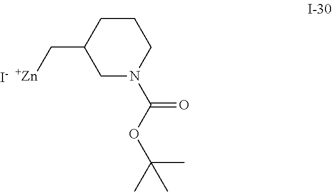

A solution of 3-iodomethylpiperidine-1-carboxylic acid tert-butyl ester (CAS: 253177-03-6; 1 g, 3.07 mmol) and LiCl (6.15 mL, 3.07 mmol, 0.5 M solution in THF) was pumped through a column containing activated Zn (12.3 g, 188.1 mmol) at 40° C. with flow of 0.5 mL/min. The outcome solution was collected under N₂ atmosphere to yield intermediate 30 as a clear solution that was used without any further manipulation.

For the above reaction Zn was activated as follows: A solution of TMSCl (2.2 mL) and 1-bromo-2-choroethane (0.5 mL) in THF (10 mL) was passed through the column containing Zn at a flow of 1 mL/min.

### Preparation of Intermediate (3S)-30

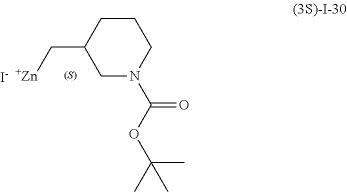

A solution of 3S-iodomethylpiperidine-1-carboxylic acid tert-butyl ester (CAS: 384829-99-6; 47.9 g, 147.3 mmol) in THF (292.8 mL) was pumped through a column containing activated zinc (14.45 g, 221 mmol) at 40° C. under N₂ at a flow rate of 1.5 mL/min. The resulting solution was collected over molecular sieves under N₂ atmosphere to yield intermediate (3S)-30 as a clear light brown solution. This solution was titrated twice against iodine in THF (0.34M) and used as such in the next step.

For the above reaction Zn was activated as follows: A solution of TMSCl (2.2 mL) and 1-bromo-2-choroethane (0.5 mL) in THF (10 mL) was passed through the column containing Zn at a flow of 1 mL/min.

### Preparation of Intermediate 31

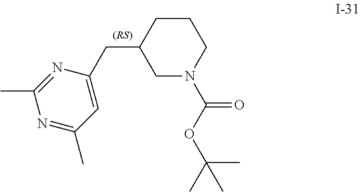

A solution of 4-chloro-2,6-dimethylpyrimidine (CAS: 4472-45-1; 731 mg, 5.13 mmol) in 0.5 M LiCl in THF (CAS: 109-99-9; 19.18 mL, 235.66 mmol) and intermediate 30 (7.69 mmol), was pumped using a Vapourtec R2+R4 through a column containing Siliacat DPP-Pd (4 g, 0.26 mmol/g, 1.04 mmol) at 80° C. and 0.1 mL/min (each). The column was washed with THF (20 mL). The outcome solution was quenched with water, extracted with EtOAc. The organic layer was separated, washed with brine, dried on MgSO₄ and evaporated. The residue thus obtained was purified on a column with silica gel, eluent: Heptane in EtOAc from 100% to 0%. The pure fractions were evaporated, yielding intermediate 31 (1.4 g, 89% yield) as a yellow sticky solid.

### Preparation of Intermediate 32

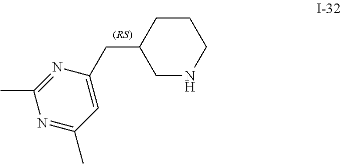

Trifluoroacetic acid (5.26 mL, 68.75 mmol) was added to a solution of intermediate 31 (1.4 g, 4.58 mmol) in DCM (7.7 mL) at rt. The mixture was further stirred at rt for 3 h. The volatiles were evaporated under vacuum and the residue thus obtained was taken up in DCM and washed with K₂CO₃ (aq. sat. soltn.). The organic layer was dried over MgSO₄, filtered and evaporated under vacuum affording crude intermediate 32 (quantitative).

### Preparation of Intermediate 33

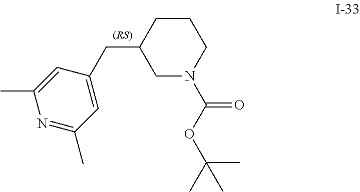

A solution of 4-bromo-2,6-dimethylpyrimidine (CAS: 5093-70-9; 762.5 mg, 4.09 mmol) in 0.5 M LiCl in THF (CAS: 109-99-9; 19.17 mL, 235.57 mmol) and intermediate 30 (6.15 mmol), was pumped using Vapourtec R2+R4 through a column containing Siliacat DPP-Pd (26.93 g, 0.26 mmol/g, 7 mmol) at 60° C. and 0.2 mL/min (each). The column was washed with THF (20 mL). The outcome, was quenched by the addition of water and extracted with EtOAc, the organic fraction was washed with brine, dried over MgSO₄ and evaporated. The residue was combined with 0.625 g from another batch which was obtained using the same procedure starting with 4-bromo-2,6-dimethylpyrimidine (CAS: 5093-70-9; 382.02 mg, 2.05 mmol). The residue was purified on a column with silica gel, eluent: heptane in EtOAc from 100% to 0%. The pure fractions were evaporated, yielding intermediate 33 (1.7 g, 90% yield) as a colorless oil.

### Preparation of Intermediate (3R)-33

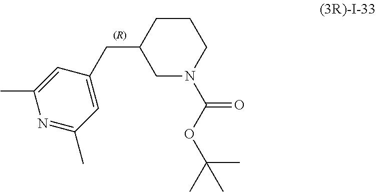

To a 400 mL reactor equipped with overhead stirrer and temperature probe, 4-bromo-2,6-dimethylpyridine (21 g, 113 mmol) was charged under N₂ atmosphere at rt. A THF solution of intermediate (3S)—I-30 (366 mL, 124.44 mmol, 0.34M solution in THF) was then added followed by N,N,N′,N′-tetramethylethylenediamine (18.66 mL, 124.4 mmol) and contents were degassed by N₂ sparging (5 min). Bis(triphenylphosphine)palladium(II) dichloride (CAS: 13965-03-2; 1.588 g, 2.263 mmol) was then added and contents degassed again by N₂ sparging for another 5 min. After this, the reaction mixture was warmed to 50° C. and stirred at this temperature for 1 h. The reaction mixture was then cooled down to 20° C. and quenched with a 1:1 mixture of 32% aq. NH₃ and sat. NH₄Cl (200 mL). Water (100 mL) was added followed by EtOAc (200 mL). The resulting biphasic solution was filtered through a pad of Celite® to remove the palladium black residue. Phases were then separated and aqueous back-extracted with EtOAc (200 mL). Combined organic extracts were dried over MgSO₄, solids filtered and solvents distilled under reduced pressure to dryness. Crude material was purified by normal phase column chromatography (silica, EtOAc in heptane 0/100 to 50/50). Desired fractions were collected and concentrated under reduced pressure to yield intermediate (3R)-33 (34.44 g, 89% yield) as an orange oil.

### Preparation of Intermediate 34

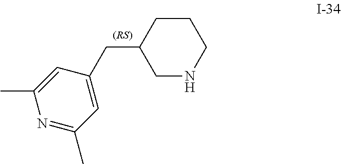

Trifluoroacetic acid (5.38 mL, 70.36 mmol) was added to a solution of intermediate 33 (1.7 g, 4.7 mmol) in DCM (7.9 mL) at rt. The mixture was further stirred at rt for 3 h. The volatiles were evaporated under vacuum and the residue thus obtained was taken up in DCM and washed with K₂CO₃ (aq. sat. soltn.). The organic layer was dried over MgSO₄, filtered and evaporated under vacuum affording crude intermediate 34 (quantitative).

### Preparation of Intermediate (3R)-34

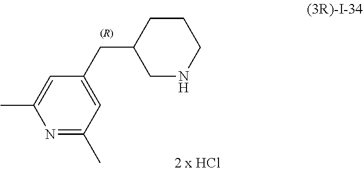

A 2-MeTHF (182.6 mL) solution of intermediate (3R)-33 (18.26 g, 59.98 mmol) was charged to a 400 mL reactor equipped with overhead stirrer under nitrogen. The resulting clear orange solution was cooled down to 0° C. and HCl (149.9 mL, 599.8 mmol, 4M solution in 1,4-dioxane) was added dropwise, maintaining the internal temperature below 5° C. Reaction mixture was stirred for 30 min at this temperature and warmed to 20° C. afterwards. A solid (bis HCl salt) crystallized with time. After 1 h at 20° C., the slurry was warmed to 50° C. and stirred for an extra 2 h. After that time, contents were cooled down to 0° C. and slurry filtered off. The wet cake was washed with 2-MeTHF (50 mL) and dried under vacuum at 50° C. overnight to yield intermediate (3R)-34 (16.18 g, 97% yield) as a white solid. m/z [M+H]⁺ 205.2, Rt 0.34 min, method 13; OR −4.1° (589 nm, c 0.53 w/v %, MeOH, 20° C.).

### Preparation of Intermediate 35

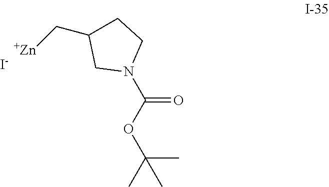

A solution of 3-iodomethylpyrrolidine-1-carboxylic acid tert-butyl ester (CAS: 479622-36-1; 0.93 g, 3 mmol) in THF (6 mL) was pumped through a column containing activated Zn (12 g, 183.5 mmol) at 40° C. with flow of 0.5 mL/min. The outcome solution was collected under N₂ atmosphere to yield intermediate 35 as a clear solution that was used without any further manipulation.

For the above reaction Zn was activated as follows: A solution of TMSCl (0.75 mL) and 1-bromo-2-choroethane (0.3 mL) in THF (10 mL) was passed through the column containing Zn at 40° C. with a flow of 1 mL/min.

### Preparation of Intermediate 36

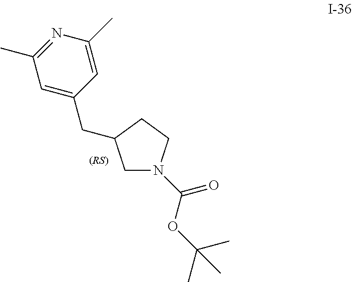 [SMILES: Cc1cc(CC2CCN(C(=O)OC(C)(C)C)C2)cc(C)n1]

A solution of 4-chloro-2,6-dimethylpyrimidine (CAS: 3512-75-2; 203.1 mg, 1.43 mmol) and intermediate 35 (7.17 mL, 0.3 M solution in THF) in THF (6.76 mL) was pumped using a Vapourtec R2+R4 through a column containing Siliacat DPP-Pd (9.22 g, 0.26 mmol/g, 2.4 mmol) at 80° C. and 0.2 mL/min (each). The column was washed with THF (20 mL). The outcome solution was quenched with water, extracted with EtOAc. The organic phase was separated dried over Na₂SO₄ and evaporated. The residue thus obtained was by automated flash chromatography (silica, EtOAc in heptane, from 0/100 to 80/20). The pure fractions were evaporated, yielding intermediate 36 (103 mg, 18% yield, 77% pure) as a dark orange oil.

### Preparation of Intermediate (3S)-36

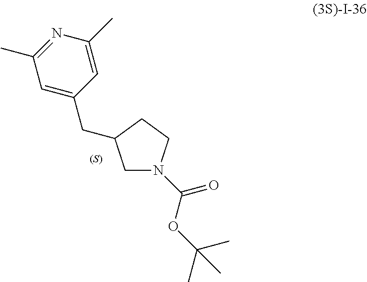

A solution of tert-butyl (3S)-3-(iodomethyl)pyrrolidine-1-carboxylate (CAS: 224168-68-7; 28.03 g, 90.8 mmol) in lithium chloride (165 mL, 0.5 M in THF) was pumped through a column containing activated zinc (11.66 g, 178.3 mml) at a flow of 0.4 mL/min at 40° C. The outlet solution was combined with a solution of 4-bromo-2,6-dimethylpyridine (10.05 g, 54.05 mmol) in lithium chloride (175 mL, 0.5 M in THF) at a flow of 0.4 mL/min. The combined streams were pumped through a column containing Siliacat DPP-Pd (1 g, 0.26 mmol/g, 0.26 mmol) at 60° C. and a flow of 0.4 mL/min (each). The column was washed with with 10 mL of THF. The outcome solution was quenched with sat. NH₄Cl and extracted with EtOAc. The residue was purified by flash column chromatography (silica, EtOAc). The desired fractions were collected and concentrated in vacuo to yield intermediate (3S)-36 (8.36 g, 53% yield) as a yellow oil.

### Preparation of Intermediate 37

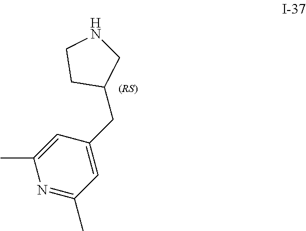

Trifluoroacetic acid (0.31 mL, 4.11 mmol) was added to a solution of intermediate 36 (103 mg, 0.27 mmol) in DCM (0.5 mL) at rt. The mixture was further stirred at rt for 4 h. The volatiles were evaporated under vacuum affording crude intermediate 37 (quantitative).

### Preparation of Intermediate (3S)-37

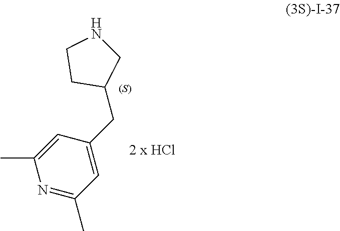

Hydrochloric acid (47.98 mL, 287.91 mmol, 6M in isopropanol) was added to a solution of intermediate (3S)-36 (8.36 g, 28.8 mmol) in MeOH (69.98 mL) at rt. The mixture was further stirred at 50° C. for 1 h. The volatiles were evaporated under vacuum affording crude intermediate (3S)-37 (7.35 g, 97% yileld) as white solid.

### Preparation of Intermediate 38

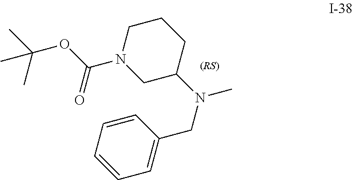

Sodium triacetoxyborohydride (2.38 g, 11.22 mmol) was added to a stirred solution of 1-Boc-3-piperidone (CAS: 98977-36-7; 2 g, 10.04 mmol), N-methylbenzylamine (3.36 mL, 26 mmol), and acetic acid (1.77 mL, 30.96 mmol) in THF (100 mL) at rt. The mixture was further stirred at rt for 18 h. The reaction mixture was quenched with NaHCO₃ (aq. sat. soltn.) and diluted with EtOAc. The organic layer was separated, dried over MgSO₄, filtered and the filtrate was evaporated in vacuo. The residue thus obtained was purified by flash column chromatography (silica, EtOAc in heptane, 0/100 to 30/70). The desired fractions were concentrated in vacuo to yield intermediate 38 as a solid (908 mg, 30% yield).

### Preparation of Intermediate 39

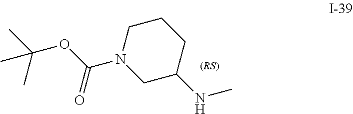

A mixture of intermediate 38 (908 mg, 2.98 mmol) in MeOH (30 mL) and palladium on carbon (95.22 mg; 0.9 mmol) was hydrogenated (atmospheric pressure) at rt for 24 h. The resulting mixture was filtered through a Celite® pad and the filtrate was evaporated in vacuo affording intermediate 39 (633 mg, quantitative).

### Preparation of Intermediate 40

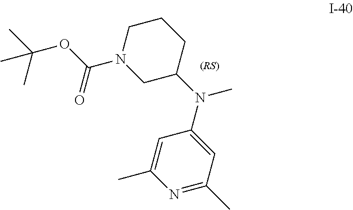

2-Dicyclohexylphosphino-2′-(N,N-dimethylamino)biphenyl (CAS: 213697-53-1; 23.2 mg, 0.059 mmol) was added to a mixture of of intermediate 39 (632 mg, 2.95 mmol), sodium tert-butoxide (567 mg, 5.9 mmol), 4-bromo-2,6-dimethylpyridine (604 mg, 3.24 mmol) and Pd2(dba)₃ (CAS: 51364-51-3; 54 mg, 0.059 mmol) in dry 1,4-dioxane (14.83 mL) at rt while N₂ was bubbled through the reaction mixture. Then resulting mixture was stirred at 100° C. overnight under N₂ atmosphere. The mixture was cooled to rt, diluted with water and extracted with EtOAc. The organic layer was separated, dried over MgSO₄, filtered and the filtrate was evaporated in vacuo. The residue thus obtained was purified by reverse phase chromatography (started: organic phase 10%/aqueous phase 90%; finished: organic phase 46%/aqueous phase 54%. Organic phase: acetonitrile:MeOH 1:1; aqueous phase: 65 mM NH₄OAc:acetonitrile 90:10). The desired fractions were concentrated in vacuo to yield intermediate 40 (102 mg, 10.8% yield).

### Preparation of Intermediate 41

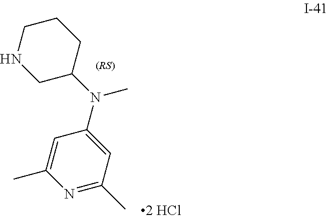

HCl (0.783 mL, 4M solution in 1,4-dioxane) was added to intermediate 40 (100 mg, 0.313 mmol) at rt. The mixture was stirred at rt for 3 h. The volatiles were evaporated under vacuum affording intermediate 41 as a bis-HCl salt (68 mg, 74% yield).

### Preparation of Intermediates 42-110, 119-126, 203 and 224

The following compounds were prepared following a deprotection procedure like the one described for the preparation of intermediate 41 starting from the corresponding Boc-protected amine intermediates using hydrochloric acid or trifluoroacetic acid under standard reaction conditions known to the person skilled in the art. When the procedure for the synthesis of the intermediate is also described in the text, the table also provides alternative conditions.

| | BOC-PROTECTED | |
|---|---|---|
| INTERMEDIATE AMINE | INTERMEDIATE AMINE | ACID/SOLVENT |
| 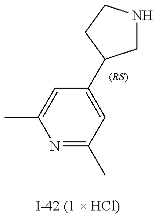 | 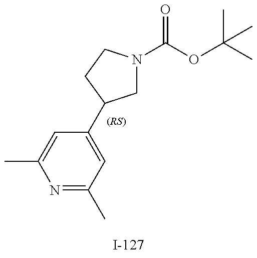 | HCl/1,4-dioxane |
| 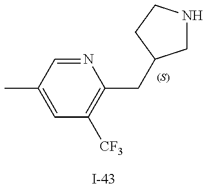 | 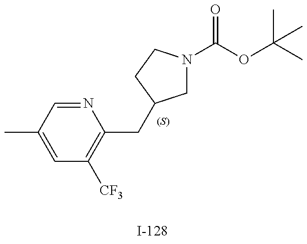 | TFA/DCM |
| 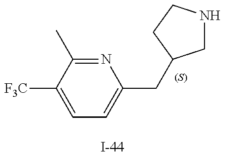 | 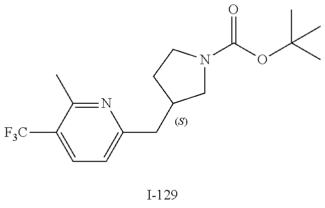 | TFA/DCM |
| 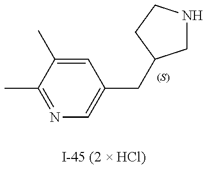 | 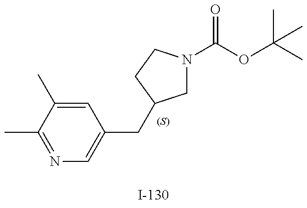 | HCl/1,4-dioxane |
| 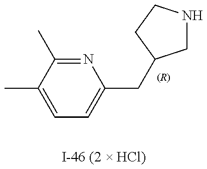 | 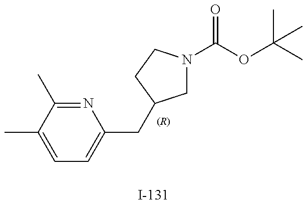 | HCl/1,4-dioxane |
| 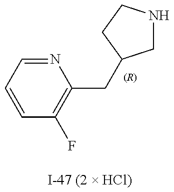 | 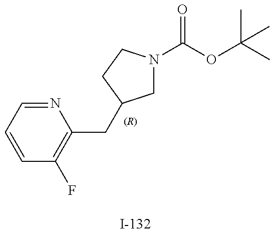 | HCl/1,4-dioxane |
| 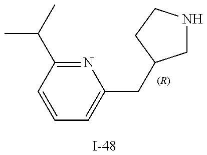 | 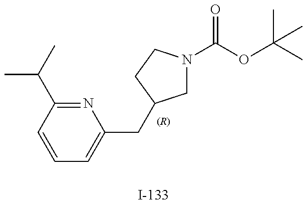 | HCl/1,4-dioxane |
| 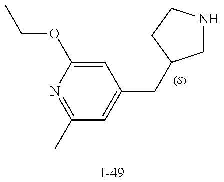 | 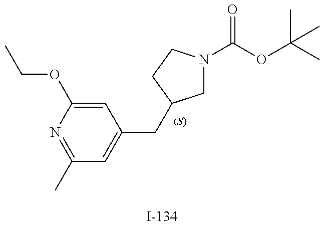 | TFA/DCM |
| 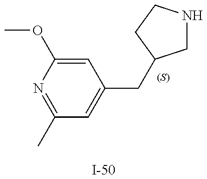 | 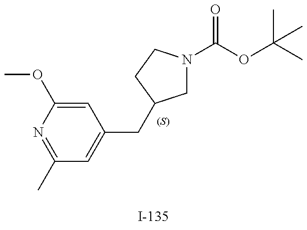 | TFA/DCM |
| 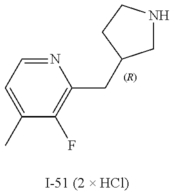 | 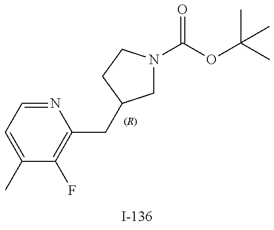 | HCl/1,4-dioxane |
| 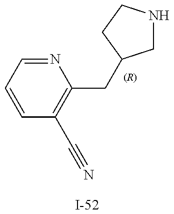 | 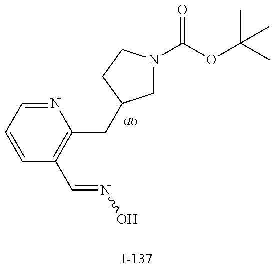 | HCl/1,4-dioxane/ toluene/MeOH |
| 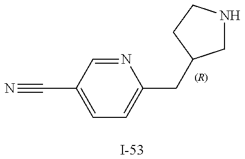 | 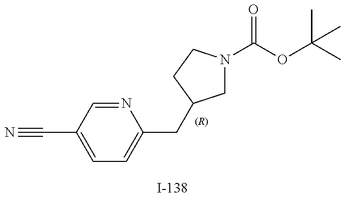 | TFA/DCM |
| 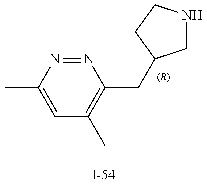 | 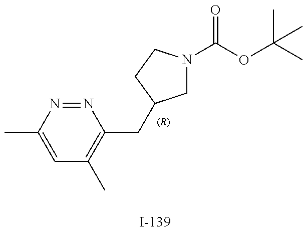 | TFA/DCM |
| 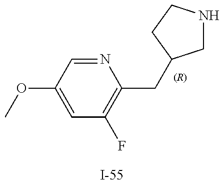 | 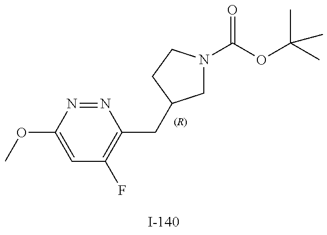 | TFA/DCM |
| 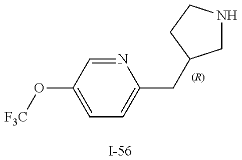 | 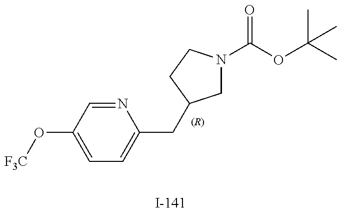 | HCll/1,4-dioxane |
| 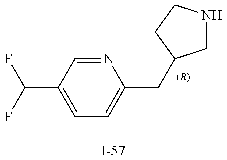 | 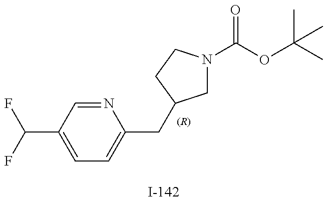 | HCl/1,4-dioxane |
| 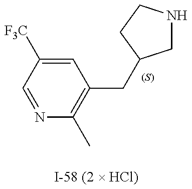 | 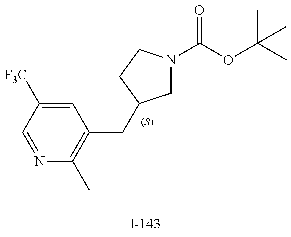 | TFA/DCM |
| 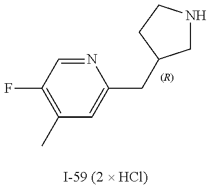 | 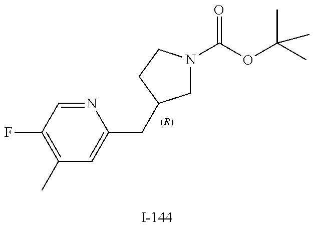 | HCl/1,4-dioxane |
| 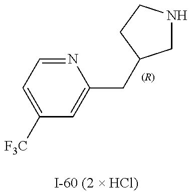 | 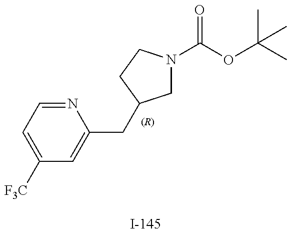 | HCl/1,4-dioxane |
| 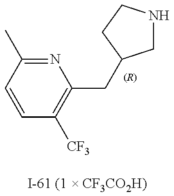 | 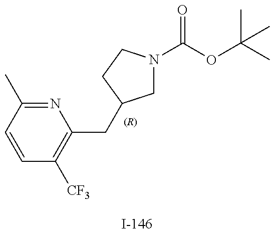 | TFA/DCM |
| 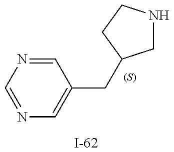 | 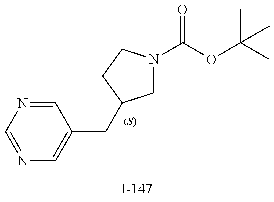 | TFA/DCM |
| 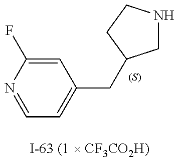 | 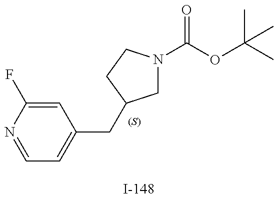 | TFA/DCM |
| 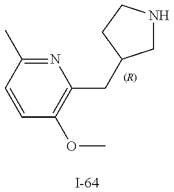 | 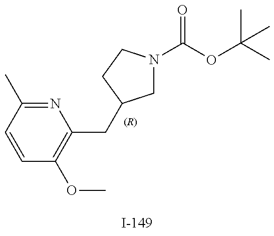 | TFA/DCM |
| 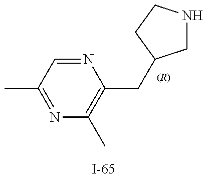 | 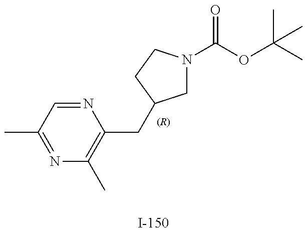 | TFA/DCM |
| 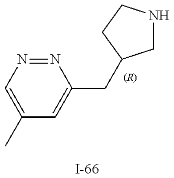 | 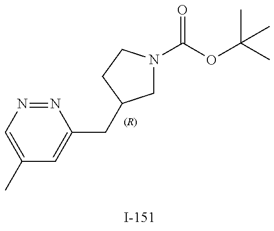 | TFA/DCM |
| 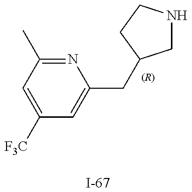 | 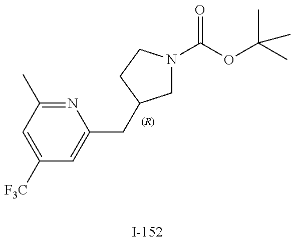 | TFA/DCM |
| 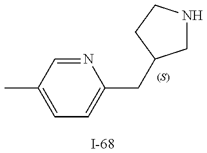 | 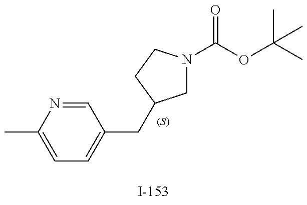 | HCl/MeOH |
| 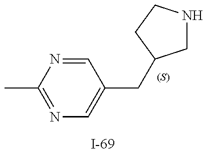 | 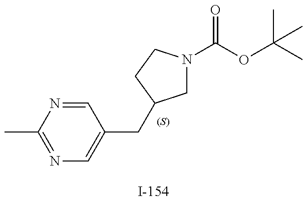 | HCl/MeOH |
| 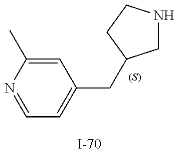 | 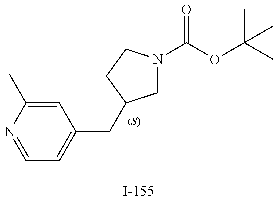 | HCl/MeOH |
| 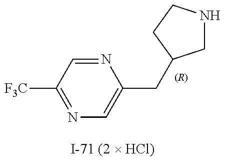 | 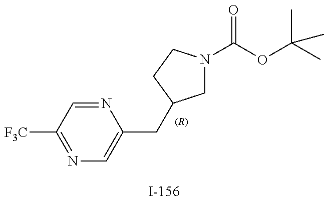 | HCl/1,4-dioxane |
| 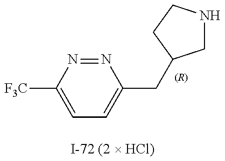 | 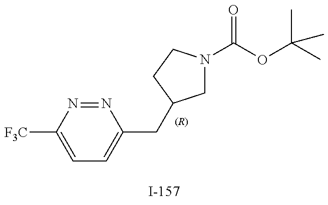 | HCl/1,4-dioxane |
| 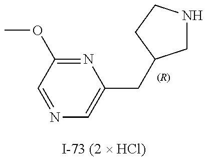 | 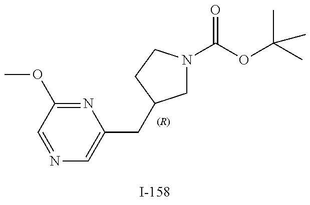 | HCl/1,4-dioxane |
| 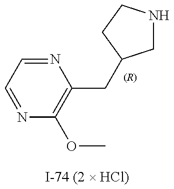 | 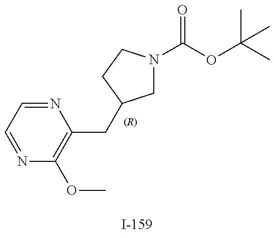 | HCl/1,4-dioxane |
| 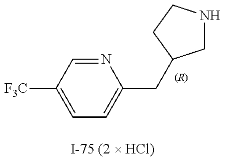 | 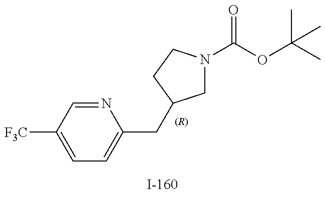 | HCl/1,4-dioxane |
| 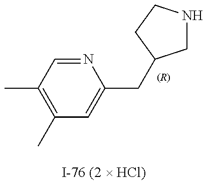 | 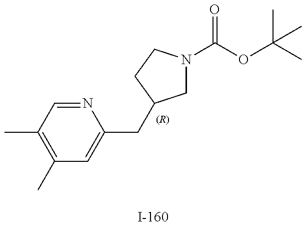 | HCl/1,4-dioxane |
| 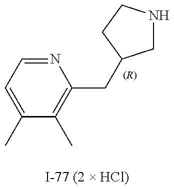 | 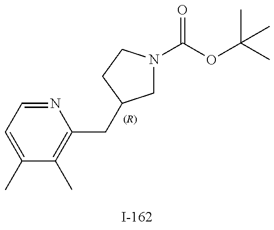 | HCl/1,4-dioxane |
| 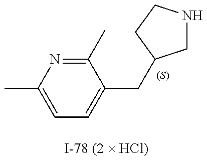 | 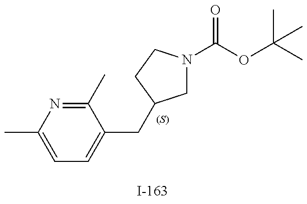 | HCl/1,4-dioxane |
| 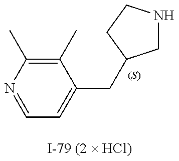 | 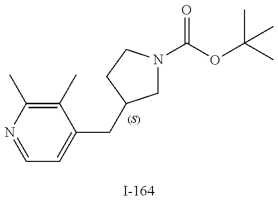 | HCl/1,4-dioxane |
| 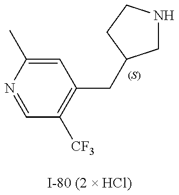 | 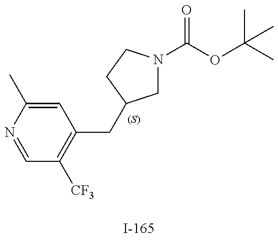 | HCl/1,4-dioxane |
| 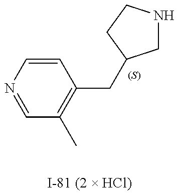 | 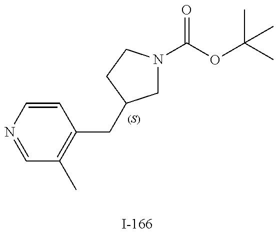 | HCl/1,4-dioxane |
| 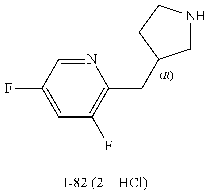 | 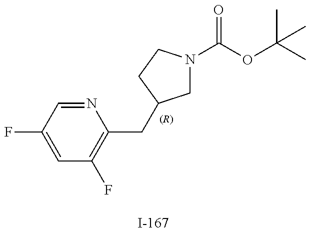 | HCl/1,4-dioxane |
| 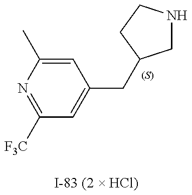 | 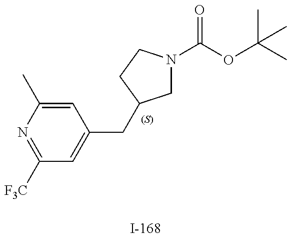 | HCl/1,4-dioxane |
| 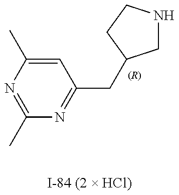 | 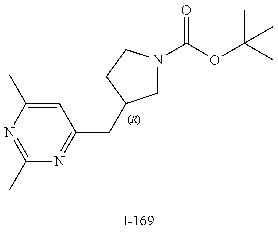 | HCl/1,4-dioxane |
| 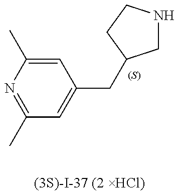 | 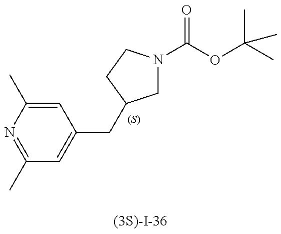 | HCl/iPrOH/MeOH |
| 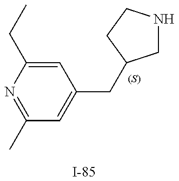 | 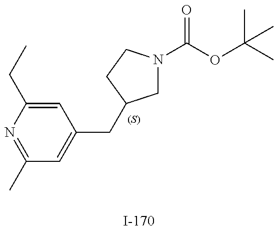 | HCl/1,4-dioxane |
| 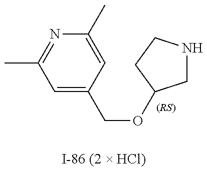 | 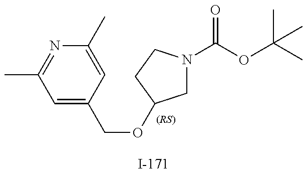 | HCl/iPrOH/MeOH |
| 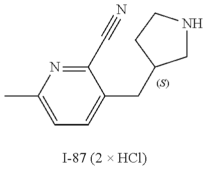 | 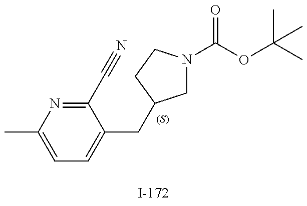 | HCl/1,4-dioxane |
| 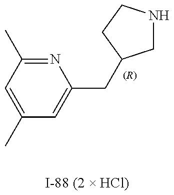 | 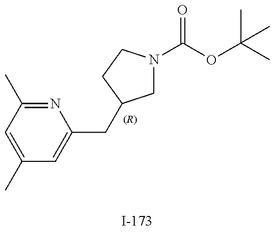 | HCl/1,4-dioxane |
| 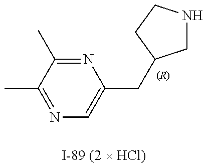 | 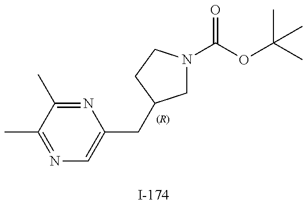 | HCl/1,4-dioxane |
| 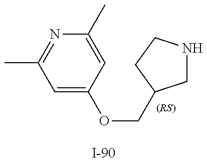 | 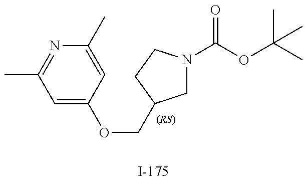 | HCl/iPrOH/MeOH |
| 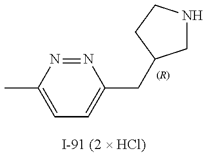 | 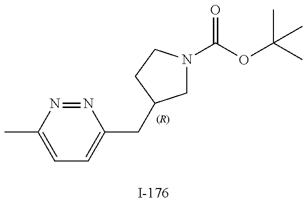 | HCl/1,4-dioxane |
| 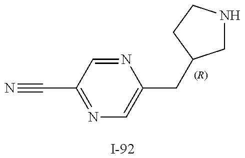 | 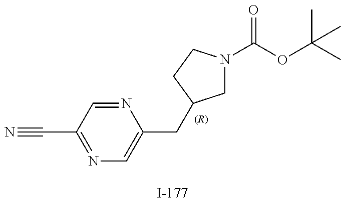 | TFA/DCM |
| 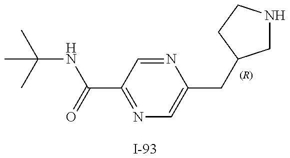 | 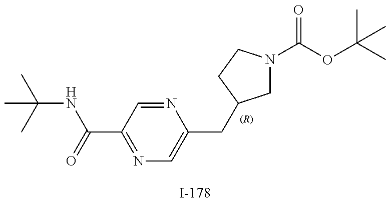 | TFA/DCM |
| 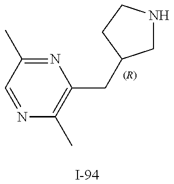 | 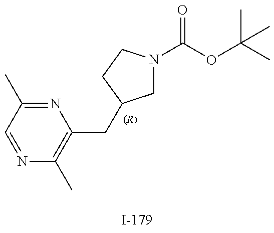 | HCl/1,4-dioxane |
| 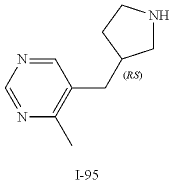 | 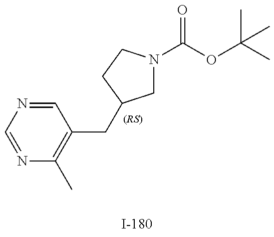 | HCl/1,4-dioxane |
| 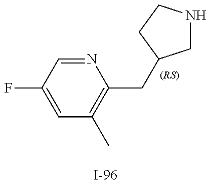 | 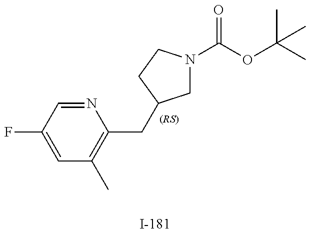 | HCl/1,4-dioxane |
| 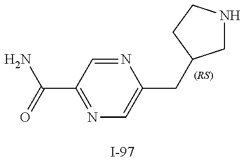 | 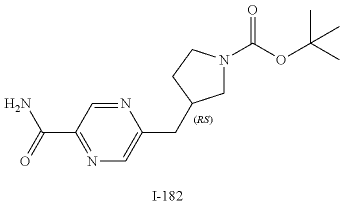 | HCl/1,4-dioxane |
| 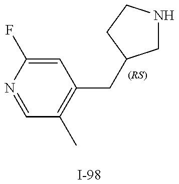 | 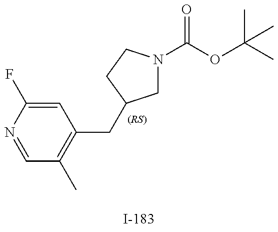 | HCl/1,4-dioxane |
| 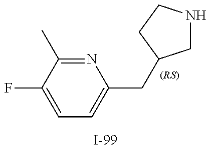 | 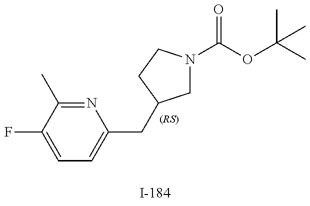 | HCl/1,4-dioxane |
| 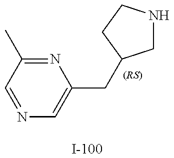 | 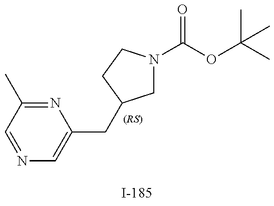 | HCl/1,4-dioxane |
| 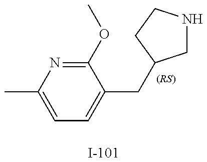 | 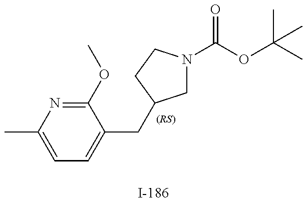 | HCl/1,4-dioxane |
| 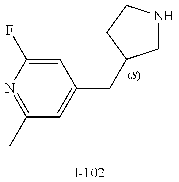 | 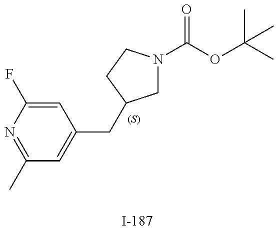 | HCl/1,4-dioxane |
| 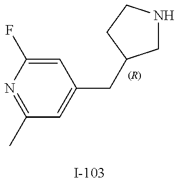 | 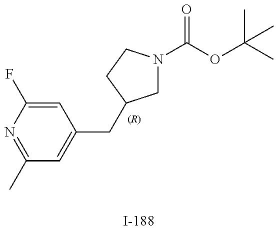 | HCl/1,4-dioxane |
| 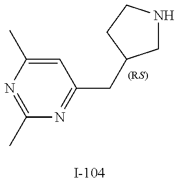 | 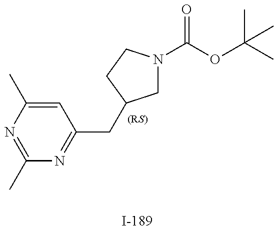 | TFA/DCM |
| 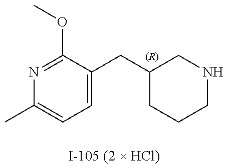 | 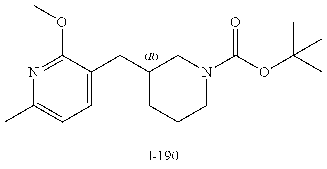 | HCl/1,4-dioxane/2- MeTHF |
| 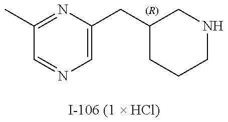 | 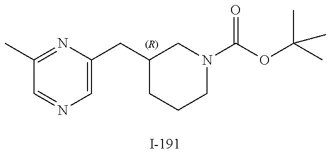 | HCl/1,4-dioxane |
| 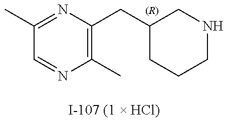 | 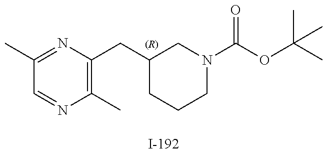 | HCl/1,4-dioxane |
| 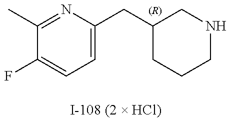 | 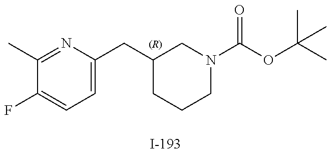 | HCl/1,4-dioxane |
| (3S)-I-23 | (3S)-I-22 | HCl/1,4-dioxane |
| 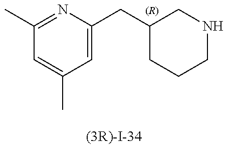 | 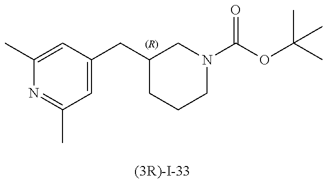 | HCl/iPrOH/MeOH |
| 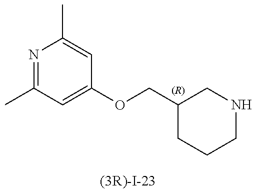 | 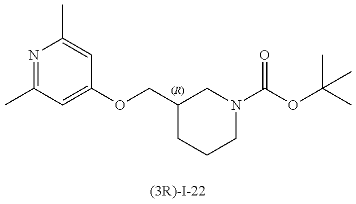 | HCl/1,4-dioxane |
| 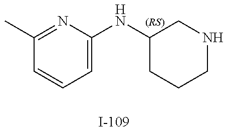 | 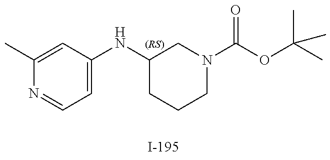 | HCl/1,4-dioxane |
| 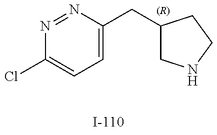 | 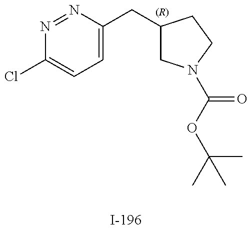 | TFA/DCM |
| 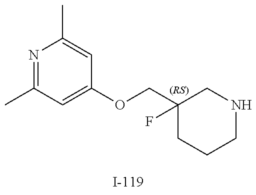 [SMILES: Cc1cc(OCC2(F)CCCNC2)cc(C)n1] | 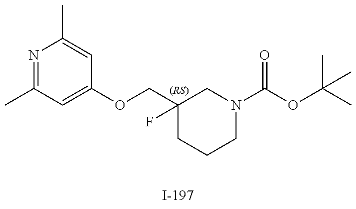 [SMILES: Cc1cc(OCC2(F)CCCN(C(=O)OC(C)(C)C)C2)cc(C)n1] | TFA/DCM |
| 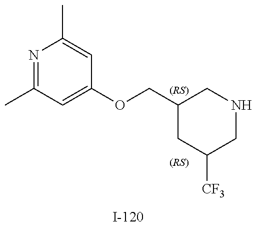 [SMILES: Cc1cc(OCC2CNCC(C(F)(F)F)C2)cc(C)n1] | 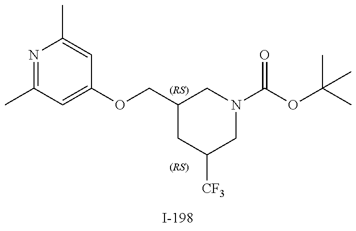 [SMILES: Cc1cc(OCC2CC(C(F)(F)F)CN(C(=O)OC(C)(C)C)C2)cc(C)n1] | HCl/1,4-dioxane |
| 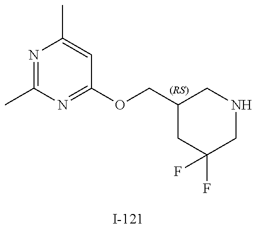 [SMILES: Cc1cc(OCC2CNCC(F)(F)C2)nc(C)n1] | 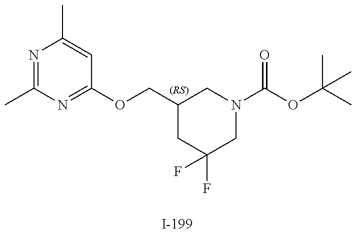 [SMILES: Cc1cc(OCC2CN(C(=O)OC(C)(C)C)CC(F)(F)C2)nc(C)n1] | TFA/DCM |
| 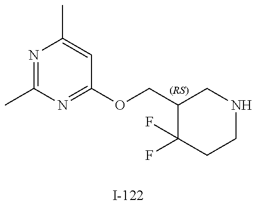 [SMILES: Cc1cc(OCC2CNCCC2(F)F)nc(C)n1] | 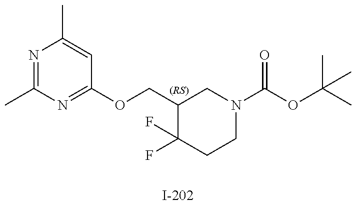 [SMILES: Cc1cc(OCC2CN(C(=O)OC(C)(C)C)CCC2(F)F)nc(C)n1] | TFA/DCM |
| 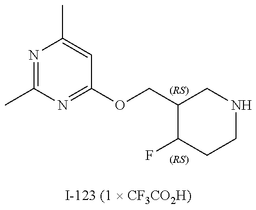 [SMILES: Cc1cc(OCC2CNCCC2F)nc(C)n1] | 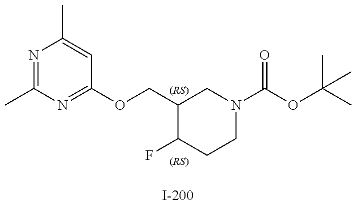 [SMILES: Cc1cc(OCC2CN(C(=O)OC(C)(C)C)CCC2F)nc(C)n1] | TFA/DCM |
| 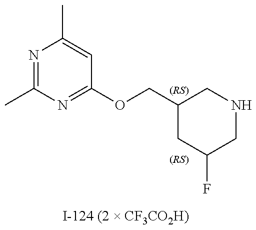 [SMILES: Cc1cc(OCC2CNCC(F)C2)nc(C)n1] | 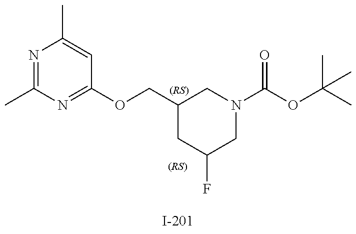 [SMILES: Cc1cc(OCC2CC(F)CN(C(=O)OC(C)(C)C)C2)nc(C)n1] | TFA/DCM |
| 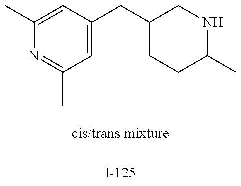 | 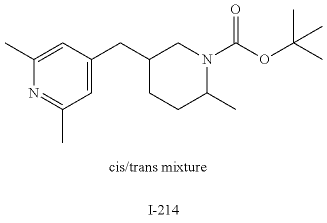 [SMILES: Cc1cc(CC2CCC(C)N(C(=O)OC(C)(C)C)C2)cc(C)n1] | TFA/DCM |
| 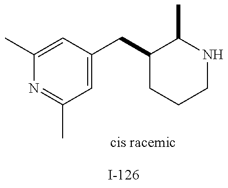 | 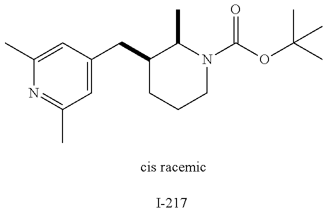 | TFA/DCM |
| 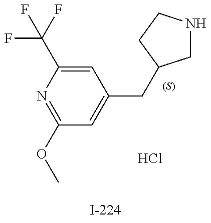 | 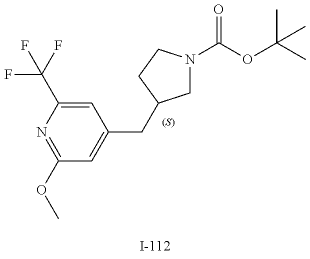 | HCl/1,4-dioxane |
| 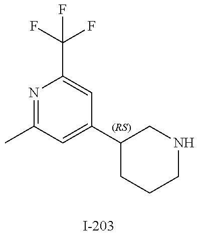 [SMILES: Cc1cc(C2CCCNC2)cc(C(F)(F)F)n1] | 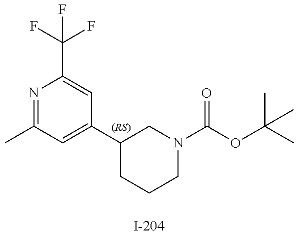 [SMILES: Cc1cc(C2CCCN(C(=O)OC(C)(C)C)C2)cc(C(F)(F)F)n1] | HCl/1,4-dioxane |

### Preparation of Intermediates 128-167, 169-170, 172-174, 176-193, 196, 203, and 208-209

The following compounds were prepared following a reaction procedure like the one described for the preparation of intermediate (3R)-33 starting from the corresponding organozinc intermediates and halo-substituted heteroaromatic intermediates under standard reaction conditions known to the person skilled in the art. When the procedure for the synthesis of the intermediate is also described in the text, the table also provides alternative conditions.

| | | HALO-SUBSTITUTED | |
|---|---|---|---|
| | ORGANOZINC | HETEROAROMATIC | |
| INTERMEDIATE | INTERMEDIATE | INTERMEDIATES | CATALYST/SOLVENT |
| 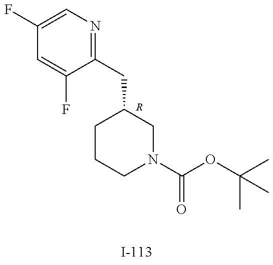 | (3S)-I-30 | CAS: 660425-16-1 | PdCl₂(PPh₃)₂ |
| I-128 | 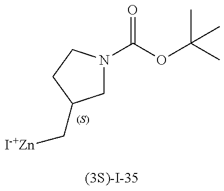 | CAS: 79424-50-3 | (t-Bu₃P)₂Pd |
| I-129 | (3S)-I-35 | CAS: 1023817-24-4 | (t-Bu₃P)₂Pd |
| I-130 | (3S)-I-35 | CAS: 27063-90-7 | (OAc)₂Pd/RuPhos |
| I-131 | (3S)-I-35 | CAS: 99132-28-2 | (OAc)₂Pd/RuPhos |
| I-132 | (3S)-I-35 | CAS: 146141-04-0 | (OAc)₂Pd/RuPhos |
| I-133 | (3S)-I-35 | CAS: 1037223-35-0 | (OAc)₂Pd/RuPhos |
| I-134 | (3S)-I-35 | CAS: 1300633-96-8 | (t-Bu₃P)₂Pd |
| I-135 | (3S)-I-35 | CAS: 1083169-00-9 | (t-Bu₃P)₂Pd |
| I-136 | (3S)-I-35 | CAS: 153035-05-3 | (OAc)₂Pd/RuPhos |
| I-138 | (3S)-I-35 | CAS: 33252-28-7 | (t-Bu₃P)₂Pd |
| I-139 | (3S)-I-35 | CAS: 17258-26-3 | (t-Bu₃P)₂Pd |
| I-140 | (3S)-I-35 | CAS: 1211588-72-5 | (t-Bu₃P)₂Pd |
| I-141 | (3S)-I-35 | CAS: 888327-36-4 | (t-Bu₃P)₂Pd |
| I-142 | (3S)-I-35 | CAS: 1221272-81-6 | (t-Bu₃P)₂Pd |
| I-143 | (3S)-I-35 | CAS: 175227-30-2 | (t-Bu₃P)₂Pd |
| I-144 | (3S)-I-35 | CAS: 881891-83-4 | (t-Bu₃P)₂Pd |
| I-145 | (3S)-I-35 | CAS: 81565-18-6 | (t-Bu₃P)₂Pd |
| I-146 | (3S)-I-35 | CAS: 1099597-74-6 | (t-Bu₃P)₂Pd |
| I-147 | (3S)-I-35 | CAS: 4595-59-9 | (t-Bu₃P)₂Pd |
| I-148 | (3S)-I-35 | CAS: 128071-98-7 | (t-Bu₃P)₂Pd |
| I-149 | (3S)-I-35 | CAS: 24207-22-5 | (t-Bu₃P)₂Pd |
| I-150 | (3S)-I-35 | CAS: 38557-72-1 | (t-Bu₃P)₂Pd |
| I-151 | (3S)-I-35 | CAS: 89283-31-8 | (t-Bu₃P)₂Pd |
| I-152 | (3S)-I-35 | CAS: 22123-14-4 | (t-Bu₃P)₂Pd |
| I-153 | (3S)-I-35 | CAS: 3430-13-5 | Siliacat DPP-Pd |
| I-154 | (3S)-I-35 | CAS: 7752-78-5 | Siliacat DPP-Pd |
| I-155 | (3S)-I-35 | CAS: 3678-62-4 | Siliacat DPP-Pd |
| I-156 | (3S)-I-35 | CAS: 799557-87-2 | (t-Bu₃P)₂Pd |
| I-157 | (3S)-I-35 | CAS: 258506-68-2 | (t-Bu₃P)₂Pd |
| I-158 | (3S)-I-35 | CAS: 33332-30-8 | (t-Bu₃P)₂Pd |
| I-159 | (3S)-I-35 | CAS: 40155-28-0 | (t-Bu₃P)₂Pd |
| I-160 | (3S)-I-35 | CAS: 50488-42-1 | (OAc)₂Pd/RuPhos |
| I-161 | (3S)-I-35 | CAS: 343268-69-9 | (t-Bu₃P)₂Pd |
| I-162 | (3S)-I-35 | CAS: 72093-11-9 | (t-Bu₃P)₂Pd |
| I-163 | (3S)-I-35 | CAS: 2405-06-3 | (t-Bu₃P)₂Pd |
| I-164 | (3S)-I-35 | CAS: 315496-27-6 | (t-Bu₃P)₂Pd |
| I-165 | (3S)-I-35 | CAS: 1804139-74-9 | (OAc)₂Pd/RuPhos |
| I-166 | (3S)-I-35 | CAS: 1681-36-3 | (t-Bu₃P)₂Pd |
| I-167 | (3S)-I-35 | CAS: 660425-16-1 | (OAc)₂Pd/RuPhos |
| I-169 | (3S)-I-35 | CAS: 4472-45-1 | (t-Bu₃P)₂Pd |
| (3S)-I-36 | (3S)-I-35 | CAS: 5093-70-9 | Siliacat DPP-Pd |
| I-170 | (3S)-I-35 | CAS: 155887-27-7 | (t-Bu₃P)₂Pd |
| I-172 | (3S)-I-35 | CAS: 717843-48-6 | (t-Bu₃P)₂Pd |
| I-173 | (3S)-I-35 | CAS: 30838-93-8 | (t-Bu₃P)₂Pd |
| I-174 | (3S)-I-35 | CAS: 59489-32-6 | (t-Bu₃P)₂Pd |
| I-176 | (3S)-I-35 | CAS: 1618-47-9 | (t-Bu₃P)₂Pd |
| I-177 | (3S)-I-35 | CAS: 36070-75-4 | (t-Bu₃P)₂Pd |
| I-178 | (3S)-I-35 | CAS: 36070-75-4 | (t-Bu₃P)₂Pd |
| I-179 | (3S)-I-35 | CAS: 59021-15-7 | (t-Bu₃P)₂Pd |
| I-180 | I-35 | CAS: 1439-09-4 | (t-Bu₃P)₂Pd |
| I-181 | I-35 | CAS: 38186-85-5 | (t-Bu₃P)₂Pd |
| I-182 | I-35 | CAS: 36070-75-4 | (t-Bu₃P)₂Pd |
| I-183 | I-35 | CAS: 153034-94-7 | (t-Bu₃P)₂Pd |
| I-184 | I-35 | CAS: 374633-38-2 | (t-Bu₃P)₂Pd |
| I-185 | I-35 | CAS: 38557-71-0 | (t-Bu₃P)₂Pd |
| I-186 | I-35 | CAS: 717843-47-5 | (t-Bu₃P)₂Pd |
| I-187 | (3S)-I-35 | CAS: 884494-45-5 | Siliacat DPP-Pd |
| I-188 | (3R)-I-35 | CAS: 884494-45-5 | Siliacat DPP-Pd |
| I-189 | I-35 | CAS: 4472-45-1 | Siliacat DPP-Pd |
| I-190 | (3S)-I-30 | CAS: 717843-47-5 | (t-Bu₃P)₂Pd |
| I-191 | (3S)-I-30 | CAS: 38557-71-0 | (t-Bu₃P)₂Pd |
| I-192 | (3S)-I-30 | CAS: 95-89-6 | (t-Bu₃P)₂Pd |
| I-193 | (3S)-I-30 | CAS: 374633-38-2 | (t-Bu₃P)₂Pd |
| I-196 | (3S)-I-35 | CAS: 141-30-0 | (t-Bu₃P)₂Pd |
| 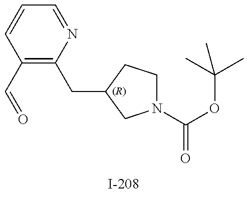 | (3S)-I-35 | CAS: 36404-88-3 | (t-Bu₃P)₂Pd |
| 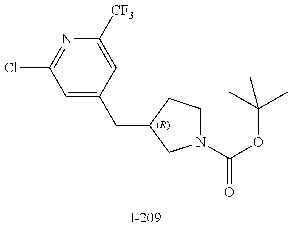 | (3S)-I-35 | CAS: 205444-22-0 | (t-Bu₃P)₂Pd |

### Preparation of Intermediate 111

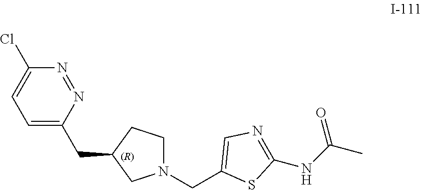

Sodium triacetoxyborohydride (21.9 mg, 0.1 mmol) was added to a stirred solution of intermediate 110 (17 mg, 0.086 mmol) and intermediate 12 (14.6 mg, 0.086 mmol) in DCM (0.48 mL). The mixture was stirred at rt for 6 h. The mixture was concentrated in vacuo. The resultant oil was purified by flash column chomatography (silica; 7M solution of amonia in methanol in DCM 0/100 to 05/95). The desired fractions were collected and concentrated in vacuo to yield intermediate 111 as a pale yellow solid (20 mg, 85% pure, 55% yield).

### Preparation of Intermediate 118

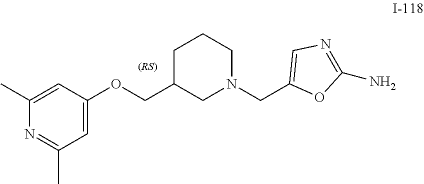

Then the mixture was concentrated in vacuo and the residue purified by flash column chromatography (SiO₂, MeOH in DCM from 0/100 to 100/0). The desired fractions were collected and concentrated in vacuo to yield intermediate 118 (106 mg, 80% yield).

### Preparation of Intermediate 127

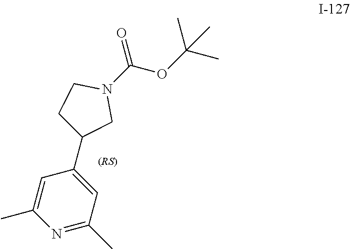

Intermediate I-127 was prepared following the same reaction procedure as for the preparation of intermediate I-10 but starting from intermediate I-207.

### Preparation of Intermediate 137

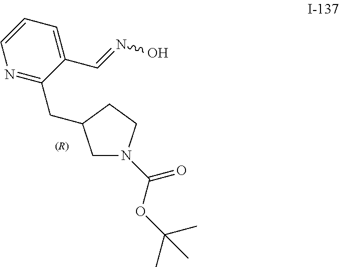

Hydroxylamine hydrochloride (50.6 mg, 0.73 mmol) was added to a stirred solution of intermediate 208 (223 mg, 0.56 mmol, 73% pure) and sodium acetate trihydrate (229 mg, 1.68 mmol) in MeOH (5 mL). The mixture was stirred at rt for 1 h. Then the solvent was evaporated in vacuo and the residue was washed several times with EtOAc filtered and concentrated in vacuo to yield intermediate 137 (202 mg, 76% yield, 65% pure) as a brown solid.

### Preparation of Intermediate 168

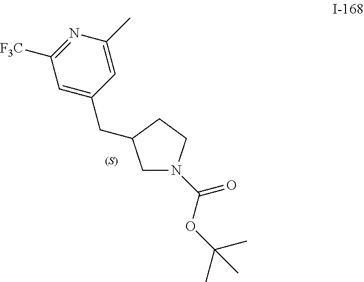

Potassium carbonate (0.13 g, 0.94 mmol) was added to a stirred solution of intermediate 209 (172 mg, 0.47 mmol) in 1,4-dioxane (1.38 mL) and it was deoxygenated with a N₂ flow for 5 min. Then, trimethylboroxine (0.119 mg, 0.85 mmol), (OAc)₂Pd (5.3 mg, 0.023 mmol) and tricyclohexylphosphine tetrafluoroborate (CAS: 17.4 mg, 0.047 mmol) were added. The mixture was stirred at 100° C. for 2 h under N₂ atmosphere. After cooling to rt, the mixture was washed with H₂O and extracted with DCM. The organic layer was separated, dried (MgSO₄), filtered and the solvents evaporated in vacuo. The crude product was purified by flash column chromatography (silica; EtOAc in heptane: 0/100 to 15/85). The desired fractions were collected and concentrated in vacuo to yield intermediate 168 (140.6 mg, 86%) as pale yellow oil.

### Preparation of Intermediate 171

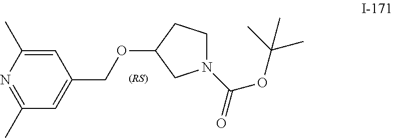 [SMILES: Cc1cc(COC2CCN(C(=O)OC(C)(C)C)C2)cc(C)n1]

Intermediate I-171 was prepared following the same reaction procedure as for the preparation of intermediate I-24 but starting from 1-boc-3-pyrrolidinol.

### Preparation of Intermediate 175

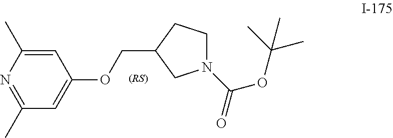 [SMILES: Cc1cc(OCC2CCN(C(=O)OC(C)(C)C)C2)cc(C)n1]

Diisopropyl azodicarboxylate (1.2 g, 5.96 mmol) was added to a stirred solution of tert-butyl-3-(hydroxymethyl)pyrrolidine-1-carboxylate (CAS: 114214-69-6; 400 mg, 2 mmol), 2,6-dimethyl-4-hydroxypyridine (367 mg, 2.98 mmol) and triphenylphosphine (1.56 g, 5.96 mmol) in acetonitrile (12.4 mL) at rt. The mixture was stirred at 65° C. for 16 h. The mixture was concentrated in vacuo and the residue was purified by flash column chromatography (SiO₂; EtOAc in Heptane from 0:100 to 100/0). The desired fractions were collected and concentrated in vacuo to yield a solid that was further purified by ion exchange chromatography (ISOLUTE® SCX2 eluting with MeOH and 7N ammonia solution in MeOH). The desired fraction was collected and concentrated in vacuo to yield intermediate 175 (238 mg, 37%) as a clear yellow oil.

### Preparation of Intermediate 194

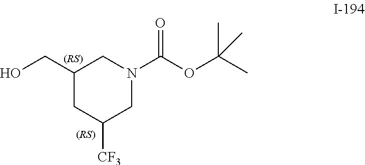 [SMILES: CC(C)(C)OC(=O)N1CC(CO)CC(C(F)(F)F)C1]

To a solution of intermediate 210 (0.797 mg, 2.56 mmol) in EtOH (8.3 mL) at 0° C. was added sodium cyanoborohydride (0.329 g, 8.7 mmol) in 3 lots over 30 min. After completion of addition, the reaction mixture was stirred for 30 min at rt. The volatiles were evaporated under reduced pressure, and NaHCO₃ sat. was added (10 mL) and the mixture extracted with EtOAc (20 mL). The organic layer was dried over MgSO₄ and filtered. The solvent was concentrated in vacuo. The crude material was purified by flash cromatography (SiO₂, EtOAc in heptane 0/100 to 100/0). The desired fractions were collected and concentrated in vacuo to yield intermediate 194 (980 mg, 98% yield, 73% pure) as a colourless oil.

### Preparation of Intermediate 195

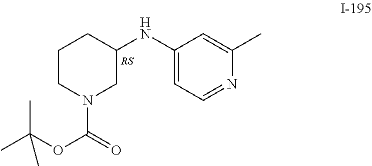 [SMILES: Cc1cc(NC2CCCN(C(=O)OC(C)(C)C)C2)ccn1]

Intermediate 195 was prepared from tert-butyl 3-aminopiperidine-1-carboxylate following the same reaction procedure that the one for the preparation of intermediate 26.

### Preparation of Intermediate 197

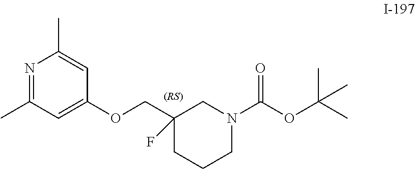 [SMILES: Cc1cc(OCC2(F)CCCN(C(=O)OC(C)(C)C)C2)cc(C)n1]

Intermediate 197 was prepared from 4-bromo-2,6-dimethylpyridine and 1-piperidinecarboxylic acid, 3-fluoro-3-(hydroxymethyl)-1,1-dimethylethyl ester (CAS: 1209781-11-2) following the same reaction procedure that the one for the preparation of intermediate 22 and using potassium tert-butoxyde as base and THF as solvent.

### Preparation of Intermediate 198

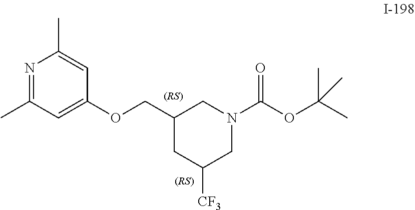 [SMILES: Cc1cc(OCC2CC(C(F)(F)F)CN(C(=O)OC(C)(C)C)C2)cc(C)n1]

Intermediate 198 was prepared from intermediate 194 following the same reaction procedure that the one for the preparation of intermediate 175.

### Preparation of Intermediate 199

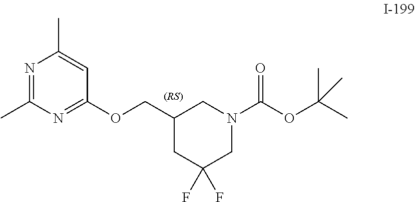 [SMILES: Cc1cc(OCC2CN(C(=O)OC(C)(C)C)CC(F)(F)C2)nc(C)n1]

Diethylaminosulfur trifluoride (0.238 mL, 1.9 mmol) was added to a solution of intermediate 211 (131 mg, 0.4 mmol) in anhydrous DCM (2.9 MmL) at 0° C. The mixture was stirred at rt for 16 h. The mixture was diluted with NaHCO₃ (aq. Sat. soltn.) and extracted with DCM. The organic layer was separated, dried (MgSO₄), filtered and the solvents evaporated in vacuo. The crude product was purified by flash column chromatography (silica; EtOAc in heptane 0/100 to 50/50). The desired fractions were collected and concentrated in vacuo to yield intermediate 199 (55 mg, 39% yield) as a colourless oil

### Preparation of Intermediate 200

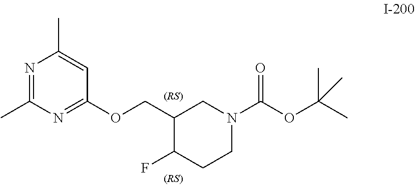 [SMILES: Cc1cc(OCC2CN(C(=O)OC(C)(C)C)CCC2F)nc(C)n1]

Intermediate 200 was prepared from intermediate 213 following the same reaction procedure that the one for the preparation of intermediate 199.

### Preparation of Intermediate 201

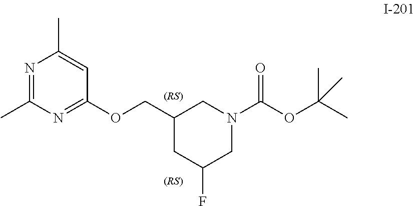 [SMILES: Cc1cc(OCC2CC(F)CN(C(=O)OC(C)(C)C)C2)nc(C)n1]

Intermediate 201 was prepared from intermediate 212 following the same reaction procedure as the one for the preparation of intermediate 199.

### Preparation of Intermediate 202

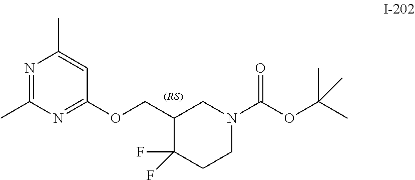 [SMILES: Cc1cc(OCC2CN(C(=O)OC(C)(C)C)CCC2(F)F)nc(C)n1]

Intermediate 202 was prepared from intermediate 223 following the same reaction procedure as the one for the preparation of intermediate 199.

### Preparation of Intermediate 207

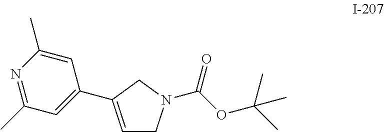 [SMILES: Cc1cc(C2=CCN(C(=O)OC(C)(C)C)C2)cc(C)n1]

Intermediate I-207 was prepared following the same reaction procedure as for the preparation of intermediate I-9 but starting from 4-bromo-2,6-dimethylpyridine and CAS: 212127-83-8.

### Preparation of Intermediate 210

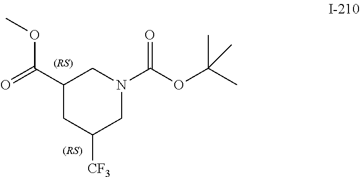 [SMILES: COC(=O)C1CC(C(F)(F)F)CN(C(=O)OC(C)(C)C)C1]

Di-tert-butyl dicarbonate (2 mL, 8.7 mmol) was added to a mixture of methyl 5-(trifluoromethyl)piperidine-3-carboxylate (CAS: 1269755-53-4; 2.3 g, 8.7 mmol) and triethylamine (2.42 mL, 17.43 mmol) in DCM (40 mL) at rt. The mixture was stirred at rt overnight. Water was added and the mixture was extracted with EtOAc. The organic layer was washed with NaHCO₃ (aq. sat. soltn.), dried over MgSO₄, filtered and concentrated in vacuo. The crude material was purified by flash cromatography (SiO₂, EtOAc in heptane 0/100 to 15/85). The desired fractions were collected and concentrated in vacuo to yield intermediate 210 (797 mg, 80% pure).

### Preparation of Intermediate 211

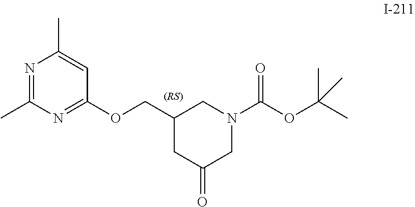 [SMILES: Cc1cc(OCC2CC(=O)CN(C(=O)OC(C)(C)C)C2)nc(C)n1]

Dess-Martin periodinane (241 mg, 0.56 mmol) was added to a stirred solution of intermediate 212 (160 mg, 0.474 mmol) in DCM (10 mL) at 0° C. The mixture was stirred at rt for 20 h. The mixture was diluted with NaHCO₃ (aq. sat. soltn.) and stirred for 30 min at rt. The mixture was extracted with DCM. The organic layer was separated, dried (MgSO₄), filtered and the solvents evaporated in vacuo. The crude product was purified by flash column chromatography (silica; MeOH/DCM (1:10) in DCM 0/100 to 40/60). The desired fractions were collected and concentrated in vacuo to yield intermediate 211 (130 mg, 82% yield) as a colourless sticky solid.

### Preparation of Intermediate 212

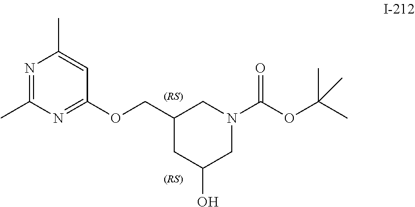 [SMILES: Cc1cc(OCC2CC(O)CN(C(=O)OC(C)(C)C)C2)nc(C)n1]

Potassium tert-butoxide (130 mg, 1.16 mmol) was added to a stirred solution of 3-hydroxy-5-(hydroxymethyl)-1-piperidinecarboxylic acid 1,1-dimethylethyl ester (CAS: 955029-43-3; 256 mg, 1.1 mmol) in DMF (10 mL) under nitrogen at rt. The mixture was stirred at rt for 40 min. Then, a solution of 4-chloro-2,6-dimethylpyrimidine (158 mg, 1.1 mmol) in DMF (5 mL) was added dropwise. The mixture was stirred at rt for 18 h. The mixture was diluted with water and extracted with EtOAc The organic layer was separated, dried (MgSO₄), filtered and the solvents evaporated in vacuo. The crude product was purified by flash column chromatography (silica; EtOAc in heptane 0/100 to 100/0). The desired fractions were collected and concentrated in vacuo to yield intermediate 212 (160 mg, 33% yield, 78% pure) as a colourless oil.

### Preparation of Intermediate 213

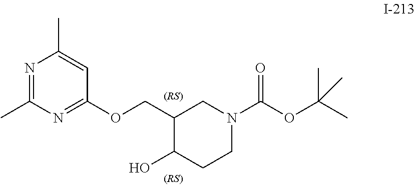 [SMILES: Cc1cc(OCC2CN(C(=O)OC(C)(C)C)CCC2O)nc(C)n1]

Intermediate 213 was prepared from 4-hydroxy-3-(hydroxymethyl)-1-piperidinecarboxylic acid 1,1-dimethylethyl ester (CAS 849767-19-7) following the same reaction procedure that the one for the preparation of intermediate 212.

### Preparation of Intermediate 214

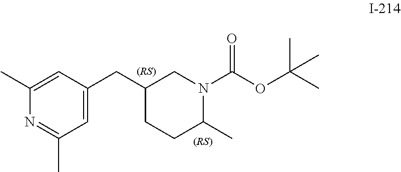 [SMILES: Cc1cc(CC2CCC(C)N(C(=O)OC(C)(C)C)C2)cc(C)n1]

Intermediate I-214 was prepared following the same reaction procedure as for the preparation of intermediate (3R)—I-33 but starting from 4-bromo-2,6-dimethylpyridine and intermediate I-215.

### Preparation of Intermediate 215

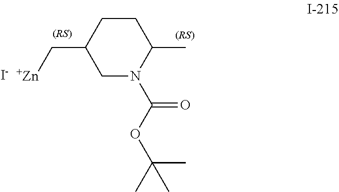

Intermediate I-215 was prepared following the same reaction procedure as for the preparation of intermediate (3S)—I-30 but starting from intermediate I-216.

### Preparation of Intermediate 216

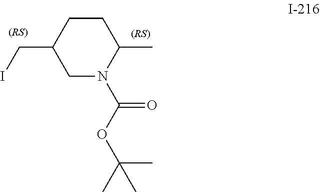 [SMILES: CC1CCC(CI)CN1C(=O)OC(C)(C)C]

To a solution of 1-piperidinecarboxylic acid, 5-(hydroxymethyl)-2-methyl-, 1,1-dimethylethyl ester (CAS: 278789-38-1; 1.2 g, 5.23 mmol) in DCM (72 mL), methyl iodide (2.92 g, 11.5 mmol) and triphenylphosphine (3 g, 11.51 mmol) were added. The reaction mixture was stirred at rt 30 min, then imidazole (0.93 g, 13.6 mmol) was added in one portion and the resulting solution heated to reflux and stirred at reflux for 3 h. After cooling, the reaction mixture was diluted with DCM (1×20 mL) and the organic phase washed with sodium thiosulfate (1×10 mL of a 5% aqueous solution) and brine (1×5 mL). The separated organic phase was then dried (MgSO₄), filtered and concentrated under reduced pressure to give a yellow oil. The crude was purified by flash column chromatography (silica; EtOAc in heptane 0/100 to 10/90). The desired fractions were collected and evaporated in vacuo to afford intermediate 216 (1.2 g, 68% yield) as a yellow oil.

### Preparation of Intermediate 217

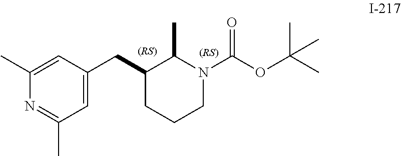

Intermediate I-217 was prepared following the same reaction procedure as for the preparation of intermediate (3R)—I-33 but starting from 4-bromo-2,6-dimethylpyridine and intermediate I-218.

### Preparation of Intermediate 218

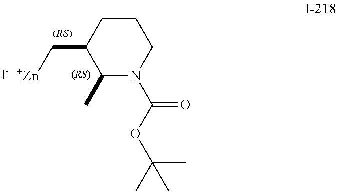

Intermediate I-218 was prepared following the same reaction procedure as for the preparation of intermediate (3S)—I-30 but starting from intermediate I-219.

### Preparation of Intermediate 219

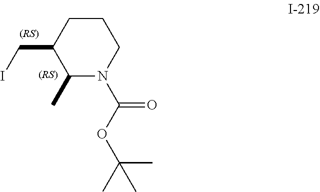 [SMILES: C[C@H]1[C@@H](CI)CCCN1C(=O)OC(C)(C)C]

Intermediate I-219 was prepared following the same reaction procedure as for the preparation of intermediate 216 but starting from intermediate I-220.

### Preparation of Intermediate 220

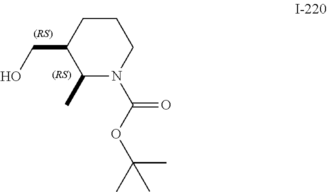 [SMILES: C[C@H]1[C@@H](CO)CCCN1C(=O)OC(C)(C)C]

To a solution of 2-methyl-1,3-piperidinedicarboxylic acid 1-(1,1-dimethylethyl) 3-methyl ester (CAS: 2111567-11-2; 1.75 g, 6.8 mmol) in THF (40 mL), lithium aluminium hydride (10.2 mL, 10.2 mmol, 1M solution in THF) was added at −78° C. After stirring at 0° C. for 30 min, the reaction mixture was quenched dropwise with water (10 mL) at −78° C. The mixture was warmed at rt and then treated with water, and the crude was extracted with EtOAc. The phases were separated and the combined organic extracts were washed with brine, dried (Na₂SO₄), filtered and concentrated under reduced pressure to afford intermediate 220 (1.5 g, 96% yield) as an oil.

### Preparation of Intermediate 221

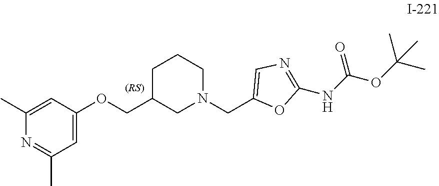 [SMILES: Cc1cc(OCC2CCCN(Cc3cnc(NC(=O)OC(C)(C)C)o3)C2)cc(C)n1]

Lithium aluminium hydride (33.6 mg, 0.89 mmol) was added to a stirred suspension of intermediate 222 (136.8 mg, 0.3 mmol) in anhydrous THF (20 mL). The mixture was stirred at 60° C. for 4 h. The reaction treated with ice, and then NaOH 1N (4 mL) and EtOAc were added. The reaction mixture was extracted with EtOAc. The organic layer was separated, dried (MgSO₄), filtered and the solvents evaporated in vacuo. The crude product was purified by flash column chromatography (silica; MeOH/NH₃ in DCM 0/100 to 100/0). The desired fractions were collected and concentrated in vacuo to yield a residue that was further purified by reverse phase chromatography (59% [25 mM NH₄HCO₃]-41% [ACN:MeOH 1:1] to 17% [25 mM NH₄HCO₃]-83% [ACN:MeOH 1:1]). The desired fractions were collected and concentrated in vacuo to yield intermediate 221 (36 mg, 29% yield).

### Preparation of Intermediate 222

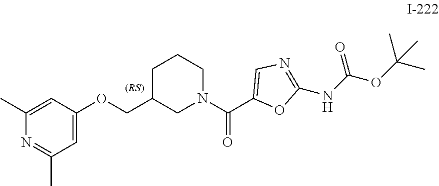 [SMILES: Cc1cc(OCC2CCCN(C(=O)c3cnc(NC(=O)OC(C)(C)C)o3)C2)cc(C)n1]

To a solution of 2-(tert-butoxycarbonylamino)oxazole-5-carboxylic acid (CAS: 903094-60-0; 119.6 mg, 0.52 mmol) in DCM (8 mL) at 0° C. was added triethylamine (0.21 mL, 1.5 mmol) and intermediate 23 (110 mg, 0.5 mmol). The reaction mixture was stirred at 0° C. for 15 min and then 1-propanephosphonic acid cyclic anhydride (0.6 mL, 1 mmol) was added. The reaction mixutre was allowed to warm to rt and then it was further stirred for 14 h. The reaction mixture was concentrated under reduced pressure. DCM and water were added. The organic phase was dried over MgSO₄, filtered and concentrated under reduce pressure. The crude product was purified by flash column chromatography (silica; MeOH/NH₃/DCM in DCM 0/100 to 100/0). The desired fractions were collected and concentrated in vacuo to yield intermediate 222 (159 mg, 74% yield).

### Preparation of Intermediate 223

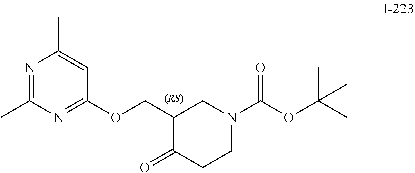 [SMILES: Cc1cc(OCC2CN(C(=O)OC(C)(C)C)CCC2=O)nc(C)n1]

Intermediate 223 was from intermediate 213 following the same reaction procedure that the one for the preparation of intermediate 211.

### Preparation of Intermediate 112

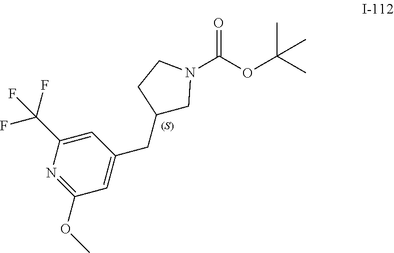

Intermediate 209 (350 mg, 0.96 mmol) was dissolved in a solution of sodium methoxide in dry MeOH (1.22 mL, 0.96 mmol) and stirred at rt for 16 h. Then water was added and the desired product extracted with DCM. The organic layer was separated, dried (Na₂SO₄), filtered and the solvent evaporated in vacuo to yield intermediate 112 (250 mg, 72% yield) as a colorless oil.

### Preparation of Intermediate 204

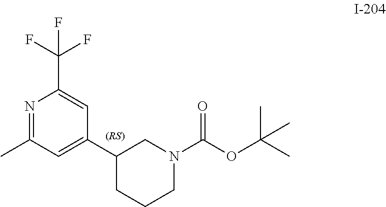 [SMILES: Cc1cc(C2CCCN(C(=O)OC(C)(C)C)C2)cc(C(F)(F)F)n1]

A solution of intermediate 205 (980 mg, 2.86 mmol) in EtOH (56.4 mL) was hydrogenated in a H-cube (Pd/C 10%, full H₂, rt, 1 mL/min). The solvent was evaporated to yield intermediate 204 (800 mg, 81% yield) as a colorless oil that crystallized upon standing and was used in the next step without further purification.

### Preparation of Intermediate 205

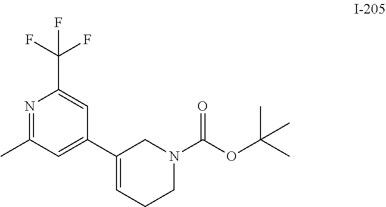 [SMILES: Cc1cc(C2=CCCN(C(=O)OC(C)(C)C)C2)cc(C(F)(F)F)n1]

Intermediate I-205 was prepared following the same reaction procedure as for the preparation of intermediate I-168 but starting from intermediate 206.

### Preparation of Intermediate 206

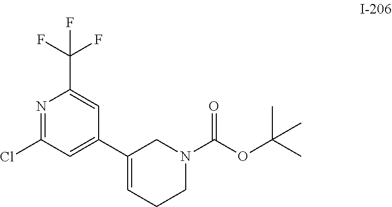 [SMILES: CC(C)(C)OC(=O)N1CCC=C(c2cc(Cl)nc(C(F)(F)F)c2)C1]

Intermediate I-206 was prepared following the same reaction procedure as for the preparation of intermediate I-10 but starting from 2-chloro-4-iodo-6-trifluoromethylpyridine (CAS: 1251537-34-4).

### Preparation of Intermediate 225

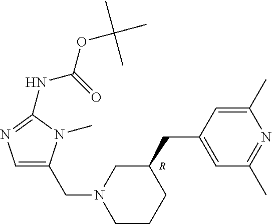

Sodium triacetoxyborohydride (80 mg, 0.38 mmol) was added to a stirred solution of -(3R)—I-34 (46.3 mg, 0.23 mmol) and N-(5-formyl-1-methyl-1H-imidazol-2-yl)-carbamic acid 1,1-dimethylethyl ester ([1520189-43-8], 51 mg, 0.23 mmol) in DCM (1.1 mL) in a sealed tube and under N₂. The mixture was stirred at rt for 16 h. Then the mixture was treated with sat. NaHCO₃ and extracted with DCM. The organic layer was separated, dried (MgSO₄), filtered and the solvents evaporated in vacuo. The crude product was purified by flash column chromatography (SiO₂, 7N solution of NH₃ in MeOH in DCM 0/100 to 5/95). The desired fractions were collected and concentrated in vacuo to yield intermediate 225 (65 mg, 69%) as a yellow oil.

### B. PREPARATION OF FINAL COMPOUNDS

### E1. Preparation of Product 1

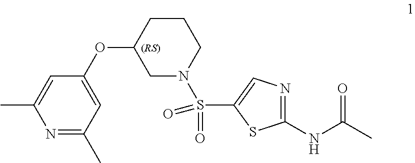 [SMILES: CC(=O)Nc1ncc(S(=O)(=O)N2CCCC(Oc3cc(C)nc(C)c3)C2)s1]

2-Acetylamino-thiazole-5-sulfonyl chloride (CAS: 654072-71-6, 43 mg, 0.18 mmol) was added portion wise to a stirred solution of intermediate 2 (50 mg, 0.18 mmol, bis HCl salt) and diisopropylethylamine (0.09 mL, 0.57 mmol) in DCM (7.8 mL) at 0° C. and the mixture was further stirred at 0° C. for 1 h. NaHCO₃ (aq. sat. soltn.) was added and the organic layer was separated dried over MgSO₄, filtered and evaporated under vacuum. The solid thus obtained was washed with Et₂O and then it was dried in the vacuum oven (50° C.) affording product 1 as a white solid (26 mg, 35% yield).

### E2. Preparation of Product 2

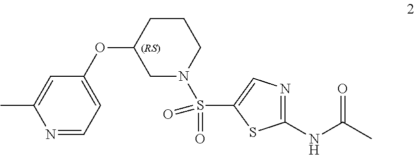 [SMILES: CC(=O)Nc1ncc(S(=O)(=O)N2CCCC(Oc3ccnc(C)c3)C2)s1]

2-Acetylamino-thiazole-5-sulfonyl chloride (CAS: 654072-71-6, 45 mg, 0.19 mmol) was added portion wise to a stirred solution of intermediate 4 (50 mg, 0.19 mmol, bis HCl salt) and diisopropylethylamine (0.1 mL, 0.6 mmol) in DCM (8.2 mL) at 0° C. and the mixture was further stirred at 0° C. for 1 h. NaHCO₃ (aq. sat. soltn.) was added and the organic layer was separated dried over MgSO₄, filtered and evaporated under vacuum. The solid thus obtained was washed with Et₂O and then it was dried in the vacuum oven (50° C.) affording product 2 as a white solid (62.9 mg, 92% yield).

### E.3 Preparation of Product 3

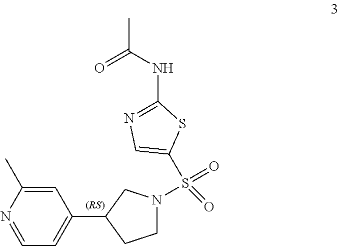 [SMILES: CC(=O)Nc1ncc(S(=O)(=O)N2CCC(c3ccnc(C)c3)C2)s1]

2-Acetylamino-thiazole-5-sulfonyl chloride (CAS: 654072-71-6, 69 mg, 0.28 mmol) was added to a stirred solution of intermediate 6 (67 mg, 0.28 mmol, bis HCl salt) and diisopropylethylamine (0.19 mL, 1.14 mmol) in DCM (2.5 mL) at rt and the mixture was further stirred at rt for 16 h. DCM and NaHCO₃ (aq. sat. soltn.) were added and the organic layer was separated dried over MgSO₄, filtered and evaporated under vacuum. The solid thus obtained was triturated with EtOAc/diisopropylether/MeOH affording product 3 as an off white solid (51 mg, 49% yield).

### E4. Preparation of Product 4

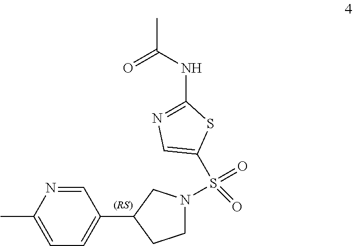 [SMILES: CC(=O)Nc1ncc(S(=O)(=O)N2CCC(c3ccc(C)nc3)C2)s1]

2-Acetylamino-thiazole-5-sulfonyl chloride (CAS: 654072-71-6, 51 mg, 0.21 mmol) was added to a stirred solution of intermediate 8 (50 mg, 0.21 mmol, bis HCl salt) and diisopropylethylamine (0.15 mL, 0.85 mmol) in DCM (1.9 mL) at rt and the mixture was further stirred at rt for 3 h. NaHCO₃ (aq. sat. soltn.) was added and the mixture was further stirred at rt for 16 h. The solid was filtered off, washed with water and EtOAc/acetonitrile affording product 4 as a white solid (26 mg, 38% yield).

### E5. Preparation of Reference Product 5

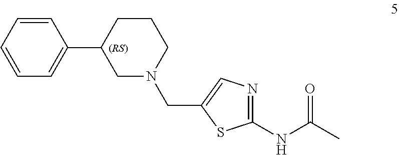 [SMILES: CC(=O)Nc1ncc(CN2CCCC(c3ccccc3)C2)s1]

3-Phenylpiperidine (CAS: 3973-62-4; 0.521 g, 3.23 mmol) was added at room temperature and under argon atmosphere to a solution of intermediate 12 (0.5 g, 2.95 mmol) in 1,2-dichloroethane (10 mL). Then acetic acid (0.1 mL), K-10 Montmorillonite (CAS: 1318-93-0; 0.5 g) and sodium triacetoxyborohydride (747 mg, 3.53 mmol) were added and the mixture was further stirred at 90° C. overnight. The reaction mixture was filtered through a Clarcel® bed and the filtrate was evaporated in vacuo. The residue thus obtained was purified by reverse phase column chromatography (C18, Acetonitrile/water (2/98 to 100/0), quenched with NaHCO₃ (aq. sat. soltn.). The desired fractions were concentrated in vacuo to yield product 5 as yellow solid (180 mg, 36% yield).

### E6. Preparation of Product 6

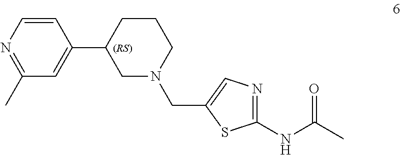 [SMILES: CC(=O)Nc1ncc(CN2CCCC(c3ccnc(C)c3)C2)s1]

Sodium triacetoxyborohydride (156.6 mg, 0.74 mmol) was added to a stirred solution of intermediate 11 (131.4 mg, 0.53 mmol, bis hydrochloric salt), intermediate 12 (179 mg, 1.05 mmol) and triethylamine (0.22 mL, 1.58 mmol) in dry THF (13 mL) at rt and under N₂ atmosphere. The mixture was further stirred at rt overnight. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by flash column chromatography (silica, MeOH in DCM, 0/100 to 10/100). The desired fractions were concentrated in vacuo to yield product 6 as a solid (34 mg, 19% yield).

### E7. Preparation of Product 7, 130 and 131

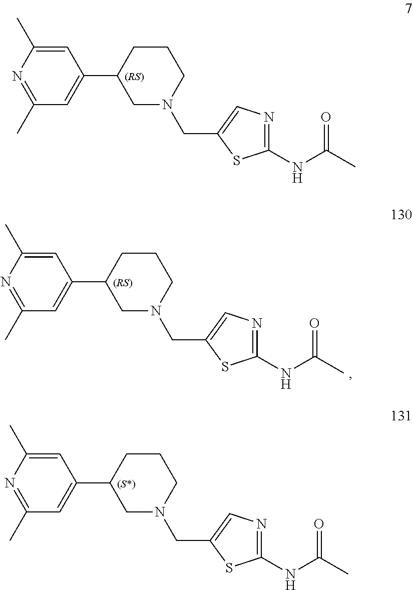 [SMILES: CC(=O)Nc1ncc(CN2CCCC(c3cc(C)nc(C)c3)C2)s1 ; CC(=O)Nc1ncc(CN2CCCC(c3cc(C)nc(C)c3)C2)s1 ; CC(=O)Nc1ncc(CN2CCCC(c3cc(C)nc(C)c3)C2)s1]

Sodium triacetoxyborohydride (241 mg, 1.14 mmol) was added to a stirred solution of intermediate 15 (214 mg, 0.81 mmol, bis hydrochloric salt), intermediate 12 (277 mg, 1.62 mmol) and triethylamine (0.34 mL, 2.44 mmol) in dry THF (20 mL) at rt and under N₂ atmosphere. The mixture was further stirred at rt overnight. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by flash column chromatography (silica, MeOH in DCM, 0/100 to 10/100). The desired fractions were concentrated in vacuo to yield product 7 as a solid (73 mg, 26% yield).

Product 7 (609 mg) was subjected to chiral SFC (stationary phase: chiralpak IG 5 μm 250*20 mm, mobile phase: 50% CO₂, 50% MeOH (0.3% iPrNH₂)) to yield product 130 (236 mg) and product 131 (246 mg) as pale yellow solids.

### E8. Preparation of Product 8

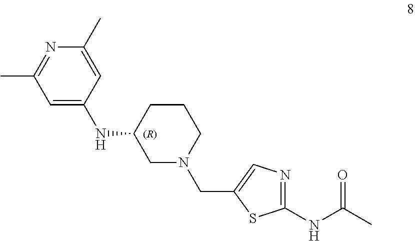

Acetic acid (0.023 mL, 0.4 mmol) was added to a stirred suspension of intermediate 17 (40 mg, 0.19 mmol), intermediate 12 (25 mg, 0.4 mmol) in MeOH (1 mL) at rt and under N₂ atmosphere. The mixture was further stirred at rt for 1 h and then sodium cyanoborohydride (25 mg, 0.4 mmol) was added. The mixture was further stirred at rt for 16 h. The reaction mixture was quenched with NaHCO₃ (aq. sat. soltn.) and diluted with DCM and then DCM/i-PrOH (9/1). The organic layer was separated, dried over MgSO₄, filtered and the filtrate was evaporated in vacuo. The residue thus obtained was purified by flash column chromatography (silica, 7N solution of NH₃ in MeOH in DCM, 0/100 to 10/90). The desired fractions were concentrated in vacuo to yield product 8 as a yellow solid (26.9 mg, 38% yield).

### E9. Preparation of Product 9

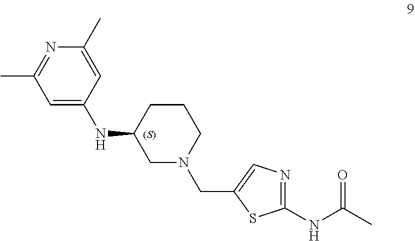

Acetic acid (0.020 mL, 0.34 mmol) was added to a stirred suspension of intermediate 19 (34 mg, 0.17 mmol), intermediate 12 (28 mg, 0.41 mmol) in MeOH (1 mL) at rt and under N₂ atmosphere. The mixture was further stirred at rt for 1 h and then sodium cyanoborohydride (28 mg, 0.44 mmol) was added. The mixture was further stirred at rt for 60 h. The reaction mixture was quenched with NaHCO₃ (aq. sat. soltn.) and extracted with DCM/i-PrOH (9/1). The organic layer was separated, dried over MgSO₄, filtered and the filtrate was evaporated in vacuo. The residue thus obtained was purified by reverse phase HPLC (Stationary phase: C18 XBridge® 30×100 mm 5 μm, mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 9 as a pale yellow solid (25.3 mg, 42% yield).

### E10. Preparation of Product 10

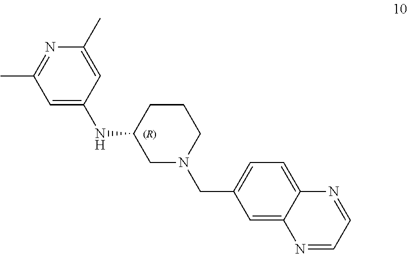

Acetic acid (0.023 mL, 0.4 mmol) was added to a stirred suspension of intermediate 17 (40 mg, 0.19 mmol), quinoxaline-6-carbaldehyde (CAS: 130345-50-5; 40 mg, 0.25 mmol) in MeOH (1 mL) at rt and under N₂ atmosphere. The mixture was further stirred at rt for 1 h and then sodium cyanoborohydride (25 mg, 0.4 mmol) was added. The mixture was further stirred at rt for 16 h. The reaction mixture was quenched with Na₂CO₃ (aq. sat. soltn.) and diluted with DCM. The organic layer was separated, dried over MgSO₄, filtered and the filtrate was evaporated in vacuo. The residue thus obtained was purified by flash column chromatography (SiO₂ amino functionalized, EtOAc in heptane, 0/100 to 100/0). The desired fractions were concentrated in vacuo to yield product 10 as yellow oil (11 mg, 16% yield).

### E11. Preparation of Product 11

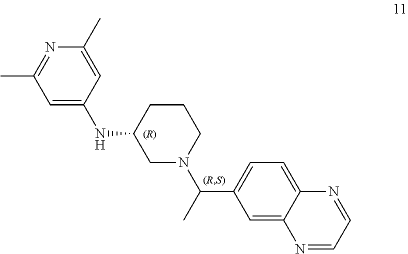

Titanium tetraisopropoxide (0.062 mL, 0.21 mmol) was added to a stirred solution of intermediate 17 (40 mg, 0.19 mmol), 1-(6-quinoxalinyl)ethanone (CAS: 83570-42-7; 45 mg, 0.26 mmol) in MeOH (1 mL) at rt and under N₂ atmosphere. The mixture was stirred at 80° C. for 16 h. Then sodium cyanoborohydride (20 mg, 0.32 mmol) was added and the mixture was stirred at 80° C. for 5 h and then at rt for 60 h. The volatiles were evaporated in vacuo. The residue thus obtained was purified by flash column chromatography (silica, 7N solution of NH₃ in MeOH in DCM, 0/100 to 10/90). The desired fractions were concentrated in vacuo to yield a fraction that was further purified by reverse phase HPLC (Stationary phase: C18 XBridge® 30×100 mm 5 μm, mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and extracted with EtOAc and DCM/2-PrOH (9/1). The desired fractions were collected and concentrated in vacuo to yield product 11 as yellow oil (7.7 mg, 11% yield).

### E12. Preparation of Product 12

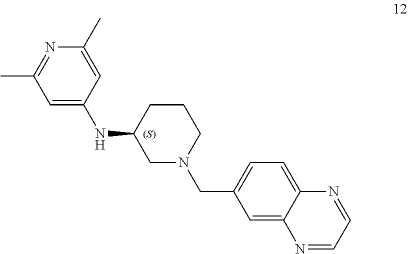

Acetic acid (0.020 mL, 0.35 mmol) was added to a stirred suspension of intermediate 19 (34 mg, 0.17 mmol), quinoxaline-6-carbaldehyde (CAS: 130345-50-5; 37 mg, 0.23 mmol) in MeOH (1 mL) at rt and under N₂ atmosphere. The mixture was further stirred at rt for 2.5 h and then sodium cyanoborohydride (34 mg, 0.54 mmol) was added. The mixture was further stirred at rt for 60 h. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by reverse phase HPLC (Stationary phase: C₁₈ XBridge® 30×100 mm 5 μm, mobile phase: gradient from 81% 10 mM NH₄CO₃H pH 9 solution in water, 19% CH₃CN to 64% 10 mM NH₄CO₃H pH 9 solution in water, 36% CH₃CN). The desired fractions were collected and concentrated in vacuo to yield product 12 as yellow oil (12.4 mg, 22% yield).

### E13. Preparation of Reference Product 13

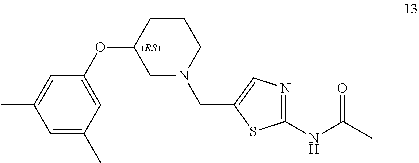 [SMILES: CC(=O)Nc1ncc(CN2CCCC(Oc3cc(C)cc(C)c3)C2)s1]

Sodium triacetoxyborohydride (63 mg, 0.3 mmol) was added to a stirred solution of crude intermediate 21 (77 mg), intermediate 12 (50 mg, 0.3 mmol) and triethylamine (0.1 mL, 0.72 mmol) in DCM (1.5 mL) at rt and under N₂ atmosphere. The mixture was further stirred at rt for 3 days. The reaction mixture was quenched with NaHCO₃ (aq. sat. soltn.). The organic layer was separated, dried over MgSO₄, filtered and the filtrate was evaporated in vacuo. The residue thus obtained was purified by flash column chromatography (silica, EtOAc in heptane, 0/100 to 80/20). The desired fractions were concentrated in vacuo to yield a residue that was further purified by reverse phase HPLC (Stationary phase: C18 XBridge® 30×150 mm, 5 μm, mobile phase: gradient from 81% 10 mM NH₄CO₃H pH 9 solution in water, 19% CH₃CN to 64% 10 mM NH₄CO₃H pH 9 solution in water, 36% CH₃CN), affording product 13 as a yellow film (6 mg, 7% yield).

### E14. Preparation of Product 14

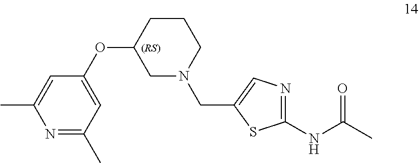 [SMILES: CC(=O)Nc1ncc(CN2CCCC(Oc3cc(C)nc(C)c3)C2)s1]

Sodium triacetoxyborohydride (42 mg, 0.2 mmol) was added to a stirred solution of crude intermediate 2 (35 mg, 0.125 mmol, bis-HCl salt), intermediate 12 (36 mg, 0.21 mmol) and triethylamine (0.07 mL, 0.5 mmol) in DCM (1 mL) at rt and under N₂ atmosphere. The mixture was further stirred at rt for 17 h. The reaction mixture was quenched with NaHCO₃ (aq. sat. soltn.). The organic layer was separated, dried over MgSO₄, filtered and the filtrate was evaporated in vacuo. The residue thus obtained was purified by reverse phase HPLC (Stationary phase: C₁₈ XBridge® 30×100 mm 5 μm, mobile phase: gradient from 60% 10 mM NH₄CO₃H pH 9 solution in water, 40% MeOH to 37% 10 mM NH₄CO₃H pH 9 solution in water, 63% MeOH), affording product 14 as yellow oil (12 mg, 27% yield).

### E15. Preparation of Product 15

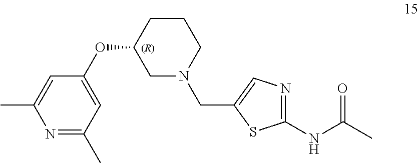

Diisopropylethylamine (0.46 mL, 2.66 mmol) was added to a stirred solution of intermediate 2a (110 mg, 0.53 mmol) in DCM (16 mL) at rt and the mixture was stirred at rt for 10 min. Intermediate 12 (109 mg, 0.64 mmol) was added and the mixture was stirred at rt for 2.5 h. Then, sodium triacetoxyborohydride (226 mg, 1.07 mmol) was added and the mixture was further stirred at rt for 68 h. The reaction mixture was quenched with water. The organic layer was separated, dried over Na₂SO₄, filtered and the filtrate was evaporated in vacuo. The residue thus obtained was purified by flash column chromatography (silica gel, MeOH in DCM, 0/100 to 15/85). The desired fractions were concentrated in vacuo to yield product 15 as pale yellow oil. This oil was taken up in Et₂O and HCl (0.44 mL, 6M solution in i-PrOH) was added. The mixture was stirred at rt for 10 min. The solvent was separated from the sticky solid formed. This solid was treated with EtOAc and the resulting suspension was filtered off. The solid was dried in the vacuum oven (50° C.) affording the HCl salt of product 15 as a pale yellow solid (69 mg, 31% yield).

### E16. Preparation of Product 16

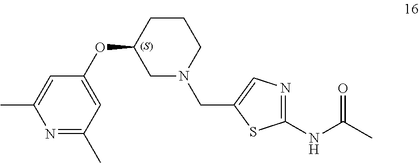

Diisopropylethylamine (0.24 mL, 1.4 mmol) was added to a stirred solution of intermediate 2b (78 mg, 0.28 mmol, bis HCl salt) in DCM (9 mL) at rt and the mixture was stirred at rt for 10 min. Intermediate 12 (57 mg, 0.33 mmol) was added and the mixture was stirred at rt for 2 h. Then, sodium triacetoxyborohydride (118 mg, 0.56 mmol) was added and the mixture was further stirred at rt for 64 h. The reaction mixture was quenched with water. The organic layer was separated, dried over Na₂SO₄, filtered and the filtrate was evaporated in vacuo. The residue thus obtained was purified by flash column chromatography (silica gel, MeOH in DCM, 0/100 to 15/85). The desired fractions were concentrated in vacuo to yield product 15 as a pale yellow oil. This oil was taken up in Et₂O and HCl (0.44 mL, 6M solution in i-PrOH) was added. The mixture was stirred at rt for 10 min. The solvent was separated from the sticky solid formed. This solid was treated with EtOAc and the resulting suspension was filtered off. The solid was dried in the vacuum oven (50° C.) affording the HCl salt of product 16 as pale yellow solid (58 mg, 48% yield).

### E17. Preparation of Product 17

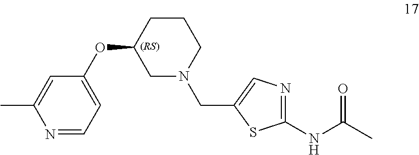

Diisopropylethylamine (0.94 mL, 0.54 mmol) was added to a stirred solution of intermediate 4 (29 mg, 0.11 mmol, bis HCl salt) in DCM (0.58 mL) at rt and the mixture was stirred at rt for 5 min. Intermediate 12 (22.3 mg, 0.13 mmol) and sodium triacetoxyborohydride (35 mg, 0.16 mmol) were added and the mixture was stirred at rt for 96 h. The reaction mixture was quenched with NaHCO₃ (aq. sat. soltn.). The organic layer was separated, dried over MgSO₄, filtered and the filtrate was evaporated in vacuo. The residue thus obtained was purified by flash column chromatography (silica gel, MeOH in DCM, 0/100 to 15/85). The desired fractions were concentrated in vacuo to yield product 17 as a transparent film (7.6 mg, 20% yield).

### E18. Preparation of Product 18

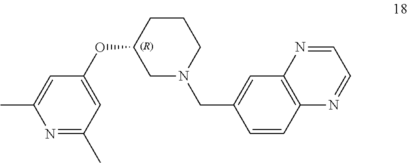

Diisopropylethylamine (0.177 mL, 1.03 mmol) was added to a stirred solution of intermediate 2a (50 mg, 0.21 mmol, HCl salt) in DCM (1.1 mL) at rt and the mixture was stirred at rt for 5 min, quinoxaline-6-carbaldehyde (CAS: 130345-50-5; 39 mg, 0.24 mmol) and sodium triacetoxyborohydride (65.5 mg, 0.31 mmol) were added and the mixture was stirred at rt for 16 h. The reaction mixture was quenched with NaHCO₃. The organic layer was separated, dried over MgSO₄, filtered and the filtrate was evaporated in vacuo. The residue thus obtained was purified by flash column chromatography (silica gel, MeOH in DCM, 0/100 to 10/90). The desired fractions were concentrated in vacuo to yield product 18 as a colorless sticky solid (33 mg, 46% yield).

### E19. Preparation of Product 19

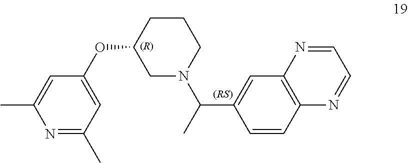

A mixture of triethylamine (0.034 mL, 0.25 mmol), intermediate 2a (30 mg, 0.12 mmol, HCl salt) and 6-(1-chloroethyl)-quinoxaline (CAS: 1884155-52-5; 40 mg, 0.12 mmol) in 1,2-dichloroethane (1.1 mL) at rt and the mixture was stirred at rt for 120 h. The volatiles were evaporated in vacuo. The residue thus obtained was purified by flash column chromatography (silica gel, MeOH in DCM, 0/100 to 10/90). The desired fractions were concentrated in vacuo to yield a residue that was further purified by reverse phase to yield HPLC (Stationary phase: C18 XBridge® 30×100 mm 5 μm, mobile phase: gradient from 81% 10 mM NH₄CO₃H pH 9 solution in water, 19% CH₃CN to 64% 10 mM NH₄CO₃H pH 9 solution in Water, 36% CH₃CN) product 19 (2.8 mg, 6% yield, mixture of diastereoisomers 55:45).

### E20. Preparation of Product 20

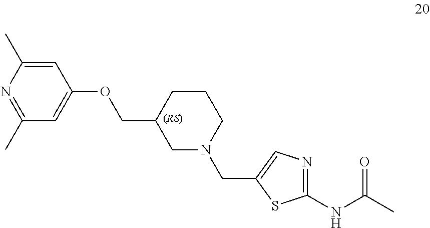 [SMILES: CC(=O)Nc1ncc(CN2CCCC(COc3cc(C)nc(C)c3)C2)s1]

Sodium triacetoxyborohydride (60.2 mg, 0.28 mmol) was added to a stirred solution of intermediate 23 (52 mg, bis hydrochloric salt), intermediate 12 (69.1 mg, 0.41 mmol) and triethylamine (0.085 mL, 0.61 mmol) in dry THF (5 mL) at rt and under N₂ atmosphere. The mixture was further stirred at rt overnight. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by flash column chromatography (silica, DCM:MeOH 10:1). The desired fractions were concentrated in vacuo to yield product 20 as a white solid (45 mg, 58% yield).

### E21. Preparation of Product 21

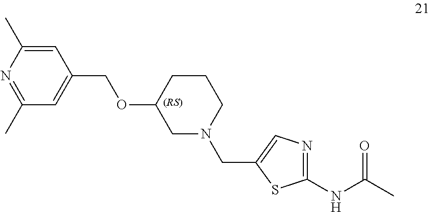 [SMILES: CC(=O)Nc1ncc(CN2CCCC(OCc3cc(C)nc(C)c3)C2)s1]

Sodium triacetoxyborohydride (130.8 mg, 0.61 mmol) was added to a stirred solution of intermediate 25 (75 mg, 0.343 mmol), intermediate 12 (70 mg, 0.41 mmol) in DCM (15 mL) at rt and under N₂ atmosphere. The mixture was further stirred at rt overnight. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by flash column chromatography (silica, MeOH in DCM, 0/100 to 1/10). The desired fractions were concentrated in vacuo to yield product 21 (67 mg, 46% yield) as colorless oil. This oil was taken up in DCM and 1 equivalent of HCl (4M solution in 1,4-dioxane) was added. The volatiles were evaporated in vacuo and the residue thus obtained was triturated with diisopropylether to yield the HCl salt of product 21 (56 mg, 42% yield).

### E22. Preparation of Product 22

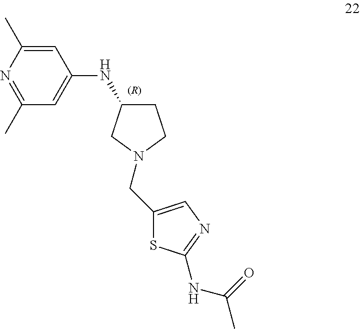

Sodium triacetoxyborohydride (166.2 mg, 0.78 mmol) and intermediate 12 (53.4 mg, 0.31 mmol) were added to a stirred solution of intermediate 27 (50 mg, 0.26 mmol) in DCM (3.5 mL) at rt. The mixture was further stirred at rt for 18 h. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by reverse phase HPLC (Stationary phase: C18 XBridge® 30×100 mm 5 μm, mobile phase: gradient from 80% 0.1% NH₄CO₃H/NH₄OH pH 9 solution in water, 20% CH₃CN to 0% 0.1% NH₄CO₃H/NH₄OH pH 9 solution in water, 100% CH₃CN). The desired fractions were concentrated in vacuo to yield a product fraction that further purified by flash column chromatography (silica; MeOH in DCM 0/100 to 10/90). The desired fractions were collected and concentrated in vacuo to yield product 22 as yellow solid (17 mg, 19% yield).

### E23. Preparation of Product 23

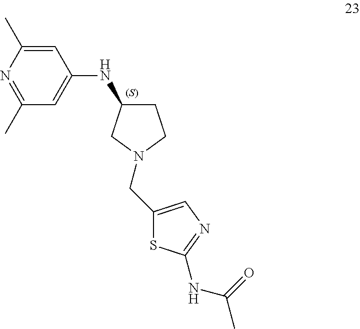

Sodium triacetoxyborohydride (166.2 mg, 0.78 mmol) and intermediate 12 (53.4 mg, 0.31 mmol) were added to a stirred solution of intermediate 27 (50 mg, 0.26 mmol) in DCM (3.5 mL) at rt. The mixture was further stirred at rt for 18 h. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by reverse phase HPLC (Stationary phase: C₁₈ XBridge® 30×100 mm 5 μm, mobile phase: gradient from 80% 0.1% NH₄CO₃H/NH₄OH pH 9 solution in water, 20% CH₃CN to 0% 0.1% NH₄CO₃H/NH₄OH pH 9 solution in water, 100% CH₃CN). The desired fractions were concentrated in vacuo to yield a product fraction that further purified by flash column chromatography (silica; MeOH in DCM 0/100 to 10/90). The desired fractions were collected and concentrated in vacuo to yield product 23 as yellow solid (19 mg, 21% yield).

### E24. Preparation of Product 24, 25 and 26

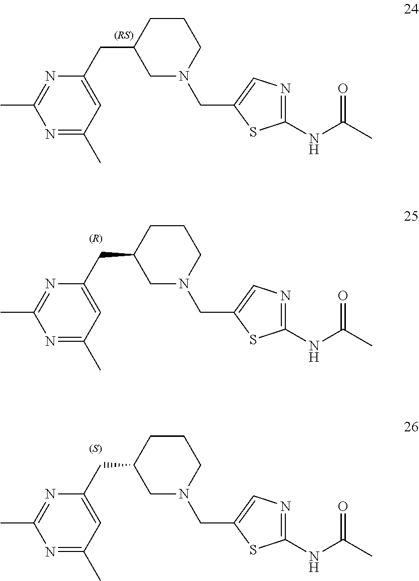

Intermediate 12 (1.16 g, 6.79 mmol) was added to a stirred solution of intermediate 32 (0.93 g, 4.53 mmol) in 1,2-dichloroethane (30.8 mL) at rt. The mixture was further stirred at rt for 30 min. Then, Sodium triacetoxyborohydride (1.92 g, 9 mmol) was added and then reaction mixture was stirred at room temperature overnight. The reaction mixture was quenched with NH₄OH (aq. sat. soltn.) and diluted with EtOAc. The organic layer was separated, dried over Na₂SO₄, filtered and the filtrate was evaporated in vacuo. The residue thus obtained was purified by automated flash chromatography (silica, 10% NH₃/MeOH in DCM, 0/100 to 10/90). The desired fractions were collected and concentrated in vacuo to yield product 24 as white foam (1.1 g, 68% yield).

Product 24 (1.1 g) was subjected to preparative SFC (Stationary phase: Chiralpak® Daicel IC 20×250 mm, Mobile phase: CO₂, iPrOH+0.4 iPrNH₂) to give product 25 (478 mg) and product 26 (449 mg) both as white foams.

### E25. Preparation of Product 27, 28 and 29

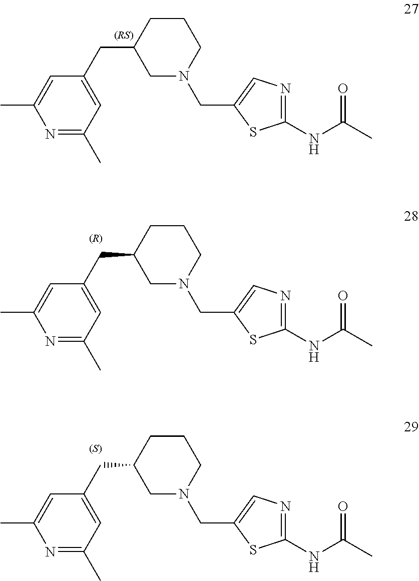

Intermediate 12 (1.17 g, 6.9 mmol) was added to a stirred solution of intermediate 34 (0.94 g, 4.6 mmol) in 1,2-dichloroethane (31.2 mL) at rt. The mixture was further stirred at rt for 30 min. Then, sodium triacetoxyborohydride (1.95 g, 9.2 mmol) was added and then reaction mixture was stirred at room temperature overnight. The reaction mixture was quenched with NH₄OH (aq. sat. soltn.) and diluted with EtOAc. The organic layer was separated, dried over Na₂SO₄, filtered and the filtrate was evaporated in vacuo. The residue thus obtained was purified by automated flash chromatography (silica, 10% NH₃/MeOH in DCM, 0/100 to 10/90). The desired fractions were collected and concentrated in vacuo to yield product 27 as yellow foam (1.2 g, 73% yield).

Product 27 (1.2 g) was subjected to preparative SFC (Stationary phase: Chiralpak® Daicel IC 20×250 mm, Mobile phase: CO₂, iPrOH+0.4 iPrNH₂) to give product 28 (565 mg) and product 29 (508 mg) both as white solids after crystallization with acetonitrile.

Alternatively, product 28 was prepared by the following reaction procedure: triethylamine (40.11 mL, 288.6 mmol) was added to a stirred slurry of intermediate (3R)-34 (20 g, 72.14 mmol) in acetonitrile (200 mL) at 10° C. under nitrogen (400 mL EasyMax vessel, overhead stirrer). Batch was warmed to 20° C. after addition and intermediate 12 (14.73 g, 86.5 mmol) was added. Reaction mixture was then stirred for 30 min and sodium triacetoxyborohydride (45.87 g, 216.4 mmol) was added portion-wise. Batch was stirred for 2 h and then warmed to 50° C. and stirred for 15 min at this temperature. The reaction mixture was cooled down to 20° C. and quenched with water (200 mL) and ammonium chloride (100 mL aq. sat. soltn.). EtOAc (200 mL) was then added and phases separated (aqueous pH 6 approx., desired product in the aqueous layer). Organic layer was then back-extracted with water (2×200 mL). EtOAc (300 mL) was then added to the combined aqueous layers and pH adjusted to 7 by addition of 2N NaOH. Phases were separated and aqueous back-extracted with EtOAc (2×200 mL). Combined organics were washed with brine (300 mL) and dried over MgSO₄. Solids were filtered and solvents distilled under reduced pressure to dryness. Crude material was purified by normal phase column chromatography (silica, MeOH in DCM 0/100 to 8/92). The desired fractions were collected and solvents were evaporated under reduced pressure to yield product 28 (213 g, 86% yield) as a light yellow colored solid.

### E26. Preparation of Product 30

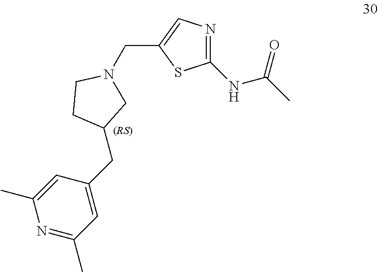 [SMILES: CC(=O)Nc1ncc(CN2CCC(Cc3cc(C)nc(C)c3)C2)s1]

Intermediate 12 (93 mg, 0.55 mmol) was added to a stirred solution of intermediate 37 (83 mg, 0.27 mmol, trifluoroacetate salt) in DCM (1.5 mL) at rt. The mixture was further stirred at rt for 30 min. Then, sodium triacetoxyborohydride (231.2 mg, 1.09 mmol) was added and then reaction mixture was stirred at room temperature overnight. Then additional sodium triacetoxyborohydride (115.5 mg, 0.5 mmol) was added and then reaction mixture was stirred at room temperature for 3 h. Then additional sodium triacetoxyborohydride (115.5 mg, 0.5 mmol) was added and then reaction mixture was stirred at room temperature for 2 h. The reaction mixture was quenched with NH₄OH (aq. sat. soltn.) and diluted with EtOAc. The organic layer was separated, dried over Na₂SO₄, filtered and the filtrate was evaporated in vacuo. The residue thus obtained was purified by automated flash chromatography (silica, EtOAc in heptane, 0/100 to 100/0 and then MeOH in EtOAc, 0/100 to 10/90). The desired fractions were collected and concentrated in vacuo to yield a fraction containing product that was further purified by reverse phase HPLC (Stationary phase: C18 XBridge® 30×100 mm 5 μm, mobile phase: gradient from 81% 0.1% NH₄CO₃H/NH₄OH pH 9 solution in water, 19% CH₃CN to 64% 0.1% NH₄CO₃H/NH₄OH pH 9 solution in water, 36% CH₃CN), to yield product 30 as a white solid (17.1 mg, 18.2% yield).

### E27. Preparation of Product 31

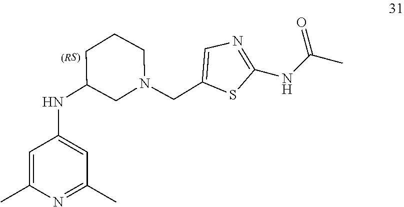 [SMILES: CC(=O)Nc1ncc(CN2CCCC(Nc3cc(C)nc(C)c3)C2)s1]

Triethylamine (0.26 mL, 1.86 mmol) was added to racemic intermediate 17 (150 mg, 0.62 mmol, HCl salt) in DCM/MeOH. The mixture was stirred for 10 min and then the volatiles were evaporated in vacuo. The residue thus obtained was taken up in dry THF (3 mL) and then intermediate 12 (211.2 mg, 1.24 mmol) and sodium triacetoxyborohydride (184.1 mg, 0.87 mmol) were added at rt. The mixture was further stirred at rt for 8 h. Then, acetic acid (0.035 mL, 0.62 mmol) and additional sodium triacetoxyborohydride (184.1 mg, 0.87 mmol) were added at rt and the mixture was stirred at rt overnight. Then, sodium triacetoxyborohydride (184.1 mg, 0.87 mmol) and additional intermediate 12 (52.8 mg, 0.31 mmol) were added and then reaction mixture was stirred at rt 18 h. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by reverse phase chromatography, 90% 25 mM NH₄CO₃H-10% CH₃CN/MeOH (1:1) to 54% 25 mM NH₄CO₃H-46% CH₃CN/MeOH (1:1), to yield product 31 (42.3 mg, 18.6% yield).

### E28. Preparation of Product 32

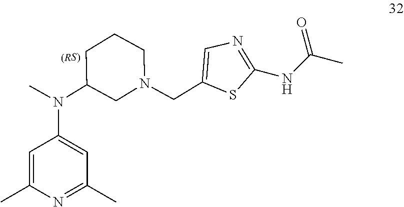 [SMILES: CC(=O)Nc1ncc(CN2CCCC(N(C)c3cc(C)nc(C)c3)C2)s1]

Sodium triacetoxyborohydride (72 mg, 0.25 mmol, bis HCl salt) and intermediate 12 (83.8 mg, 0.492 mmol) were added to intermediate 41 (54 mg, 0.246 mmol) in dry THF (7.5 mL) at rt under N₂ atmosphere. The mixture was further stirred at rt overnight. Then acetic acid (0.014 mL, 0.246 mmol) and additional intermediate 12 (20 mg, 0.118 mmol) were added at rt and the reaction mixture was further stirred under N₂ atmosphere overnight. The reaction mixture was quenched with NaHCO₃ (aq. sat. soltn.) and diluted with DCM. The organic layer was separated, dried over MgSO₄, filtered and the filtrate was evaporated in vacuo. The residue thus obtained was purified by reverse phase chromatography (started: organic phase 5%/aqueous phase 95%; finished: organic phase 37%/aqueous phase 63%. Organic phase: acetonitrile:MeOH 1:1; aqueous phase: 65 mM NH₄OAc:acetonitrile 90:10). The desired fractions were concentrated in vacuo to yield product 32 (12 mg, 13% yield).

### E29. Preparation of Product 33

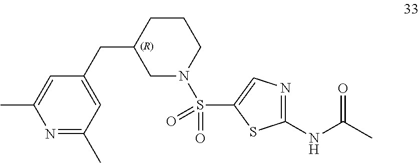

2-Acetylamino-thiazole-5-sulfonyl chloride (CAS: 654072-71-6, 140 mg, 0.58 mmol) was added portion wise to a stirred solution of intermediate (3R)—I-30 (118.8 mg, 0.58 mmol, bis HCl salt) and diisopropylethylamine (0.32 mL, 1.86 mmol) in DCM (1.62 mL) at 0° C. and the mixture was further stirred at 0° C. for 1 h. NaHCO₃ (aq. sat. soltn.) was added and the organic layer was separated dried over MgSO₄, filtered and evaporated under vacuum.

The residue thus obtained was purified by automated flash chromatography (silica, 7N solution of NH₃ in MeOH in DCM, 0/100 to 4/96). The desired fractions were collected and concentrated in vacuo to yield product 33 as a white solid (53.8 mg, 23% yield).

### Preparation of Products 34-43, 45-77, 79-86, 89-92, 97-99, 101-113, 115, 126-129, 132, 140, 143, 145-147, 150-155, 157-166 and 169

The following compounds were prepared following a reductive amination procedure like the one described for the preparation of product 20 starting from the corresponding amine and aldehyde intermediates using sodium triacetoxyborohydride in DCM. Changes of solvent, reductant are mentioned in the Table below. In the case a base or acid was used this is also noted in the Table A below.

| | | INTERMEDIATE | |
|---|---|---|---|
| PRODUCT | INTERMEDIATE AMINE | ALDEHYDE | COMMENT |
| 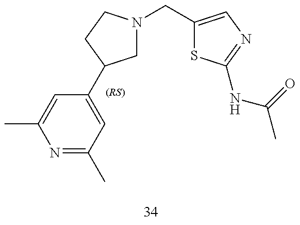 [SMILES: CC(=O)Nc1ncc(CN2CCC(c3cc(C)nc(C)c3)C2)s1] | 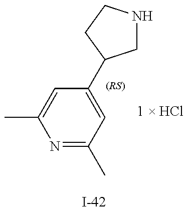 | I-12 | Solvent: 1,2- dichloroethane |
| 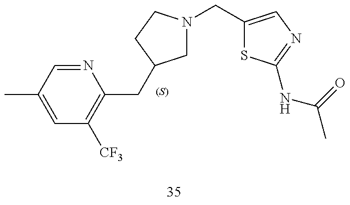 | 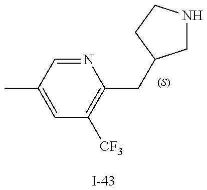 | I-12 | — |
| 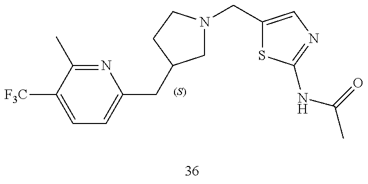 | 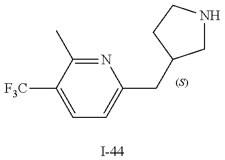 | I-12 | — |
| 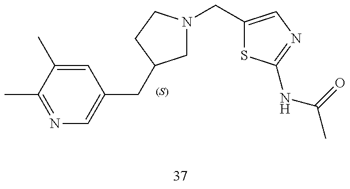 | 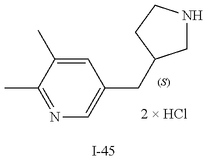 | I-12 | Base: NEt₃ |
| 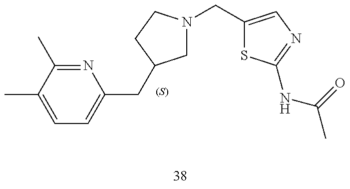 | 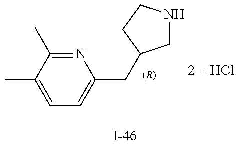 | I-12 | Base: NEt₃ |
| 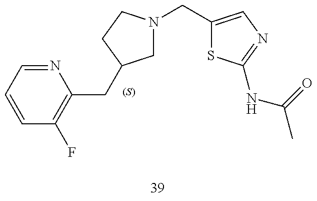 | 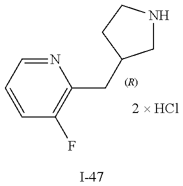 | I-12 | Base: NEt₃ |
| 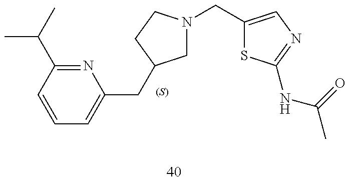 | 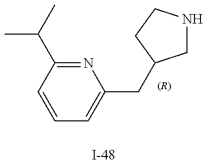 | I-12 | Solvent: 1,2- dichloro- ethane |
| 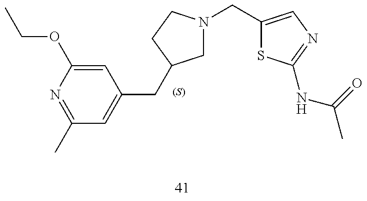 | 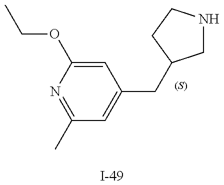 | I-12 | — |
| 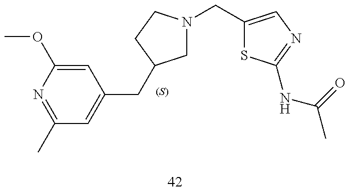 | 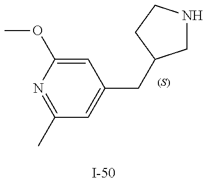 | I-12 | — |
| 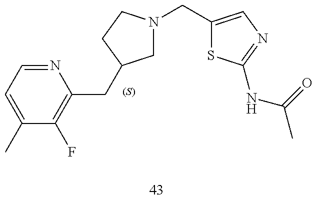 | 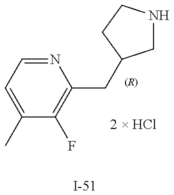 | I-12 | Base: NEt₃ |
| 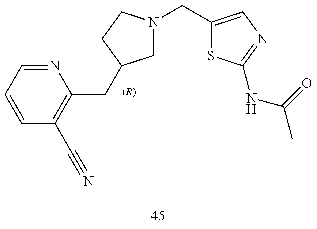 | 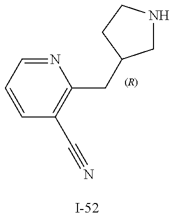 | I-12 | — |
| 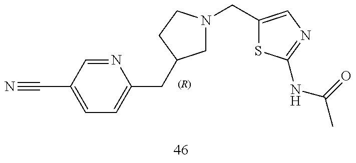 | 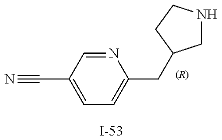 | I-12 | — |
| 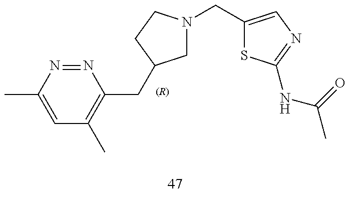 | 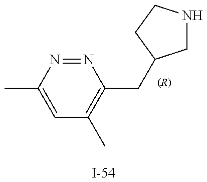 | I-12 | — |
| 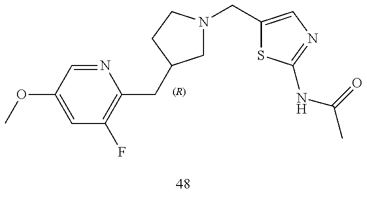 | 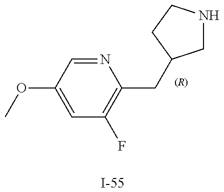 | I-12 | — |
| 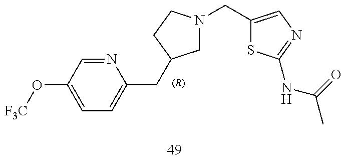 | 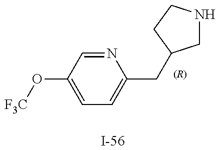 | I-12 | Solvent: 1,2- dichloro- ethane |
| 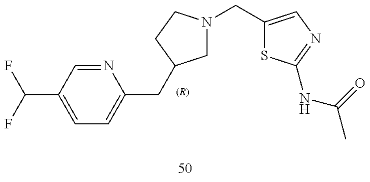 | 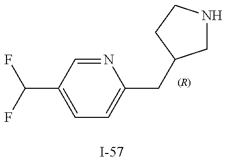 | I-12 | Solvent: 1,2- dichloro- ethane |
| 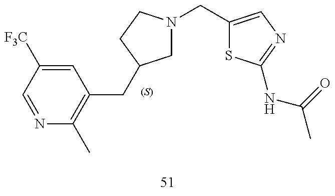 | 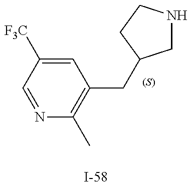 | I-12 | — |
| 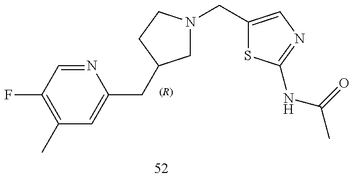 | 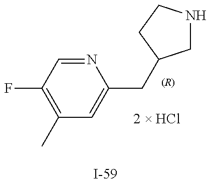 | I-12 | Base: NEt₃ |
| 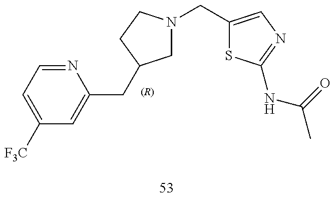 | 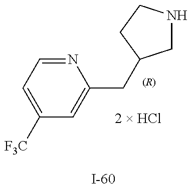 | I-12 | Base: NEt₃ |
| 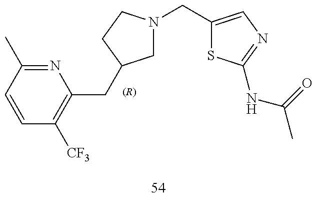 | 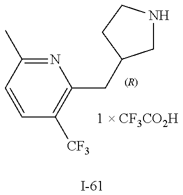 | I-12 | — |
| 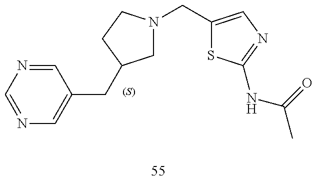 | 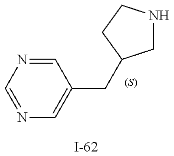 | I-12 | — |
| 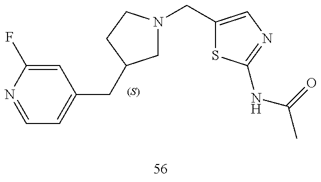 | 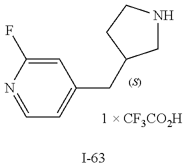 | I-12 | Base: NEt₃ additive: catalytic CH₃COOH |
| 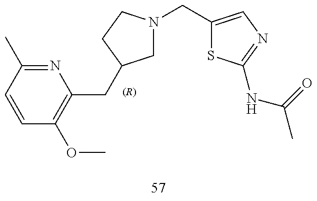 | 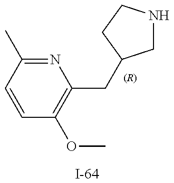 | I-12 | — |
| 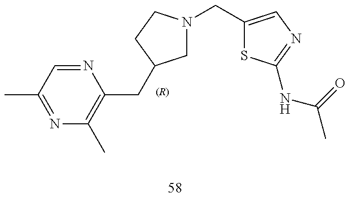 | 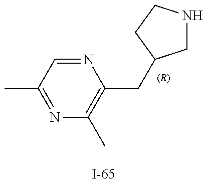 | I-12 | — |
| 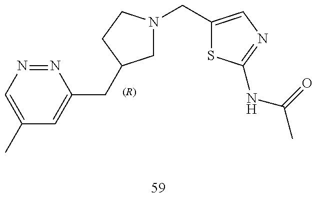 | 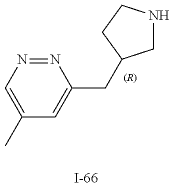 | I-12 | — |
| 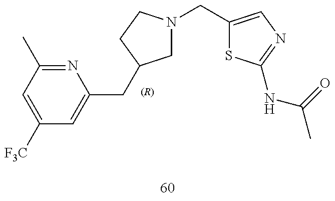 | 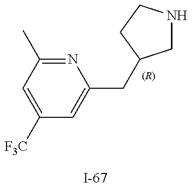 | I-12 | — |
| 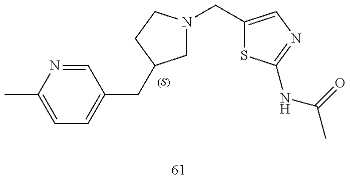 | 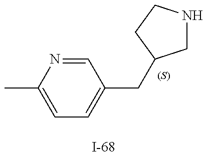 | I-12 | — |
| 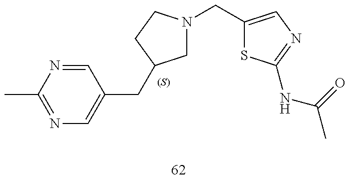 | 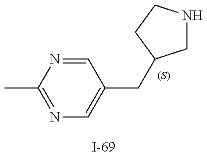 | I-12 | Solvent: 1,2- dichloro- ethane |
| 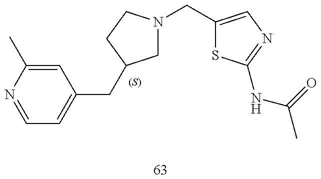 | 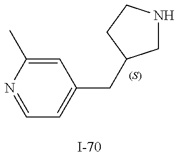 | I-12 | — |
| 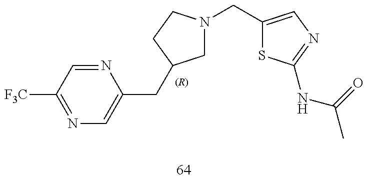 | 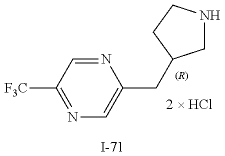 | I-12 | Base: NEt₃ |
| 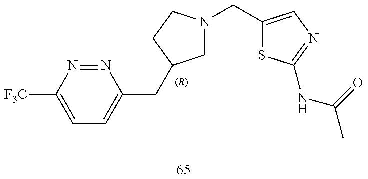 | 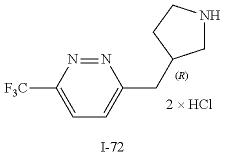 | I-12 | Base: NEt₃ |
| 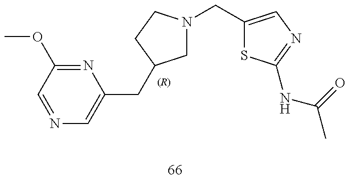 | 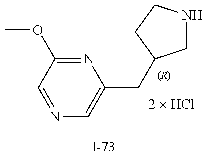 | I-12 | Base: NEt₃ |
| 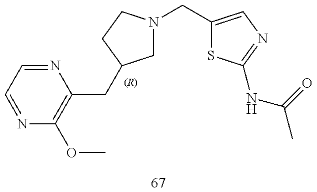 | 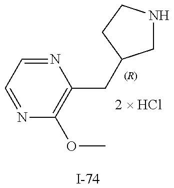 | I-12 | Base: NEt₃ |
| 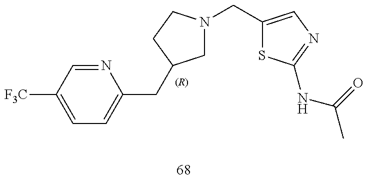 | 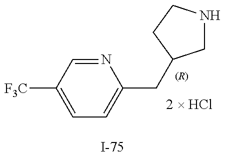 | I-12 | Base: NEt₃ |
| 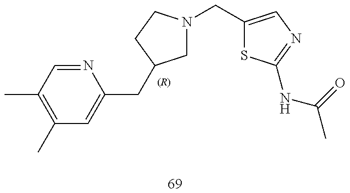 | 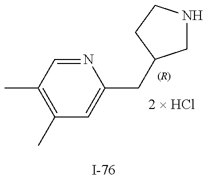 | I-12 | Base: NEt₃ |
| 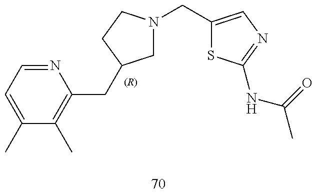 | 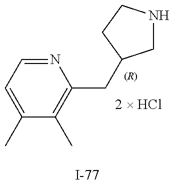 | I-12 | Base: NEt₃ |
| 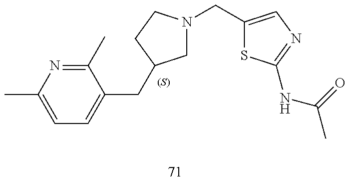 | 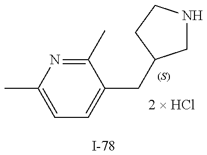 | I-12 | Base: NEt₃ |
| 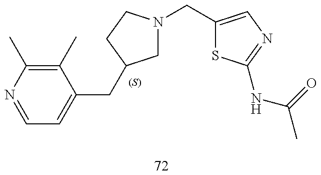 | 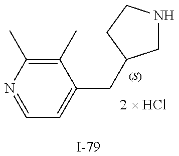 | I-12 | Base: NEt₃ |
| 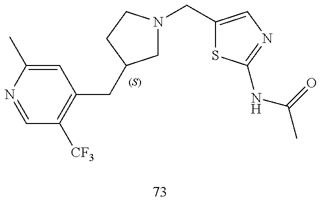 | 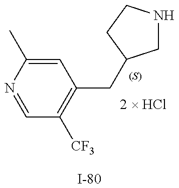 | I-12 | Base: NEt₃ |
| 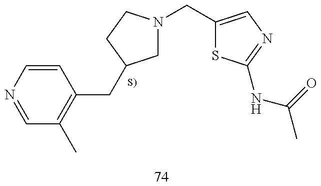 | 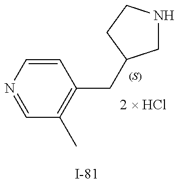 | I-12 | Base: NEt₃ |
| 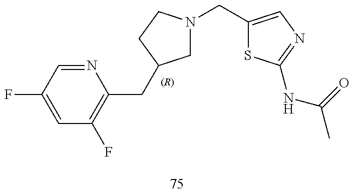 | 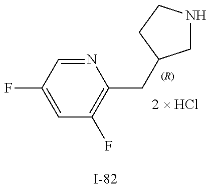 | I-12 | Base: NEt₃ |
| 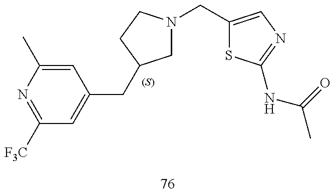 | 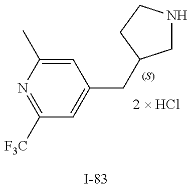 | I-12 | Base: NEt₃ |
| 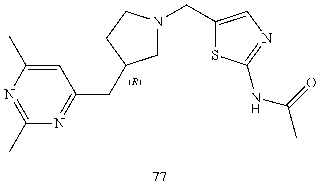 | 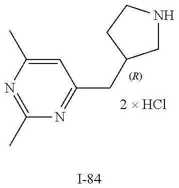 | I-12 | Base: NEt₃ |
| 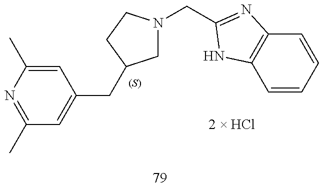 | 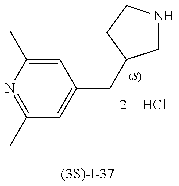 | 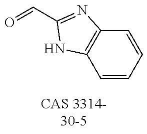 [SMILES: O=Cc1nc2ccccc2[nH]1] | Base: NEt₃ co-solvent: MeOH |
| 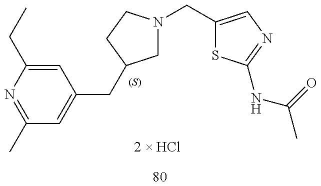 | 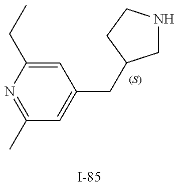 | I-12 | — |
| 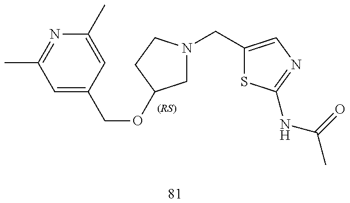 | 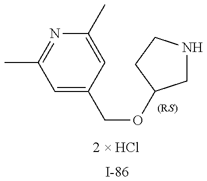 | I-12 | — |
| 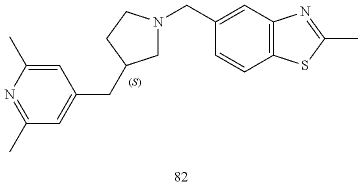 | 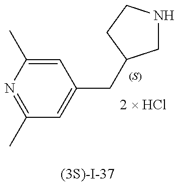 | 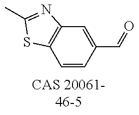 | Base: NEt₃ co-solvent: MeOH |
| 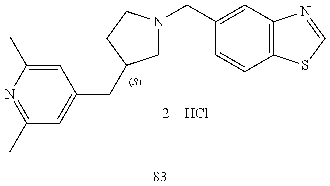 | 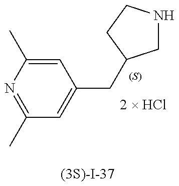 | 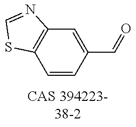 [SMILES: O=Cc1ccc2scnc2c1] | Base: NEt₃ co-solvent: MeOH |
| 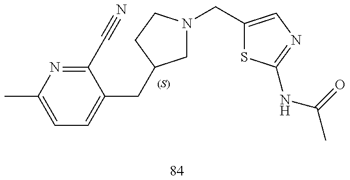 | 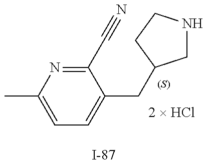 | I-12 | Base: NEt₃ |
| 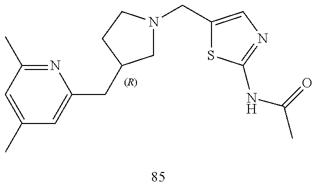 | 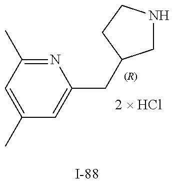 | I-12 | Base: NEt₃ |
| 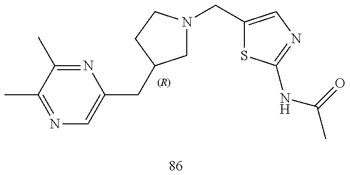 | 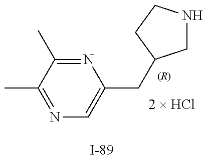 | I-12 | Base: NEt₃ |
| 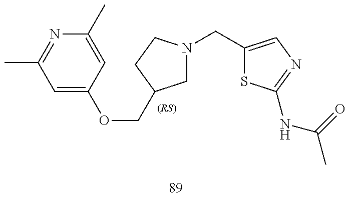 | 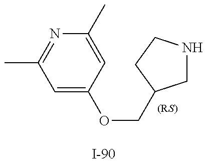 | I-12 | co-solvent: MeOH |
| 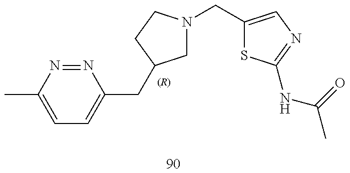 | 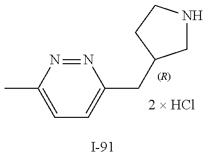 | I-12 | Base: NEt₃ |
| 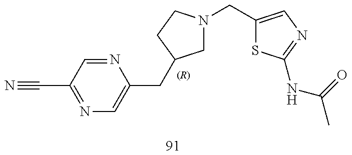 | 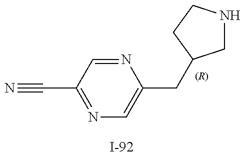 | I-12 | Base: NEt₃ |
| 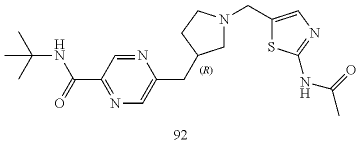 | 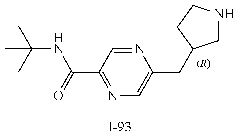 | I-12 | Base: NEt₃ |
| 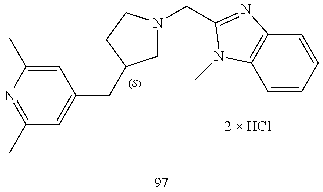 | 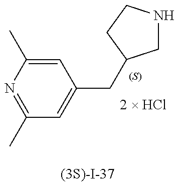 | CAS 3012- 80-4 | Base: NEt₃ co-solvent: MeOH |
| 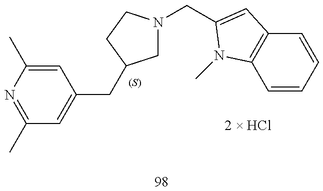 | 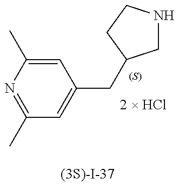 | CAS 27421- 51-8 | Base: NEt₃ |
| 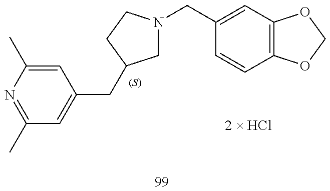 | 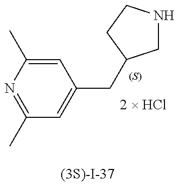 | CAS 120-57- 0 | Base: NEt₃ |
| 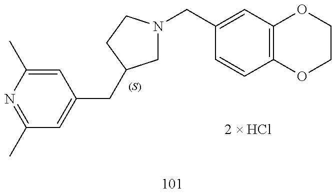 | 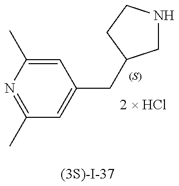 | CAS 29668- 44-8 | Base: NEt₃ |
| 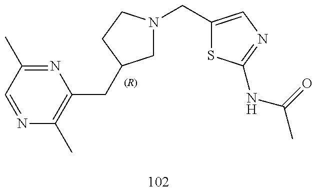 | 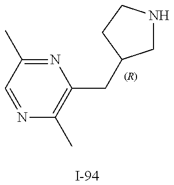 | I-12 | Base: NEt₃ |
| 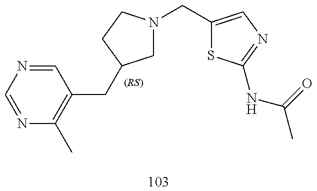 | 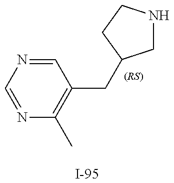 | I-12 | — |
| 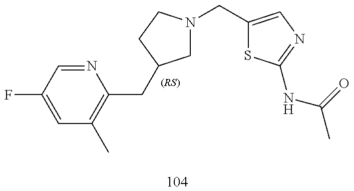 | 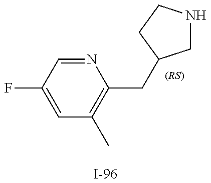 | I-12 | Base: NEt₃ |
| 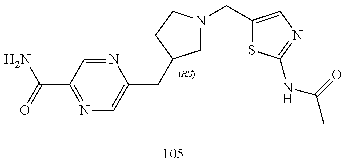 [SMILES: CC(=O)Nc1ncc(CN2CCC(Cc3cnc(C(N)=O)cn3)C2)s1 ; "105"] | 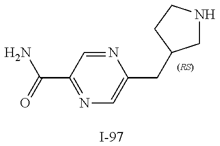 | I-12 | — |
| 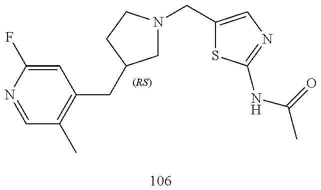 [SMILES: CC(=O)Nc1ncc(CN2CCC(Cc3cc(F)ncc3C)C2)s1 ; "106"] | 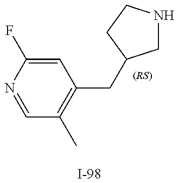 | I-12 | — |
| 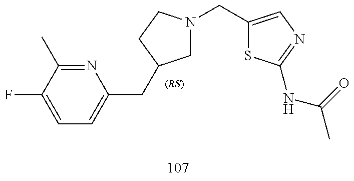 [SMILES: CC(=O)Nc1ncc(CN2CCC(Cc3ccc(F)c(C)n3)C2)s1] | 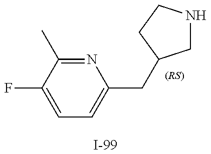 | I-12 | — |
| 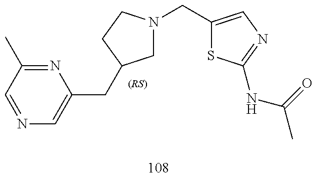 [SMILES: CC(=O)Nc1ncc(CN2CCC(Cc3cncc(C)n3)C2)s1] | 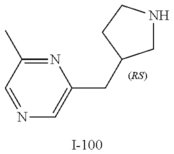 | I-12 | — |
| 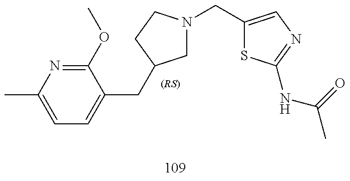 [SMILES: COc1nc(C)ccc1CC1CCN(Cc2cnc(NC(C)=O)s2)C1] | 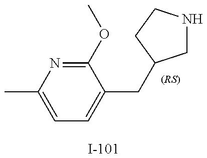 | I-12 | — |
| 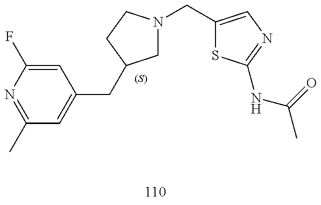 | 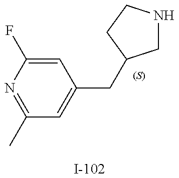 | I-12 | — |
| 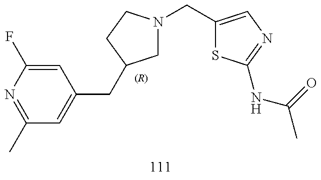 | 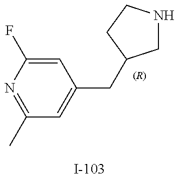 | I-12 | — |
| 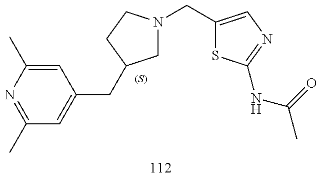 | 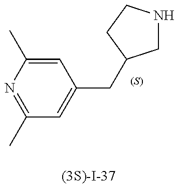 | I-12 | — |
| 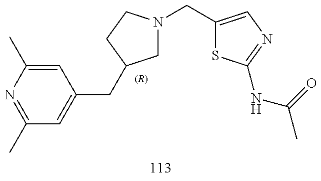 | 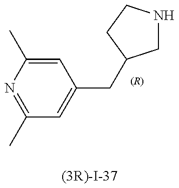 | I-12 | — |
| 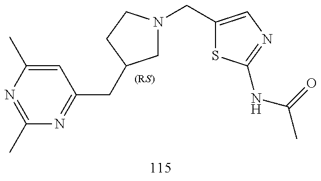 | 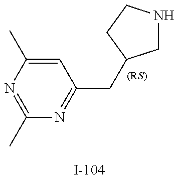 | I-12 | — |
| 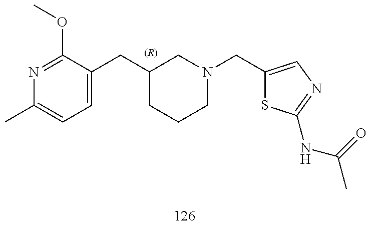 | 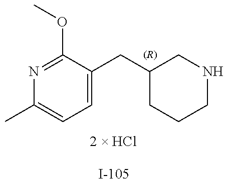 | I-12 | Base: NEt₃ |
| 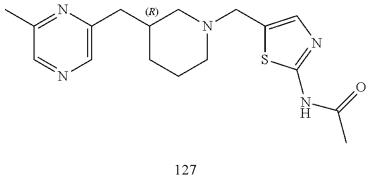 | 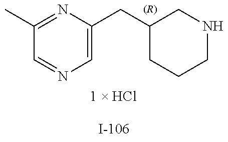 | I-12 | Base: NEt₃ |
| 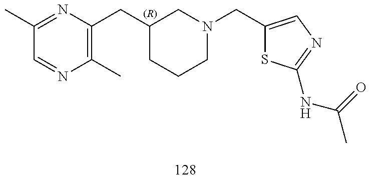 | 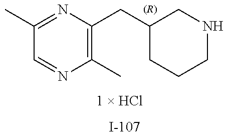 | I-12 | Base: NEt₃ |
| 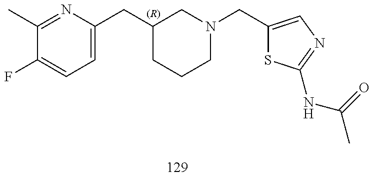 | 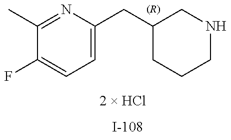 | I-12 | Base: NEt₃ |
| 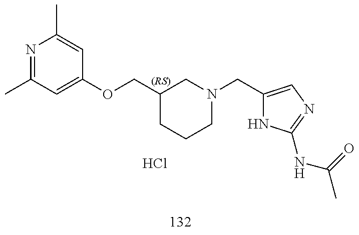 | 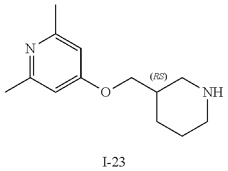 | CAS: 917919-66-5 | Reductant: sodium cyano- borohydride Solvent: MeOH Acid: CH3COOH |
| 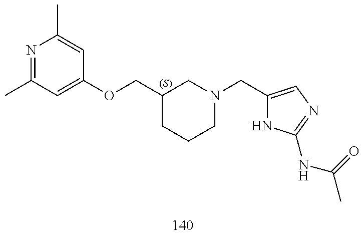 | 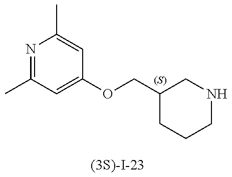 | I-12 | Reductant: sodium cyano- borohydride |
| 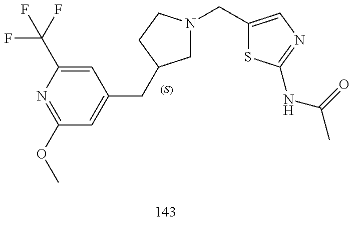 | 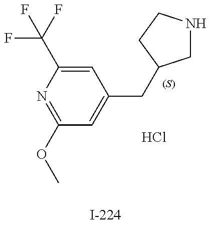 | I-12 | Solvent: 1,2- dichloro- ethane |
| 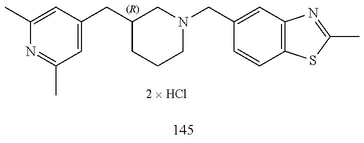 | 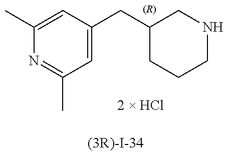 | CAS 20061- 46-5 | Base: NEt₃ |
| 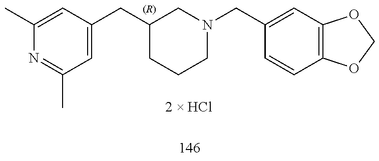 | 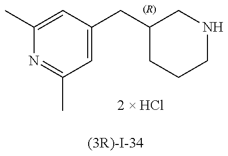 | CAS 120-57- 0 | Base: NEt₃ |
| 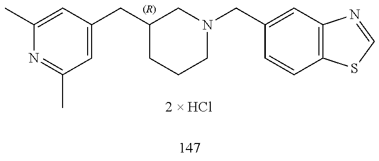 | 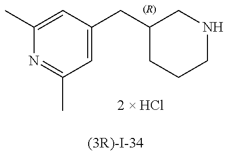 | CAS 211915- 06-9 | Base: NEt₃ co-solvent: MeOH |
| 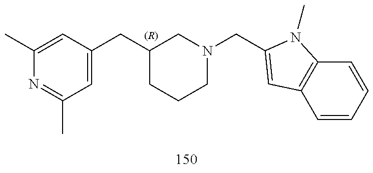 | 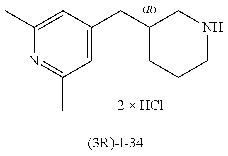 | CAS 27421- 51-8 | Base: NEt₃ co-solvent: MeOH |
| 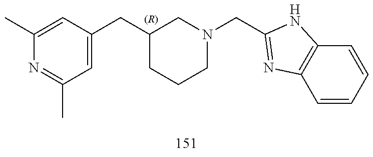 | 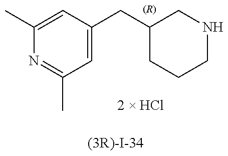 | CAS 3314- 30-5 | Base: NEt₃ co-solvent: MeOH |
| 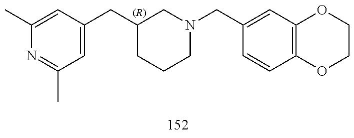 | 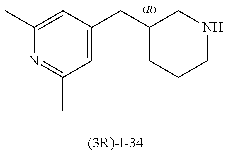 | CAS 29668- 44-8 | — |
| 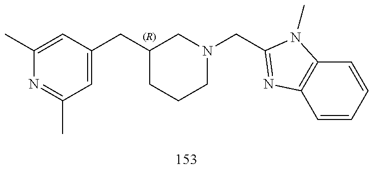 | 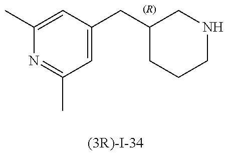 | CAS 3012- 80-4 | co-solvent: MeOH |
| 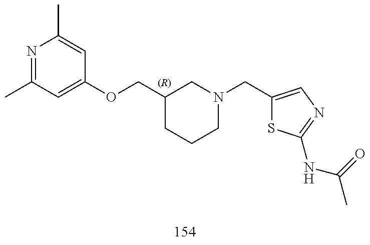 | 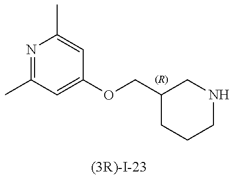 | I-12 | — |
| 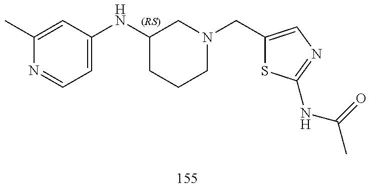 | 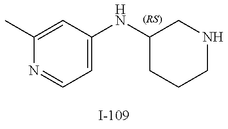 | I-12 | Solvent: THF Additive: CH₃COOH |
| 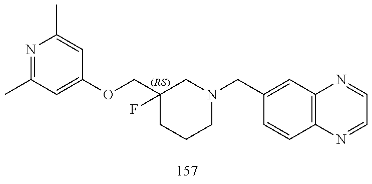 | 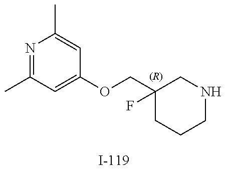 | CAS 130345- 50-5 | Reductant: sodium cyano- borohydride Solvent: MeOH Additive: CH₃COOH |
| 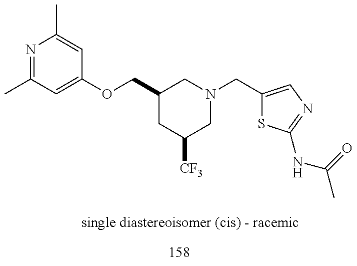 | 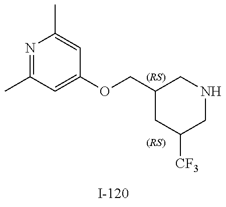 | I-12 | Reductant: sodium cyano- borohydride Solvent: MeOH Additive: CH₃COOH |
| 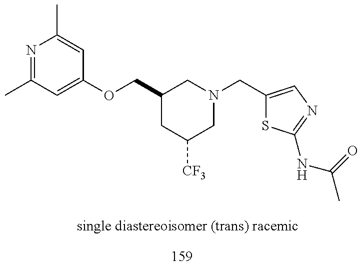 | 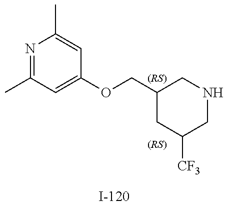 | I-12 | Reductant: sodium cyano- borohydride Solvent: MeOH Additive: CH₃COOH |
| 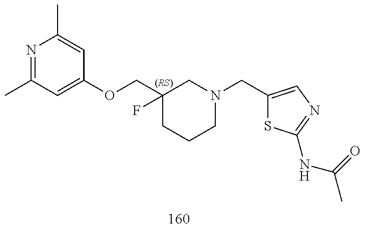 [SMILES: CC(=O)Nc1ncc(CN2CCCC(F)(COc3cc(C)nc(C)c3)C2)s1] | 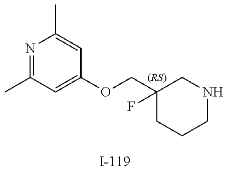 | I-12 | Reductant: sodium cyano- borohydride Solvent: MeOH Additive: CH₃COOH/ CH₃COONa |
| 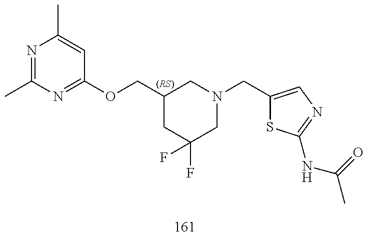 [SMILES: CC(=O)Nc1ncc(CN2CC(COc3cc(C)nc(C)n3)CC(F)(F)C2)s1] | 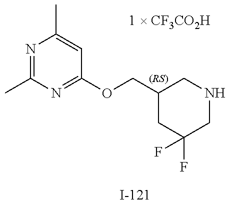 | I-12 | Reductant: sodium cyano- borohydride Solvent: MeOH Additive: CH₃COOH/ CH₃COONa |
| 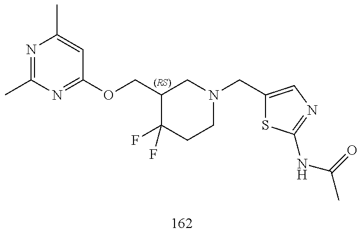 [SMILES: CC(=O)Nc1ncc(CN2CCC(F)(F)C(COc3cc(C)nc(C)n3)C2)s1] | 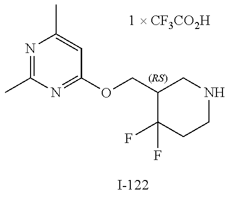 | I-12 | Reductant: sodium cyano- borohydride Solvent: MeOH Additive: CH₃COOH/ CH₃COONa |
| 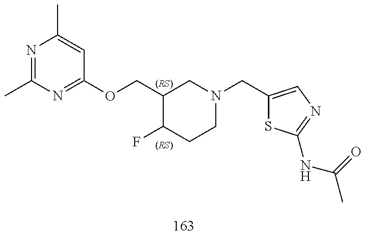 [SMILES: CC(=O)Nc1ncc(CN2CCC(F)C(COc3cc(C)nc(C)n3)C2)s1] | 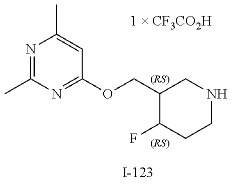 | I-12 | Reductant: sodium cyano- borohydride Solvent: MeOH Additive: CH₃COOH/ CH₃COONa |
| 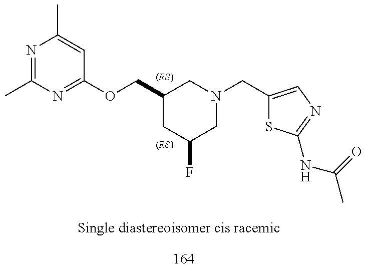 | 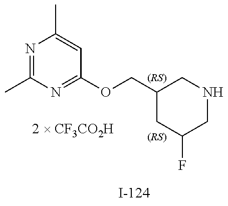 | I-12 | Reductant: sodium cyano- borohydride Solvent: MeOH Additive: CH₃COOH/ CH₃COONa |
| 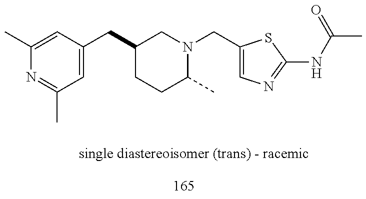 | 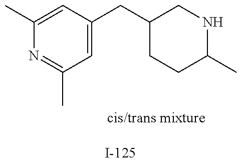 | I-12 | Base: NEt₃ solvent: ACN |
| 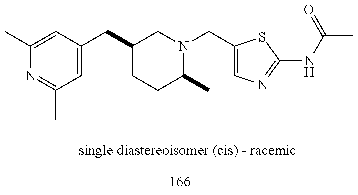 | 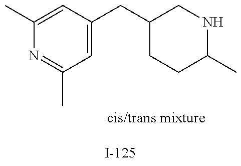 | I-12 | Base: NEt₃ solvent: ACN |
| 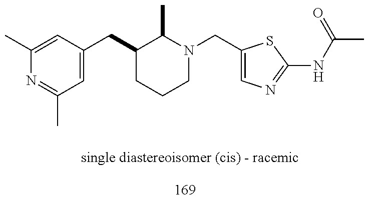 | 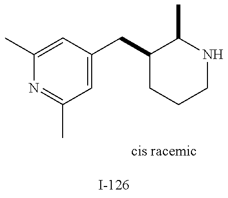 | I-12 | Reductant: NaBH(OAc)₃/ NaBH₃CN solvent: DCM/MeOH additive: CH₃COOH |

### E30. Preparation of Product 44

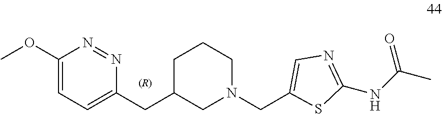

Sodium methoxide (0.3 mL, 1.63 mmol, 30% in MeOH) was added to a stirred solution on intermediate 111 (20 mg, 0.048 mmol) and CuI (11 mg, 0.058 mmol) in DMF (0.3 mL) under N₂ atmosphere. The tube was sealed and the mixture stirred at 100° C. for 1 h. Then the reaction mixture was diluted with EtOAc and sequentially washed with NH₄OH (aq, sat. sltn.) and brine. The organic layer was dried (Na₂SO₄), filtered and concentrated in vacuo. The crude was purified by ion exchange chromatography using an ISOLUTE® SCX2 cartridge eluting with 7M solution of ammonia in methanol. The desired fractions were collected and concentrated in vacuo. The resultant oil was purified by RP HPLC (Stationary phase: C18 XBridge® 30×100 mm 5 μm), Mobile phase: Gradient from 80% NH₄HCO₃ 0.25% solution in water, 20% CH₃CN to 60% NH₄HCO₃ 0.25% solution in water, 40% CH₃CN) The desired fractions were concentrated in vacuo to yield product 44 (6 mg, 36% yield) as white solid.

### Preparation of Products 78, 87, 88, 93, 94, 95, 96, 100, 114, 116-119, 139, 141, 142, 144, 148, 149, 156

The following compounds were prepared following a reductive amination procedure like the one described for the preparation of product 11 starting from the corresponding amine and methylketone intermediates using triethyl amine, sodium cyanoborohydride and titanium tetraisopropoxide in DCM. Changes of solvent, reductant are mentioned in Table B below.

| | | INTERMEDIATE | |
|---|---|---|---|
| PRODUCT | INTERMEDIATE AMINE | ALDEHYDE | COMMENT |
| 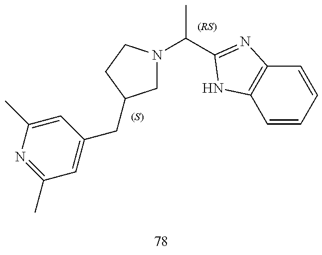 | 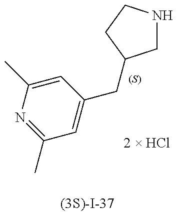 | CAS 18773- 95-0 | — |
| 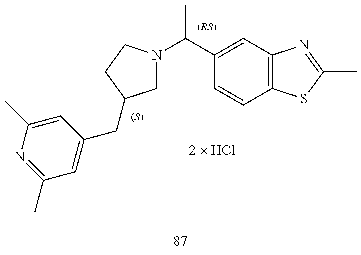 | 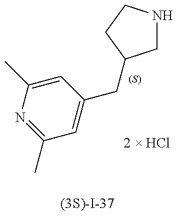 | CAS 20077- 88-7 | — |
| 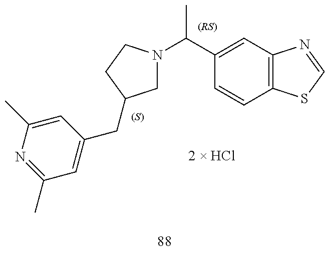 | 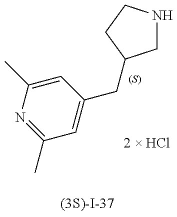 | CAS 90347- 90-3 | — |
| 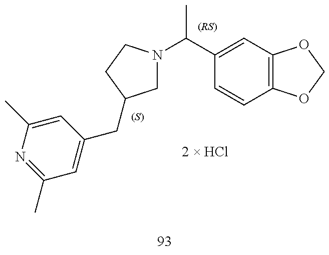 | 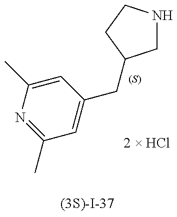 | CAS 3162-29- 6 | — |
| 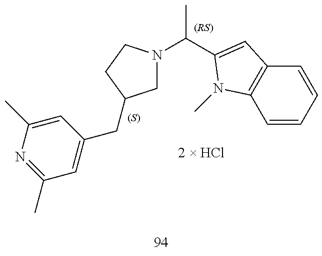 | 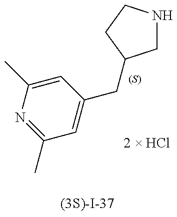 | CAS 16498- 68-3 | — |
| 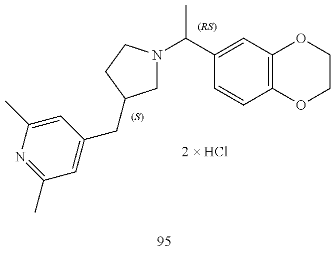 | 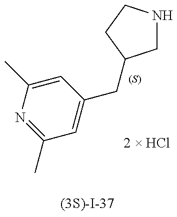 | CAS 2879-20- 1 | — |
| 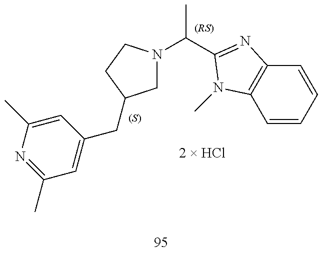 | 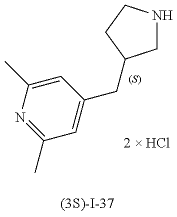 | CAS 942-25-6 | Solvent: THF |
| 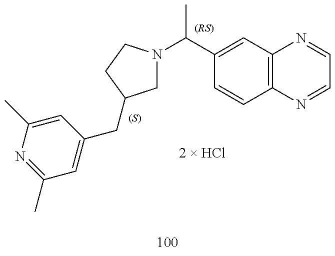 | 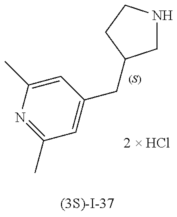 | CAS 83570- 42-7 | — |
| 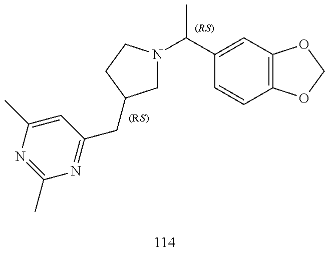 | I-104 | CAS 3162-29- 6 | Solvent: EtOH |
| 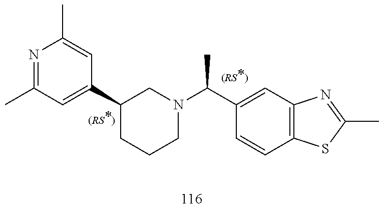 | I-15 | CAS 20077- 88-7 | Solvent: 1,2- dichoroethane |
| 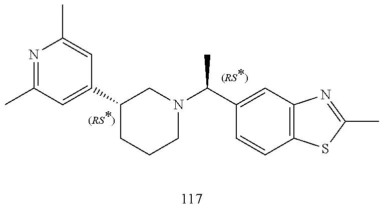 | I-15 | CAS 20077- 88-7 | Solvent: 1,2- dichoroethane |
| 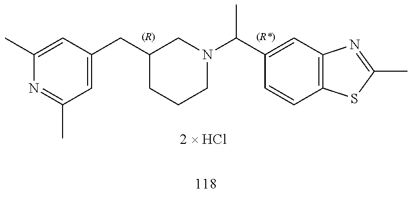 | 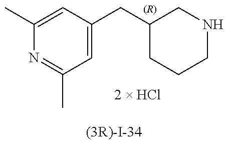 | CAS 20077- 88-7 | Solvent: 1,2- dichoroethane |
| 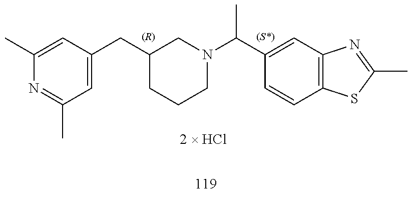 | 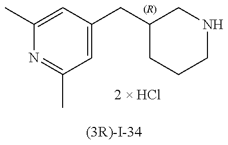 | CAS 20077- 88-7 | Solvent: 1,2- dichoroethane |
| 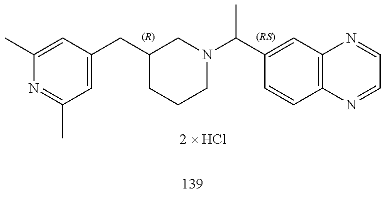 | 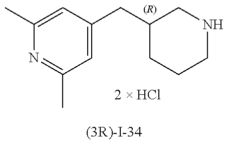 | CAS 83570- 42-7 | — |
| 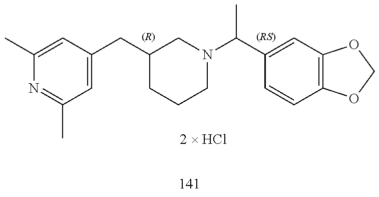 | 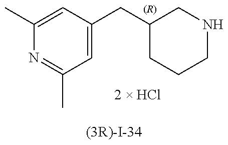 | CAS 3162-29- 6 | — |
| 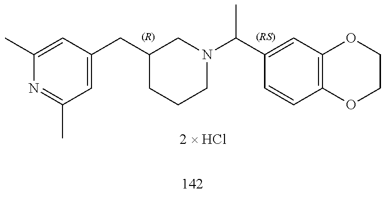 | 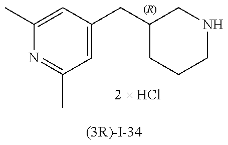 | CAS 2879-20- 1 | — |
| 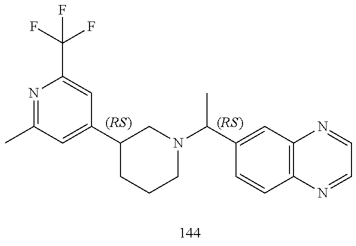 [SMILES: Cc1cc(C2CCCN(C(C)c3ccc4nccnc4c3)C2)cc(C(F)(F)F)n1] | 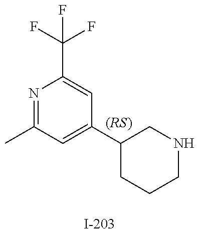 [SMILES: Cc1cc(C2CCCNC2)cc(C(F)(F)F)n1] | CAS 83570- 42-7 | Solvent. 1,2- dichoroethane; no Et₃N used |
| 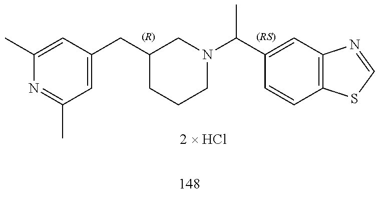 | 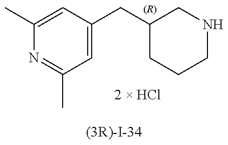 | CAS 90347- 90-3 | — |
| 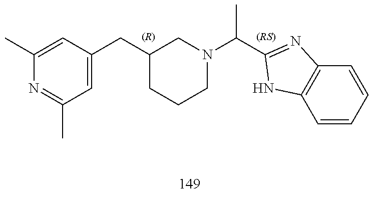 | 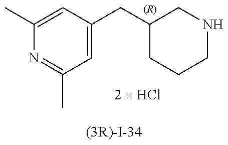 | CAS 18773- 95-0 | — |
| 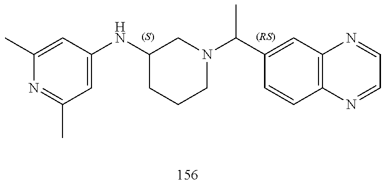 | I-19 | CAS 83570- 42-7 | Solvent: THF No NEt₃ |

### E31. Preparation of Product 120

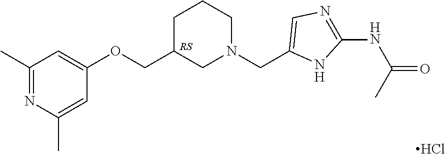 [SMILES: CC(=O)Nc1ncc(CN2CCCC(COc3cc(C)nc(C)c3)C2)[nH]1 ; Cl]

Sodium cyanoborohydride (28.19 mg, 0.52 mmol) was added to a stirred solution of N-(5-formyl-1H-imidazol-2-yl)acetamide ([917919-66-5], 66 mg, 0.259 mmol), 1-23 (68.36 mg, 0.31 mmol) and acetic acid (0.0296 mL, 0.52 mmol) in MeOH (7 mL) at rt for 18 h. The solvents were evaporated in vacuo. The product was purified by RP column chromatography (silica gel; eluent from 81% 25 mM NH₄HCO₃-19% ACN-MeOH (1:1) to 45% 25 mM NH₄HCO₃-55% ACN-MeOH (1:1)). The desired fractions were collected and concentrated in vacuo to yield a yellow oil, which was dissolved in DCM and treated with HCl (4N in dioxane, 30.75 mL), followed by trituration with DIPE to yield product 120 (36.7 mg, 36%) as a white solid.

### Preparation of Products 121-125

The following compounds were prepared following a reductive amination procedure like the one described for the preparation of product 11 starting from the corresponding Boc-protected intermediate amine which was first deprotected by treatment with HCl (6M in iPr) and then reacted with the aldehyde intermediates using triethyl amine and sodium triacetoxyborohydride in 2-tethyltetrahydrofuran.

| | BOC-PROTECTED INTERMEDIATE | INTERMEDIATE |
|---|---|---|
| PRODUCT | AMINE | ALDEHYDE |
| 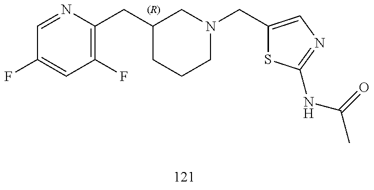 | 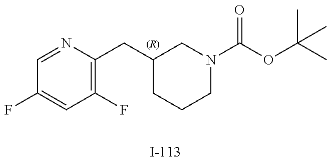 | I-12 |
| 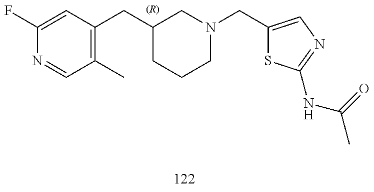 | 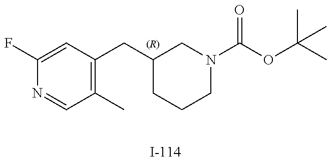 | I-12 |
| 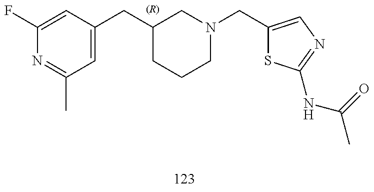 | 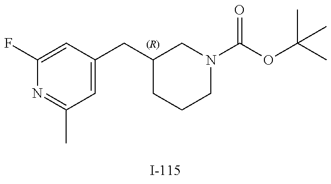 | I-12 |
| 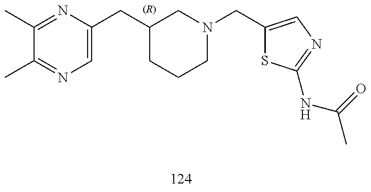 | 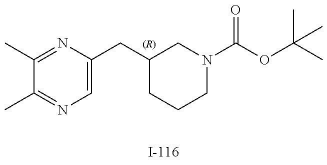 | I-12 |
| 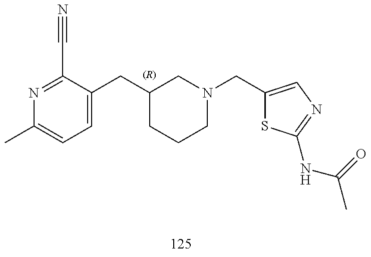 | 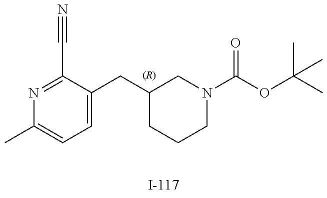 | I-12 |

### E32. Preparation of Products 133-137

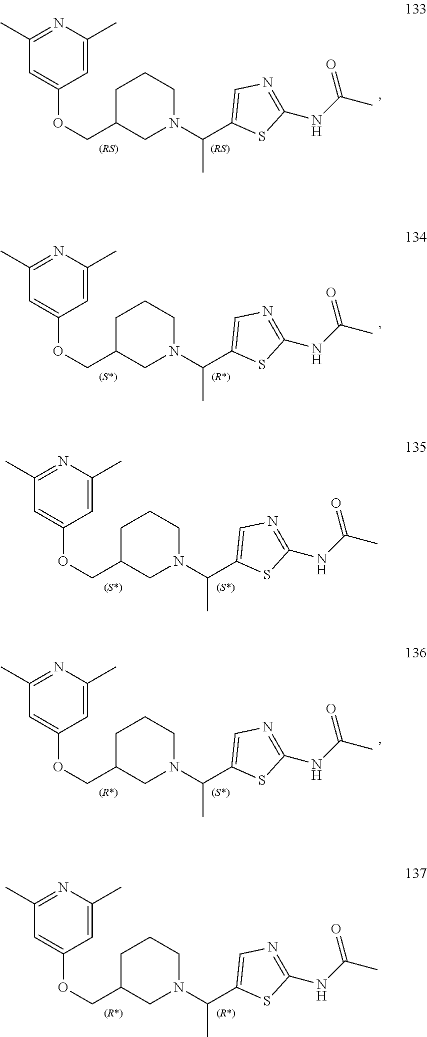

To a solution of intermediate 23 (110 mg, 0.5 mmol) in anhydrous DCM (1.5 mL), intermediate 12 (127 mg, 0.75 mmol) and titanium tetraisopropoxide (0.22 mL, 0.75 mmol) were added and the reaction mixture was stirred at rt for 18 h. Then, the reaction was cooled to 0° C. and methylmagnesium bromide (1.78 mL, 2.5 mmol, 1.4 M in THF) was added dropwise followed by anhydrous THF (1.5 mL) and the reaction mixture was stirred at 0° C. for 5 min and at rt for 4 h. Then NH₄Cl (aq. sat. soltn.) and DCM were added. The organic layer was separated, dried (MgSO₄), filtered and the solvent evaporated in vacuo. The residue was purified by flash column chromatography (silica; MeOH/DCM (9:1) in DCM 0/100 to 100/0). The desired fractions were collected to yield product 133 (126 mg, 64%).

Product 133 (67 mg) was subjected to preparative SFC (stationary phase: Chiralpak® Diacel AD 20×250 mm, mobile phase: CO₂, MeOH+0.4 iPrNH₂) yielding product 134 (9.4 mg), product 135 (10.2 mg) and a mixture of product 136 and product 137 which was was subjected to preparative SFC (stationary phase: Chiralpak® Diacel AD 20×250 mm, mobile phase: CO₂, MeOH+0.4 iPrNH₂) yielding product 136 (10 mg) and product 137 (10.2 mg).

### E33. Preparation of Product 138

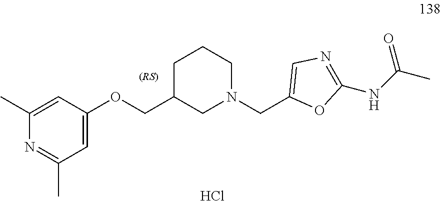 [SMILES: CC(=O)Nc1ncc(CN2CCCC(COc3cc(C)nc(C)c3)C2)o1 ; Cl]

To acetyl chloride (0.029 mL, 0.4 mmol) was added dropwise to a solution of intermediate 118 (106 mg, 0.33 mmol) and pyridine (132 mg, 1.67 mmol) in DCM at 0° C. The mixture was stirred overnight at rt and then cooled to 0° C. and additional acetyl chloride (1 eq) was added. The mixture was stirred at rt for 2 days. The volatiles were evaporated in vacuo. Toluene was added and the mixture was concentrated in vacuo. The residue was purificated by reverse phase chromatography 90% [25 mM NH₄HCO₃]-10% [ACN:MeOH 1:1] to 54% [25 mM NH₄HCO₃]-46% [ACN:MeOH 1:1]. The volatiles were evaporated in vacuo and ACN (3×10 mL) was added and concentrated yielding product 138 as a free base (77 mg, 62%). This was taken up in DCM (5 mL) and HCl (0.053 mL, 0.215 mmol, 4N in 1,4-dioxane) was added. The Et₂O was wadded and the solvent was evaporated in vacuo. The residue thus obtained was treated with diisopropyl ether to give a solid that was filtered and dried affording product 138 (65 mg, 47%, HCl salt) was a white solid.

### E34. Preparation of Products 167 and 168

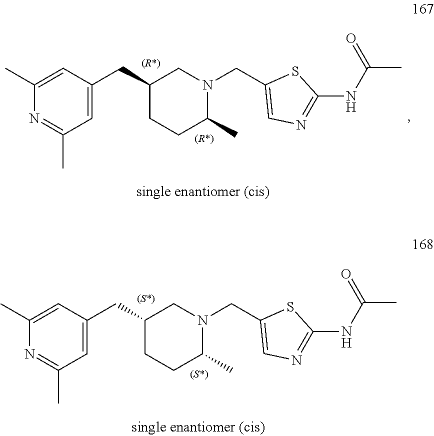

Product 166 (196 mg) was subjected to chiral SFC (stationary phase: CHIRALPAK® AD-H 5 μm 250*30 mm, mobile phase: 70% CO₂, 30% iPOH (0.3% iPrNH₂)) yielding product 167 (47 mg) and impure product 168 (51 mg). Impure product 168 (51 mg) was subjected to chiral SFC (stationary phase: CHIRALPAK® AD-H 5 μm 250*30 mm, mobile phase: 70% CO₂, 30% iPOH (0.3% iPrNH₂)) yielding product 168 (31 mg).

### E35. Preparation of Products 170 and 171

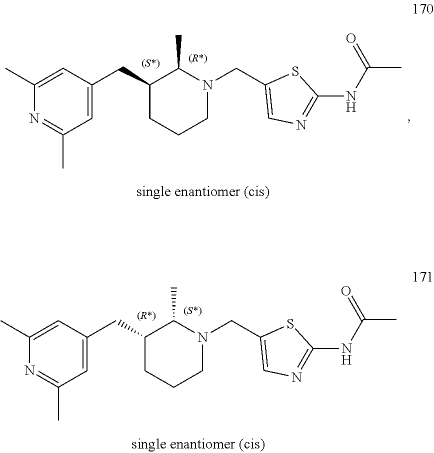

Product 169 (52 mg) was subjected to chiral SFC (stationary phase: CHIRALPAK® AD-H 5 μm 250*30 mm, mobile phase: 55% CO₂, 45% EtOH (0.3% iPrNH₂)) yielding product 170 (18 mg) and product 171 (20 mg).

### E36. Preparation of Product 172

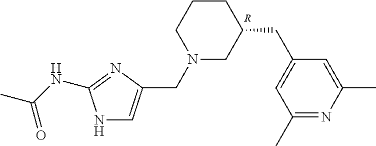

N-(5-Formyl-1H-imidazol-2-yl)-acetamide ([917919-66-5], 52 mg, 0.34 mmol) followed by DMF (0.3 mL) were added to a stirred solution of (3R)—I-34 (71 mg, 0.35 mmol) in DCE (1.4 mL) in a sealed tube and under N₂. The mixture was stirred at rt for 5 min and then sodium triacetoxyborohydride (205 mg, 0.97 mmol) was added. The mixture was stirred at rt for 60 h. The mixture was treated with sat NaHCO₃ and extracted with DCM. The organic layer was separated, dried (MgSO₄), filtered and the solvents evaporated in vacuo. The crude product was purified by RP HPLC (stationary phase: C18 XBridge 30×100 mm 5 μm; mobile phase: gradient from 90% NH₄HCO₃ 0.25% solution in water, 10% CH₃CN to 65% NH₄HCO₃ 0.25% solution in water, 35% CH₃CN). The desired fractions were collected and extracted with EtOAc. The organic layer was separated, dried (MgSO₄), filtered and the solvents evaporated in vacuo to yield product 172 (52 mg, 44%) as a colourless oil that precipitate upon standing.

### E37. Preparation of Product 173

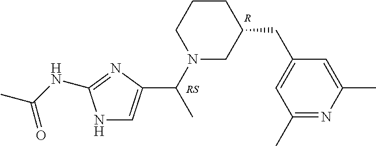

N-(5-Formyl-1H-imidazol-2-yl)-acetamide ([917919-66-5], 87 mg, 0.43 mmol) was added dropwise to a stirred suspension of (3R)—I-34 (87 mg, 0.43 mmol) and Ti(iPrO)₄ (400 μL, 1.37 mmol) in DCM (1.6 mL) in a sealed tube and under N₂. The mixture was stirred at rt for 2 h, then it was cooled to 0° C. and methylmagnesium bromide (1.4 M in THF, 1.6 mL, 2.24 mmol) was added dropwise. The mixture was stirred at rt for 16 h, then it was treated with sat NH₄Cl and DCM and filtered through a Celite® pad and washed with additional DCM. The filtrate was extracted with additional DCM. The organic layer was separated, dried (MgSO₄), filtered and the solvents evaporated in vacuo. The crude product was purified by RP HPLC (stationary phase: C18 XBridge 30×100 mm 5 μm; mobile phase: gradient from 80% NH₄HCO₃ 0.25% solution in water, 20% CH₃CN to 60% NH₄HCO₃ 0.25% solution in water, 40% CH₃CN). The desired fractions were collected and extracted with EtOAc. The organic layer was separated, dried (MgSO₄), filtered and the solvents evaporated in vacuo to yield product 173 (13 mg, 9%) as a pale yellow oil.

### E38. Preparation of Product 174

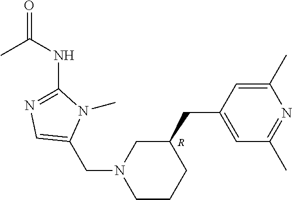

TFA (0.06 mL, 5 eq) was added to a stirred solution of I-225 (65 mg, 0.16 mmol) in DCM (1.2 mL) in a sealed tube and under N₂. The mixture was stirred at rt for 17 h. Then additional TFA (0.12 mL, 10 eq) was added and the mixture was stirred at rt for 24 h. The solvent was evaporated in vacuo and the crude was treated with DCM (1.6 mL), cooled at 0° C. and Et₃N (120 μL) and acetyl chloride (15 μL, 0.21 mmol) were added. The mixture was stirred at 0° C. for 5 min and at rt for 2.5 h. The mixture was treated with sat NaHCO₃ and extracted with more DCM. The organic layer was separated, dried (MgSO₄), filtered and the solvent evaporated in vacuo. The crude was purified by RP HPLC (stationary phase: C18 XBridge 30×100 mm 5 μm; mobile phase: gradient from 80% NH₄HCO₃ 0.25% solution in water, 20% CH₃CN to 60% NH₄HCO₃ 0.25% solution in water, 40% CH₃CN). The desired fractions were collected and extracted with EtOAc. The organic layer was separated, dried (MgSO₄), filtered and the solvent evaporated in vacuo to yield product 174 (8 mg, 14%) as a pale purple oil.

The following compounds were prepared following the methods exemplified in the Experimental Part. In case no salt form is indicated, the compound was obtained as a free base. ‘Ex. No.’ refers to the Example number according to which protocol the compound was synthesized. ‘Co. No.’ means compound number.

TABLE 1

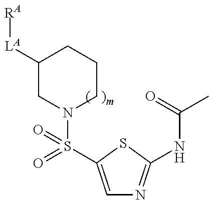

| Co. no. | Exp no. | m | L^{A} | R^{A} | Stereochem/Salt |
|---|---|---|---|---|---|
| 1 | E1 | 1 | O | 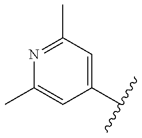 [SMILES: *c1cc(C)nc(C)c1] | 3-RS |
| 2 | E2 | 1 | O | 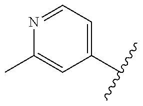 [SMILES: *c1ccnc(C)c1] | 3-RS |
| 3 | E3 | 0 | bond | 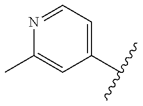 [SMILES: *c1ccnc(C)c1] | 3-RS |
| 4 | E4 | 0 | bond | 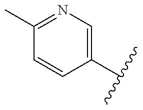 [SMILES: *c1ccc(C)nc1] | 3-RS |
| 33 | E29 | 1 | CH₂ | 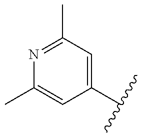 [SMILES: *c1cc(C)nc(C)c1] | 3-R |

TABLE 2

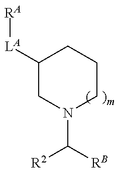

| | Exp | | | | | | |
|---|---|---|---|---|---|---|---|
| Co. no. | no. | m | L^{A} | R^{A} | R² | R^{B} | Stereochem/Salt |
| 5^{#} | E5 | 1 | bond | 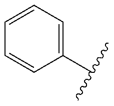 [SMILES: *c1ccccc1] | H | b-1 (Q¹ = CH, Q² = S, R^{1b} = H, R^{2b} = CH₃) | 3-RS |
| 6 | E6 | 1 | Bond | 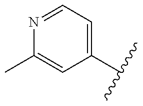 [SMILES: *c1ccnc(C)c1] | H | b-1 (Q¹ = CH, Q² = S, R^{1b} = H, R^{2b} = CH₃) | 3-RS |
| 7 | E7 | 1 | Bond | 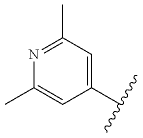 [SMILES: *c1cc(C)nc(C)c1] | H | b-1 (Q¹ = CH, Q² = S, R^{1b} = H, R^{2b} = CH₃) | 3-RS |
| 8 | E8 | 1 | NH | 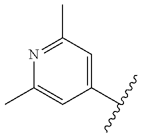 [SMILES: *c1cc(C)nc(C)c1] | H | b-1 (Q¹ = CH, Q² = S, R^{1b} = H, R^{2b} = CH₃) | 3-R |
| 9 | E9 | 1 | NH | 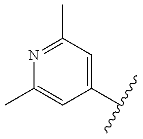 [SMILES: *c1cc(C)nc(C)c1] | H | b-1 (Q¹ = CH, Q² = S, R^{1b} = H, R^{2b} = CH₃) | 3-S |
| 10 | E10 | 1 | NH | 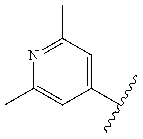 [SMILES: *c1cc(C)nc(C)c1] | H | b-4 | 3-R |
| 11 | E11 | 1 | NH | 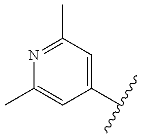 [SMILES: *c1cc(C)nc(C)c1] | CH₃ | b-4 | 3-R |
| 12 | E12 | 1 | NH | 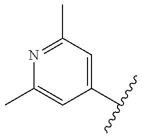 [SMILES: *c1cc(C)nc(C)c1] | H | b-4 | 3-S |
| 13^{#} | E13 | 1 | O | 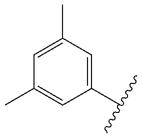 [SMILES: *c1cc(C)cc(C)c1] | H | b-1 (Q¹ = CH, Q² = S, R^{1b} = H, R^{2b} = CH₃) | 3-RS |
| 14 | E14 | 1 | O | 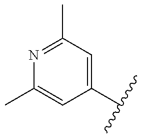 [SMILES: *c1cc(C)nc(C)c1] | H | b-1 (Q¹ = CH, Q² = S, R^{1b} = H, R^{2b} = CH₃) | 3-RS |
| 15 | E15 | 1 | O | 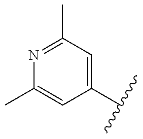 [SMILES: *c1cc(C)nc(C)c1] | H | b-1 (Q¹ = CH, Q² = S, R^{1b} = H, R^{2b} = CH₃) | 3-R |
| 16 | E16 | 1 | O | 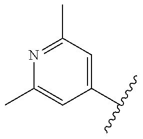 [SMILES: *c1cc(C)nc(C)c1] | H | b-1 (Q¹ = CH, Q² = S, R^{1b} = H, R^{2b} = CH₃) | 3-S |
| 17 | E17 | 1 | O | 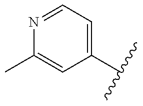 [SMILES: *c1ccnc(C)c1] | H | b-1 (Q¹ = CH, Q² = S, R^{1b} = H, R^{2b} = CH₃) | 3-RS |
| 18 | E18 | 1 | O | 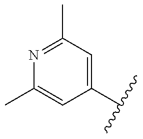 [SMILES: *c1cc(C)nc(C)c1] | H | b-4 | 3-R |
| 19 | E19 | 1 | O | 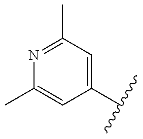 [SMILES: *c1cc(C)nc(C)c1] | CH₃ | b-4 | 1′-RS, 3-R |
| 20 | E20 | 1 | OCH₂ | 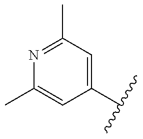 [SMILES: *c1cc(C)nc(C)c1] | H | b-1 (Q¹ = CH, Q² = S, R^{1b} = H, R^{2b} = CH₃) | 3-RS |
| 21 | E21 | 1 | CH₂O | 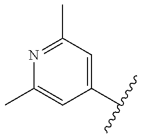 [SMILES: *c1cc(C)nc(C)c1] | H | b-1 (Q¹ = CH, Q² = S, R^{1b} = H, R^{2b} = CH₃) | 3-RS |
| 22 | E22 | 0 | NH | 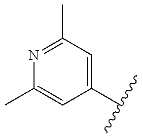 [SMILES: *c1cc(C)nc(C)c1] | H | b-1 (Q¹ = CH, Q² = S, R^{1b} = H, R^{2b} = CH₃) | 3-R |
| 23 | E23 | 0 | NH | 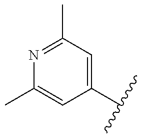 [SMILES: *c1cc(C)nc(C)c1] | H | b-1 (Q¹ = CH, Q² = S, R^{1b} = H, R^{2b} = CH₃) | 3-S |
| 24 | E24 | 1 | CH₂ | 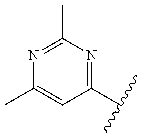 [SMILES: *c1cc(C)nc(C)n1] | H | b-1 (Q¹ = CH, Q² = S, R^{1b} = H, R^{2b} = CH₃) | 3-RS |
| 25 | E24 | 1 | CH₂ | 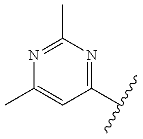 [SMILES: *c1cc(C)nc(C)n1] | H | b-1 (Q¹ = CH, Q² = S, R^{1b} = H, R^{2b} = CH₃) | 3-R* |
| 26 | E24 | 1 | CH₂ | 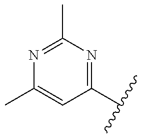 [SMILES: *c1cc(C)nc(C)n1] | H | b-1 (Q¹ = CH, Q² = S, R^{1b} = H, R^{2b} = CH₃) | 3-S* |
| 27 | E25 | 1 | CH₂ | 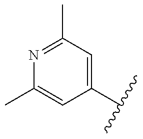 [SMILES: *c1cc(C)nc(C)c1] | H | b-1 (Q¹ = CH, Q² = S, R^{1b} = H, R^{2b} = CH₃) | 3-RS |
| 28 | E25 | 1 | CH₂ | 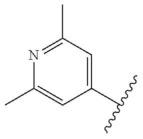 [SMILES: *c1cc(C)nc(C)c1] | H | b-1 (Q¹ = CH, Q² = S, R^{1b} = H, R^{2b} = CH₃) | 3-R |
| 29 | E25 | 1 | CH₂ | 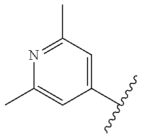 [SMILES: *c1cc(C)nc(C)c1] | H | b-1 (Q¹ = CH, Q² = S, R^{1b} = H, R^{2b} = CH₃) | 3-S |
| 30 | E26 | 0 | CH₂ | 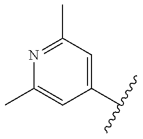 [SMILES: *c1cc(C)nc(C)c1] | H | b-1 (Q¹ = CH, Q² = S, R^{1b} = H, R^{2b} = CH₃) | 3-RS |
| 31 | E27 | 1 | NH | 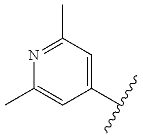 [SMILES: *c1cc(C)nc(C)c1] | H | b-1 (Q¹ = CH, Q² = S, R^{1b} = H, R^{2b} = CH₃) | 3-RS |
| 32 | E28 | 1 | NCH₃ | 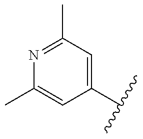 [SMILES: *c1cc(C)nc(C)c1] | H | b-1 (Q¹ = CH, Q² = S, R^{1b} = H, R^{2b} = CH₃) | 3-RS |
| 34 | E20 | 0 | Bond | 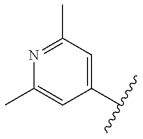 [SMILES: *c1cc(C)nc(C)c1] | H | b-1 (Q¹ = CH, Q² = S, R^{1b} = H, R^{2b} = CH₃) | 3-RS |
| 35 | E20 | 0 | CH₂ | 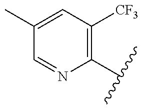 [SMILES: *c1ncc(C)cc1C(F)(F)F] | H | b-1 (Q¹ = CH, Q² = S, R^{1b} = H, R^{2b} = CH₃) | 3-S |
| 36 | E20 | 0 | CH₂ | 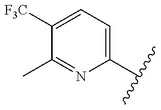 [SMILES: *c1ccc(C(F)(F)F)c(C)n1] | H | b-1 (Q¹ = CH, Q² = S, R^{1b} = H, R^{2b} = CH₃) | 3-S |
| 37 | E20 | 0 | CH₂ | 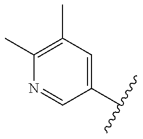 [SMILES: *c1cnc(C)c(C)c1] | H | b-1 (Q¹ = CH, Q² = S, R^{1b} = H, R^{2b} = CH₃) | 3-S |
| 38 | E20 | 0 | CH₂ | 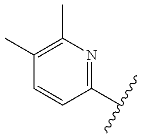 [SMILES: *c1ccc(C)c(C)n1] | H | b-1 (Q¹ = CH, Q² = S, R^{1b} = H, R^{2b} = CH₃) | 3-R |
| 39 | E20 | 0 | CH₂ | 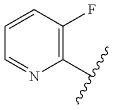 [SMILES: *c1ncccc1F] | H | b-1 (Q¹ = CH, Q² = S, R^{1b} = H, R^{2b} = CH₃) | 3-R |
| 40 | E20 | 0 | CH₂ | 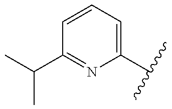 [SMILES: *c1cccc(C(C)C)n1] | H | b-1 (Q¹ = CH, Q² = S, R^{1b} = H, R^{2b} = CH₃) | 3-R |
| 41 | E20 | 0 | CH₂ | 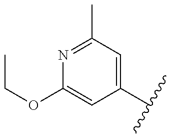 [SMILES: *c1cc(C)nc(OCC)c1] | H | b-1 (Q¹ = CH, Q² = S, R^{1b} = H, R^{2b} = CH₃) | 3-S |
| 42 | E20 | 0 | CH₂ | 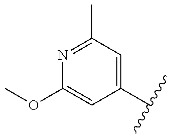 [SMILES: *c1cc(C)nc(OC)c1] | H | b-1 (Q¹ = CH, Q² = S, R^{1b} = H, R^{2b} = CH₃) | 3-S |
| 43 | E20 | 0 | CH₂ | 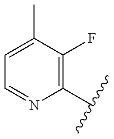 [SMILES: *c1nccc(C)c1F] | H | b-1 (Q¹ = CH, Q² = S, R^{1b} = H, R^{2b} = CH₃) | 3-R |
| 44 | E30 | 1 | CH₂ | 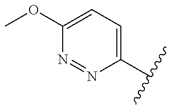 [SMILES: *c1ccc(OC)nn1] | H | b-1 (Q¹ = CH, Q² = S, R^{1b} = H, R^{2b} = CH₃) | 3-R |
| 45 | E20 | 0 | CH₂ | 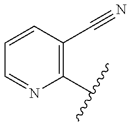 [SMILES: *c1ncccc1C#N] | H | b-1 (Q¹ = CH, Q² = S, R^{1b} = H, R^{2b} = CH₃) | 3-R |
| 46 | E20 | 0 | CH₂ | 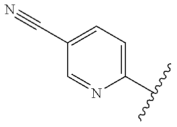 [SMILES: *c1ccc(C#N)cn1] | H | b-1 (Q¹ = CH, Q² = S, R^{1b} = H, R^{2b} = CH₃) | 3-R |
| 47 | E20 | 0 | CH₂ | 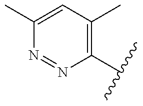 | H | b-1 (Q¹ = CH, Q² = S, R^{1b} = H, R^{2b} = CH₃) | 3-R |
| 48 | E20 | 0 | CH₂ | 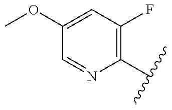 [SMILES: *c1ncc(OC)cc1F] | H | b-1 (Q¹ = CH, Q² = S, R^{1b} = H, R^{2b} = CH₃) | 3-R |
| 49 | E20 | 0 | CH₂ | 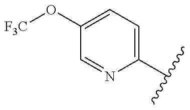 [SMILES: *c1ccc(OC(F)(F)F)cn1] | H | b-1 (Q¹ = CH, Q² = S, R^{1b} = H, R^{2b} = CH₃) | 3-R |
| 50 | E20 | 0 | CH₂ | 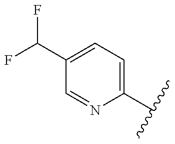 [SMILES: *c1ccc(C(F)F)cn1] | H | b-1 (Q¹ = CH, Q² = S, R^{1b} = H, R^{2b} = CH₃) | 3-R |
| 51 | E20 | 0 | CH₂ | 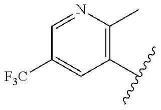 [SMILES: *c1cc(C(F)(F)F)cnc1C] | H | b-1 (Q¹ = CH, Q² = S, R^{1b} = H, R^{2b} = CH₃) | 3-S |
| 52 | E20 | 0 | CH₂ | 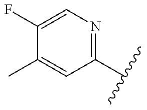 [SMILES: *c1cc(C)c(F)cn1] | H | b-1 (Q¹ = CH, Q² = S, R^{1b} = H, R^{2b} = CH₃) | 3-R |
| 53 | E20 | 0 | CH₂ | 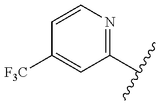 [SMILES: *c1cc(C(F)(F)F)ccn1] | H | b-1 (Q¹ = CH, Q² = S, R^{1b} = H, R^{2b} = CH₃) | 3-R |
| 54 | E20 | 0 | CH₂ | 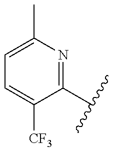 [SMILES: *c1nc(C)ccc1C(F)(F)F] | H | b-1 (Q¹ = CH, Q² = S, R^{1b} = H, R^{2b} = CH₃) | 3-R |
| 55 | E20 | 0 | CH₂ | 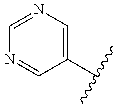 [SMILES: *c1cncnc1] | H | b-1 (Q¹ = CH, Q² = S, R^{1b} = H, R^{2b} = CH₃) | 3-S |
| 56 | E20 | 0 | CH₂ | 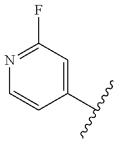 [SMILES: *c1ccnc(F)c1] | H | b-1 (Q¹ = CH, Q² = S, R^{1b} = H, R^{2b} = CH₃) | 3-S |
| 57 | E20 | 0 | CH₂ | 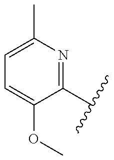 [SMILES: *c1nc(C)ccc1OC] | H | b-1 (Q¹ = CH, Q² = S, R^{1b} = H, R^{2b} = CH₃) | 3-R |
| 58 | E20 | 0 | CH₂ | 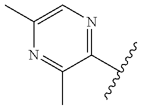 [SMILES: *c1ncc(C)nc1C] | H | b-1 (Q¹ = CH, Q² = S, R^{1b} = H, R^{2b} = CH₃) | 3-R |
| 59 | E20 | 0 | CH₂ | 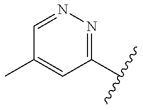 [SMILES: *c1cc(C)cnn1] | H | b-1 (Q¹ = CH, Q² = S, R^{1b} = H, R^{2b} = CH₃) | 3-R |
| 60 | E20 | 0 | CH₂ | 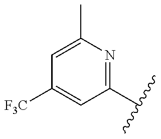 [SMILES: *c1cc(C(F)(F)F)cc(C)n1] | H | b-1 (Q¹ = CH, Q² = S, R^{1b} = H, R^{2b} = CH₃) | 3-R |
| 61 | E20 | 0 | CH₂ | 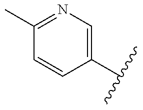 [SMILES: *c1ccc(C)nc1] | H | b-1 (Q¹ = CH, Q² = S, R^{1b} = H, R^{2b} = CH₃) | 3-S |
| 62 | E20 | 0 | CH₂ | 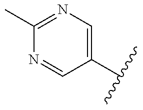 [SMILES: *c1cnc(C)nc1] | H | b-1 (Q¹ = CH, Q² = S, R^{1b} = H, R^{2b} = CH₃) | 3-S |
| 63 | E20 | 0 | CH₂ | 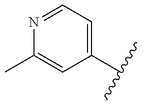 [SMILES: *c1ccnc(C)c1] | H | b-1 (Q¹ = CH, Q² = S, R^{1b} = H, R^{2b} = CH₃) | 3-S |
| 64 | E20 | 0 | CH₂ | 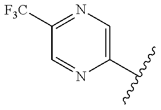 [SMILES: *c1cnc(C(F)(F)F)cn1] | H | b-1 (Q¹ = CH, Q² = S, R^{1b} = H, R^{2b} = CH₃) | 3-R |
| 65 | E20 | 0 | CH₂ | 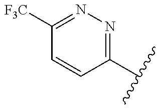 [SMILES: *c1ccc(C(F)(F)F)nn1] | H | b-1 (Q¹ = CH, Q² = S, R^{1b} = H, R^{2b} = CH₃) | 3-R |
| 66 | E20 | 0 | CH₂ | 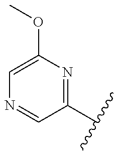 [SMILES: *c1cncc(OC)n1] | H | b-1 (Q¹ = CH, Q² = S, R^{1b} = H, R^{2b} = CH₃) | 3-R |
| 67 | E20 | 0 | CH₂ | 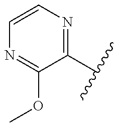 [SMILES: *c1nccnc1OC] | H | b-1 (Q¹ = CH, Q² = S, R^{1b} = H, R^{2b} = CH₃) | 3-R |
| 68 | E20 | 0 | CH₂ | 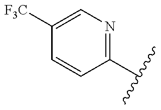 [SMILES: *c1ccc(C(F)(F)F)cn1] | H | b-1 (Q¹ = CH, Q² = S, R^{1b} = H, R^{2b} = CH₃) | 3-R |
| 69 | E20 | 0 | CH₂ | 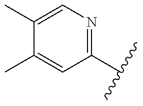 [SMILES: *c1cc(C)c(C)cn1] | H | b-1 (Q¹ = CH, Q² = S, R^{1b} = H, R^{2b} = CH₃) | 3-R |
| 70 | E20 | 0 | CH₂ | 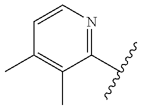 [SMILES: *c1nccc(C)c1C] | H | b-1 (Q¹ = CH, Q² = S, R^{1b} = H, R^{2b} = CH₃) | 3-R |
| 71 | E20 | 0 | CH₂ | 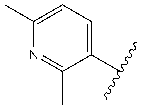 [SMILES: *c1ccc(C)nc1C] | H | b-1 (Q¹ = CH, Q² = S, R^{1b} = H, R^{2b} = CH₃) | 3-S |
| 72 | E20 | 0 | CH₂ | 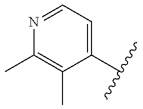 [SMILES: *c1ccnc(C)c1C] | H | b-1 (Q¹ = CH, Q² = S, R^{1b} = H, R^{2b} = CH₃) | 3-S |
| 73 | E20 | 0 | CH₂ | 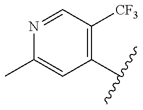 [SMILES: *c1cc(C)ncc1C(F)(F)F] | H | b-1 (Q¹ = CH, Q² = S, R^{1b} = H, R^{2b} = CH₃) | 3-S |
| 74 | E20 | 0 | CH₂ | 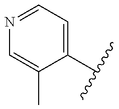 [SMILES: *c1ccncc1C] | H | b-1 (Q¹ = CH, Q² = S, R^{1b} = H, R^{2b} = CH₃) | 3-S |
| 75 | E20 | 0 | CH₂ | 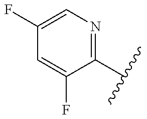 [SMILES: *c1ncc(F)cc1F] | H | b-1 (Q¹ = CH, Q² = S, R^{1b} = H, R^{2b} = CH₃) | 3-R |
| 76 | E20 | 0 | CH₂ | 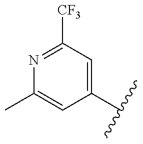 [SMILES: *c1cc(C)nc(C(F)(F)F)c1] | H | b-1 (Q¹ = CH, Q² = S, R^{1b} = H, R^{2b} = CH₃) | 3-S |
| 77 | E20 | 0 | CH₂ | 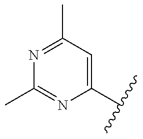 [SMILES: *c1cc(C)nc(C)n1] | H | b-1 (Q¹ = CH, Q² = S, R^{1b} = H, R^{2b} = CH₃) | 3-R |
| 78 | E11 | 0 | CH₂ | 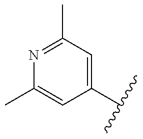 [SMILES: *c1cc(C)nc(C)c1] | CH₃ | b-9 (Q¹ = N, R^{3b} = H) | 1′-RS, 3-S |
| 79 | E20 | 0 | CH₂ | 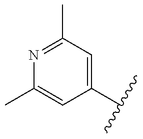 [SMILES: *c1cc(C)nc(C)c1] | H | b-9 (Q¹ = N, R^{3b} = H) | 3-S |
| 80 | E20 | 0 | CH₂ | 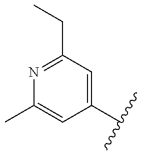 [SMILES: *c1cc(C)nc(CC)c1] | H | b-1 (Q¹ = CH, Q² = S, R^{1b} = H, R^{2b} = CH₃) | 3-S •HCl |
| 81 | E20 | 0 | CH₂O | 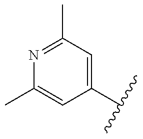 [SMILES: *c1cc(C)nc(C)c1] | H | b-1 (Q¹ = CH, Q² = S, R^{1b} = H, R^{2b} = CH₃) | 3-RS |
| 82 | E20 | 0 | CH₂ | 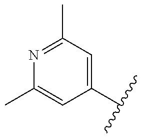 [SMILES: *c1cc(C)nc(C)c1] | H | b-11 (R^{4b} = CH₃) | 3-S |
| 83 | E20 | 0 | CH₂ | 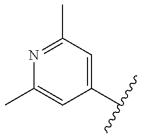 [SMILES: *c1cc(C)nc(C)c1] | H | b-11 (R^{4b} = H) | 3-S |
| 84 | E20 | 0 | CH₂ | 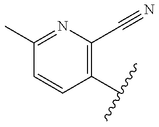 [SMILES: *c1ccc(C)nc1C#N] | H | b-1 (Q¹ = CH, Q² = S, R^{1b} = H, R^{2b} = CH₃) | 3-S |
| 85 | E20 | 0 | CH₂ | 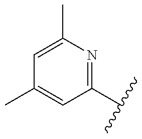 [SMILES: *c1cc(C)cc(C)n1] | H | b-1 (Q¹ = CH, Q² = S, R^{1b} = H, R^{2b} = CH₃) | 3-R |
| 86 | E20 | 0 | CH₂ | 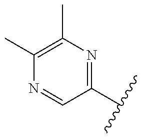 [SMILES: *c1cnc(C)c(C)n1] | H | b-1 (Q¹ = CH, Q² = S, R^{1b} = H, R^{2b} = CH₃) | 3-R |
| 87 | E11 | 0 | CH₂ | 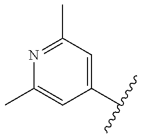 [SMILES: *c1cc(C)nc(C)c1] | CH₃ | b-11 (R^{4b} = CH₃) | 1′-RS, 3-S |
| 88 | E11 | 0 | CH₂ | 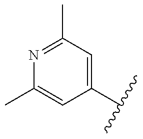 [SMILES: *c1cc(C)nc(C)c1] | CH₃ | b-11 (R^{4b} = H) | 1′-RS, 3-S |
| 89 | E20 | 0 | OCH₂ | 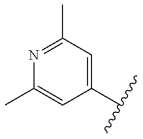 [SMILES: *c1cc(C)nc(C)c1] | H | b-1 (Q¹ = CH, Q² = S, R^{1b} = H, R^{2b} = CH₃) | 3-RS |
| 90 | E20 | 0 | CH₂ | 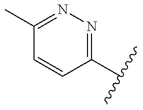 [SMILES: *c1ccc(C)nn1] | H | b-1 (Q¹ = CH, Q² = S, R^{1b} = H, R^{2b} = CH₃) | 3-R |
| 91 | E20 | 0 | CH₂ | 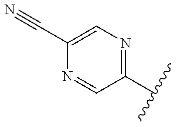 [SMILES: *c1cnc(C#N)cn1] | H | b-1 (Q¹ = CH, Q² = S, R^{1b} = H, R^{2b} = CH₃) | 3-R |
| 92 | E20 | 0 | CH₂ | 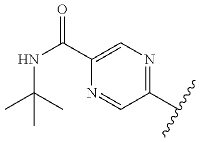 [SMILES: *c1cnc(C(=O)NC(C)(C)C)cn1] | H | b-1 (Q¹ = CH, Q² = S, R^{1b} = H, R^{2b} = CH₃) | 3-R |
| 93 | E11 | 0 | CH₂ | 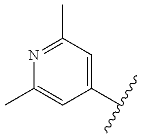 [SMILES: *c1cc(C)nc(C)c1] | CH₃ | b-2 | 1′-RS, 3-S •2HCl |
| 94 | E11 | 0 | CH₂ | 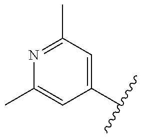 [SMILES: *c1cc(C)nc(C)c1] | CH₃ | b-9 (Q¹ = CH, R^{3b} = CH₃) | 1′-RS, 3-S •2HCl |
| 95 | E11 | 0 | CH₂ | 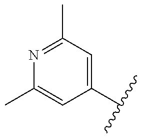 [SMILES: *c1cc(C)nc(C)c1] | CH₃ | b-3 | 1′-RS, 3-S •2HCl |
| 96 | E11 | 0 | CH₂ | 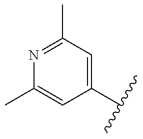 [SMILES: *c1cc(C)nc(C)c1] | CH₃ | b-9 (Q¹ = N, R^{3b} = CH₃) | 1′-RS, 3-S •2HCl |
| 97 | E20 | 0 | CH₂ | 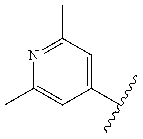 [SMILES: *c1cc(C)nc(C)c1] | H | b-9 (Q¹ = N, R^{3b} = CH₃) | 3-S |
| 98 | E20 | 0 | CH₂ | 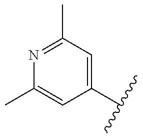 [SMILES: *c1cc(C)nc(C)c1] | H | b-9 (Q¹ = CH, R^{3b} = CH₃) | 3-S |
| 99 | E20 | 0 | CH₂ | 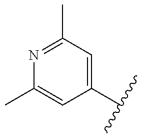 [SMILES: *c1cc(C)nc(C)c1] | H | b-2 | 3-S |
| 100 | E11 | 0 | CH₂ | 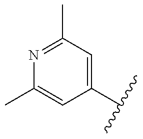 [SMILES: *c1cc(C)nc(C)c1] | CH₃ | b-4 | 1′-RS, 3-S •2HCl |
| 101 | E20 | 0 | CH₂ | 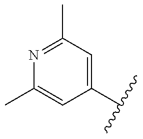 [SMILES: *c1cc(C)nc(C)c1] | H | b-3 | 3-S |
| 102 | E20 | 0 | CH₂ | 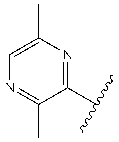 [SMILES: *c1nc(C)cnc1C] | H | b-1 (Q¹ = CH, Q² = S, R^{1b} = H, R^{2b} = CH₃) | 3-R |
| 103 | E20 | 0 | CH₂ | 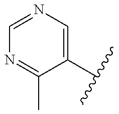 [SMILES: *c1cncnc1C] | H | b-1 (Q¹ = CH, Q² = S, R^{1b} = H, R^{2b} = CH₃) | 3-RS |
| 104 | E20 | 0 | CH₂ | 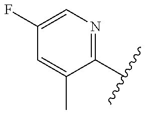 [SMILES: *c1ncc(F)cc1C] | H | b-1 (Q¹ = CH, Q² = S, R^{1b} = H, R^{2b} = CH₃) | 3-RS |
| 105 | E20 | 0 | CH₂ | 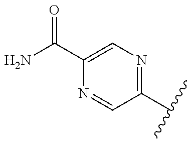 [SMILES: *c1cnc(C(N)=O)cn1] | H | b-1 (Q¹ = CH, Q² = S, R^{1b} = H, R^{2b} = CH₃) | 3-RS |
| 106 | E20 | 0 | CH₂ | 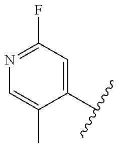 [SMILES: *c1cc(F)ncc1C] | H | b-1 (Q¹ = CH, Q² = S, R^{1b} = H, R^{2b} = CH₃) | 3-RS |
| 107 | E20 | 0 | CH₂ | 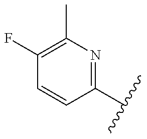 [SMILES: *c1ccc(F)c(C)n1] | H | b-1 (Q¹ = CH, Q² = S, R^{1b} = H, R^{2b} = CH₃) | 3-RS |
| 108 | E20 | 0 | CH₂ | 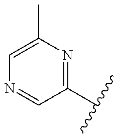 [SMILES: *c1cncc(C)n1] | H | b-1 (Q¹ = CH, Q² = S, R^{1b} = H, R^{2b} = CH₃) | 3-RS |
| 109 | E20 | 0 | CH₂ | 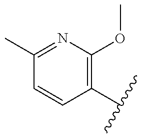 | H | b-1 (Q¹ = CH, Q² = S, R^{1b} = H, R^{2b} = CH₃) | 3-RS |
| 110 | E20 | 0 | CH₂ | 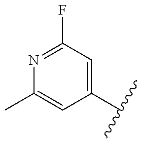 [SMILES: *c1cc(C)nc(F)c1] | H | b-1 (Q¹ = CH, Q² = S, R^{1b} = H, R^{2b} = CH₃) | 3-S |
| 111 | E20 | 0 | CH₂ | 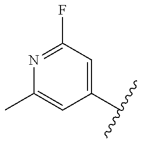 [SMILES: *c1cc(C)nc(F)c1] | H | b-1 (Q¹ = CH, Q² = S, R^{1b} = H, R^{2b} = CH₃) | 3-R |
| 112 | E20 | 0 | CH₂ | 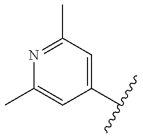 [SMILES: *c1cc(C)nc(C)c1] | H | b-1 (Q¹ = H, Q² = S, R^{1b} = H, R^{2b} = CH₃) | 3-S |
| 113 | E20 | 0 | CH₂ | 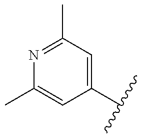 [SMILES: *c1cc(C)nc(C)c1] | H | b-1 (Q¹ = CH, Q² = S, R^{1b} = H, R^{2b} = CH₃) | 3-R |
| 114 | E11 | 0 | CH₂ | 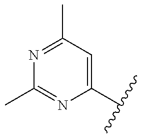 [SMILES: *c1cc(C)nc(C)n1] | CH₃ | b-2 | 1′-RS, 3-RS |
| 115 | E20 | 0 | CH₂ | 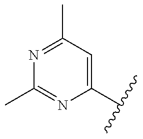 [SMILES: *c1cc(C)nc(C)n1] | H | b-1 (Q¹ = CH, Q² = S, R^{1b} = H, R^{2b} = CH₃) | 3-RS |
| 116 | E11 | 1 | Bond | 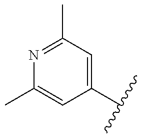 [SMILES: *c1cc(C)nc(C)c1] | CH₃ | b-9 (Q¹ = H, R^{3b} = CH₃) | 1′-RS*, 3-RS* Single diastereoisomer- A |
| 117 | E11 | 1 | Bond | 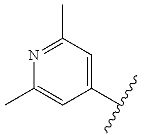 [SMILES: *c1cc(C)nc(C)c1] | CH₃ | b-9 (Q¹ = N, R^{3b} = CH₃) | 1′-RS*, 3-RS* Single diastereoisomer- B |
| 118 | E11 | 1 | CH₂ | 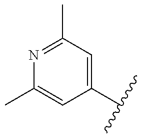 [SMILES: *c1cc(C)nc(C)c1] | CH₃ | b-9 (Q¹ = N, R^{3b} = CH₃) | 1′-R*, 3-R •2HCl |
| 119 | E11 | 1 | CH₂ | 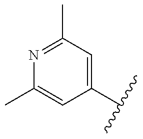 [SMILES: *c1cc(C)nc(C)c1] | CH₃ | b-9 (Q¹ = N, R^{3b} = CH₃) | 1′-S*, 3-R •2HCl |
| 120 | E31 | 1 | OCH₂ | 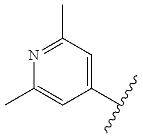 [SMILES: *c1cc(C)nc(C)c1] | H | b-1 (Q¹ = CH, Q² = NH, R^{1b} = H, R^{2b} = CH₃) | 3-RS •HCl |
| 121 | E11 | 1 | CH₂ | 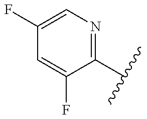 [SMILES: *c1ncc(F)cc1F] | H | b-1 (Q¹ = CH, Q² = S, R^{1b} = H, R^{2b} = CH₃) | 3-R |
| 122 | E11 | 1 | CH₂ | 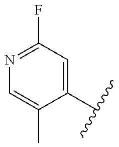 [SMILES: *c1cc(F)ncc1C] | H | b-1 (Q¹ = CH, Q² = S, R^{1b} = H, R^{2b} = CH₃) | 3-R |
| 123 | E11 | 1 | CH₂ | 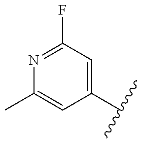 [SMILES: *c1cc(C)nc(F)c1] | H | b-1 (Q¹ = CH, Q² = S, R^{1b} = H, R^{2b} = CH₃) | 3-R |
| 124 | E11 | 1 | CH₂ | 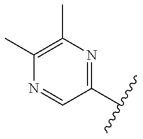 [SMILES: *c1cnc(C)c(C)n1] | H | b-1 (Q¹ = CH, Q² = S, R^{1b} = H , R^{2b} = CH₃) | 3-R |
| 125 | E11 | 1 | CH₂ | 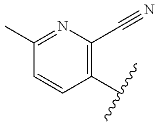 [SMILES: *c1ccc(C)nc1C#N] | H | b-1 (Q¹ = CH, Q² = S, R^{1b} = H, R^{2b} = CH₃) | 3-R |
| 126 | E20 | 1 | CH₂ | 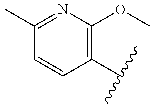 | H | b-1 (Q¹ = CH, Q² = S, R^{1b} = H, R^{2b} = CH₃) | 3-R |
| 127 | E20 | 1 | CH₂ | 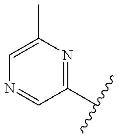 [SMILES: *c1cncc(C)n1] | H | b-1 (Q¹ = CH, Q² = S, R^{1b} = H, R^{2b} = CH₃) | 3-R |
| 128 | E20 | 1 | CH₂ | 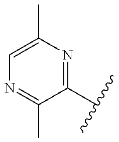 [SMILES: *c1nc(C)cnc1C] | H | b-1 (Q¹ = CH, Q² = S, R^{1b} = H, R^{2b} = CH₃) | 3-R |
| 129 | E20 | 1 | CH₂ | 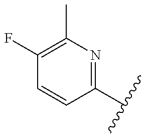 [SMILES: *c1ccc(F)c(C)n1] | H | b-1 (Q¹ = CH, Q² = S, R^{1b} = H, R^{2b} = CH₃) | 3-R |
| 130 | E27 | 1 | Bond | 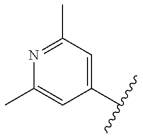 [SMILES: *c1cc(C)nc(C)c1] | H | b-1 (Q¹ = CH, Q² = S, R^{1b} = H, R^{2b} = CH₃) | 3-R* |
| 131 | E27 | 1 | Bond | 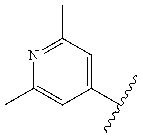 [SMILES: *c1cc(C)nc(C)c1] | H | b-1 (Q¹ = CH, Q² = S, R^{1b} = H, R^{2b} = CH₃) | 3-S* |
| 132 | E20 | 1 | OCH₂ | 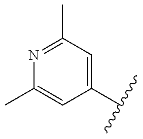 [SMILES: *c1cc(C)nc(C)c1] | H | b-1 (Q¹ = CH, Q² = NH, R^{1b} = H, R^{2b} = CH₃) | 3-RS •2HCl |
| 133 | E32 | 1 | OCH₂ | 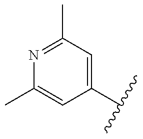 [SMILES: *c1cc(C)nc(C)c1] | CH₃ | b-1 (Q¹ = CH, Q² = S, R^{1b} = H, R^{2b} = CH₃) | 1′-RS, 3-RS |
| 134 | E32 | 1 | OCH₃ | 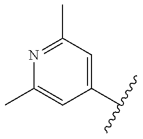 [SMILES: *c1cc(C)nc(C)c1] | CH₃ | b-1 (Q¹ = CH, Q² = S, R^{1b} = H, R^{2b} = CH₃) | 1′-R*, 3-S* |
| 135 | E32 | 1 | OCH₂ | 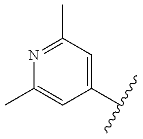 [SMILES: *c1cc(C)nc(C)c1] | CH₃ | b-1 (Q¹ = CH, Q² = S, R^{1b} = H, R^{2b} = CH₃) | 1′-S*, 3-S* |
| 136 | E32 | 1 | OCH₂ | 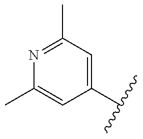 [SMILES: *c1cc(C)nc(C)c1] | CH₃ | b-1 (Q¹ = CH, Q² = S, R^{1b} = H , R^{2b} = CH₃) | 1′-S*, 3-R* |
| 137 | E32 | 1 | OCH₂ | 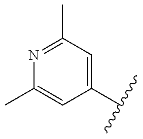 [SMILES: *c1cc(C)nc(C)c1] | CH₃ | b-1 (Q¹ = CH, Q² = S, R^{1b} = H, R^{2b} = CH₃) | 1′-R*, 3-R* |
| 138 | E33 | 1 | OCH₂ | 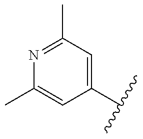 [SMILES: *c1cc(C)nc(C)c1] | H | b-1 (Q¹ = CH, Q² = O, R^{1b} = H, R^{2b} = CH₃) | 3-RS |
| 139 | E11 | 1 | CH₂ | 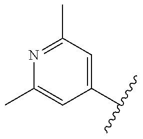 [SMILES: *c1cc(C)nc(C)c1] | CH₃ | b-4 | 1′-RS, 3-R |
| 140 | E20 | 1 | OCH₂ | 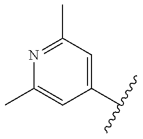 [SMILES: *c1cc(C)nc(C)c1] | H | b-1 (Q¹ = CH, Q² = S, R^{1b} = H, R^{2b} = CH₃) | 3-S |
| 141 | E11 | 1 | CH₂ | 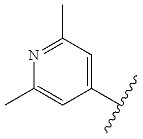 [SMILES: *c1cc(C)nc(C)c1] | CH₃ | b-2 | 1′-RS, 3-R |
| 142 | E11 | 1 | CH₂ | 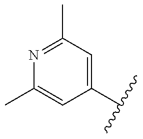 [SMILES: *c1cc(C)nc(C)c1] | CH₃ | b-3 | 1′-RS, 3-R •2HCl |
| 143 | E20 | 0 | CH₂ | 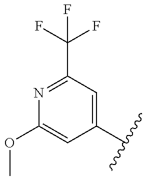 [SMILES: *c1cc(OC)nc(C(F)(F)F)c1] | H | b-1 (Q¹ = CH, Q² = S, R^{1b} = H, R^{2b} = CH₃) | 3-S |
| 144 | E11 | 1 | bond | 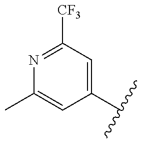 [SMILES: *c1cc(C)nc(C(F)(F)F)c1] | CH₃ | b-4 | 1′-RS, 3-RS |
| 145 | E20 | 1 | CH₂ | 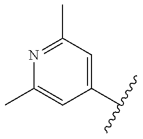 [SMILES: *c1cc(C)nc(C)c1] | H | b-11 (R^{4b} = CH₃) | 3-R •2HCl |
| 146 | E20 | 1 | CH₂ | 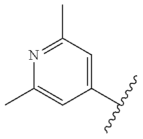 [SMILES: *c1cc(C)nc(C)c1] | H | b-2 | 3-R |
| 147 | E20 | 1 | CH₂ | 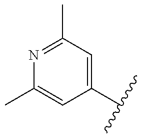 [SMILES: *c1cc(C)nc(C)c1] | H | b-11 (R^{4b} = H) | 3-R •2HCl |
| 148 | E11 | 1 | CH₂ | 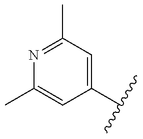 [SMILES: *c1cc(C)nc(C)c1] | CH₃ | b-11 (R^{4b} = H) | 1′-RS, 3-R •2HCl |
| 149 | E11 | 1 | CH₂ | 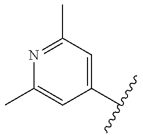 [SMILES: *c1cc(C)nc(C)c1] | CH₃ | b-9 (Q¹ = N, R^{3b} = H) | 1′-RS, 3-R |
| 150 | E20 | 1 | CH₂ | 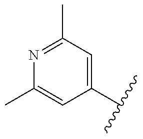 [SMILES: *c1cc(C)nc(C)c1] | H | b-9 (Q¹ = CH, R^{3b} = CH₃) | 3-R |
| 151 | E20 | 1 | CH₂ | 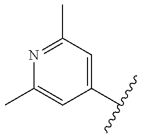 [SMILES: *c1cc(C)nc(C)c1] | H | b-9 (Q¹ = N, R^{3b} = H) | 3-R |
| 152 | E20 | 1 | CH₂ | 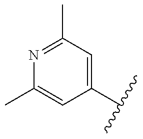 [SMILES: *c1cc(C)nc(C)c1] | H | b-3 | 3-R |
| 153 | E20 | 1 | CH₂ | 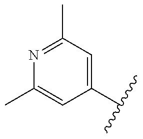 [SMILES: *c1cc(C)nc(C)c1] | H | b-9 (Q¹ = N, R^{3b} = CH₃) | 3-R |
| 154 | E20 | 1 | OCH₂ | 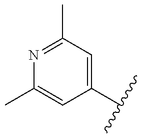 [SMILES: *c1cc(C)nc(C)c1] | H | b-1 (Q¹ = CH, Q² = S, R^{1b} = H, R^{2b} = CH₃) | 3-R |
| 155 | E20 | 1 | NH | 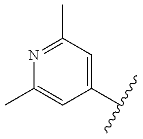 [SMILES: *c1cc(C)nc(C)c1] | H | b-1 (Q¹ = CH, Q² = S, R^{1b} = H, R^{2b} = CH₃) | 3-RS |
| 156 | E11 | 1 | NH | 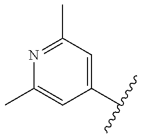 [SMILES: *c1cc(C)nc(C)c1] | CH₃ | b-4 | 1′-RS, 3-S |
| 172 | E36 | 1 | CH₂ | 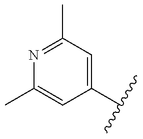 [SMILES: *c1cc(C)nc(C)c1] | H | b-1 (Q¹ = CH, Q² = NH, R^{1b} = H, R^{2b} = CH₃) | 3-R |
| 173 | E37 | 1 | CH₂ | 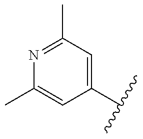 [SMILES: *c1cc(C)nc(C)c1] | CH₃ | b-1 (Q¹ = CH, Q² = NH, R^{1b} = H, R^{2b} = CH₃) | 1′-RS, 3-R |
| 174 | E38 | 1 | CH₂ | 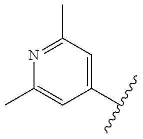 [SMILES: *c1cc(C)nc(C)c1] | H | b-1 (Q¹ = CH, Q² = NCH₃, R^{1b} = H, R^{2b} = CH₃) | 3-R |

^{#}means reference compound.

TABLE 3

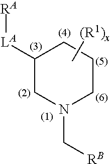

| Co. | Exp | | | | | | Stereochem/ |
|---|---|---|---|---|---|---|---|
| no. | no. | R¹ | x | L^{A} | R^{A} | R^{B} | Salt |
| 157 | E20 | 3-F | 1 | OCH₂ | 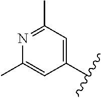 [SMILES: *c1cc(C)nc(C)c1] | b-4 | 3-RS |
| 158 | E20 | 5-CF₃ | 1 | OCH₂ | 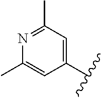 [SMILES: *c1cc(C)nc(C)c1] | b-1 (Q¹ = CH, Q² = S, R^{1b} = H, R^{2b} = CH₃) | 3-RS*, 5-RS* cis isomer |
| 159 | E20 | 3-CF₃ | 1 | OCH₂ | 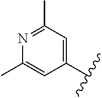 [SMILES: *c1cc(C)nc(C)c1] | b-1 (Q¹ = CH, Q² = S, R^{1b} = H, R^{2b} = CH₃) | 3-RS*, 5-RS* trans isomer |
| 160 | E20 | 3-F | 1 | OCH₂ | 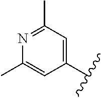 [SMILES: *c1cc(C)nc(C)c1] | b-1 (Q¹ = CH, Q² = S, R^{1b} = H, R^{2b} = CH₃) | 3-RS |
| 161 | E20 | 5-F | 2 | OCH₂ | 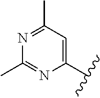 [SMILES: *c1cc(C)nc(C)n1] | b-1 (Q¹ = CH, Q² = S, R^{1b} = H, R^{2b} = CH₃) | 3-RS |
| 162 | E20 | 4-F | 2 | OCH₂ | 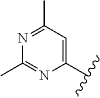 [SMILES: *c1cc(C)nc(C)n1] | b-1 (Q¹ = CH, Q² = S, R^{1b} = H, R^{2b} = CH₃) | 3-RS |
| 163 | E20 | 4-F | 1 | OCH₂ | 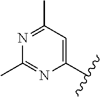 [SMILES: *c1cc(C)nc(C)n1] | b-1 (Q¹ = CH, Q² = S, R^{1b} = H, R^{2b} = CH₃) | 3-RS, 4-RS |
| 164 | E20 | 5-F | 1 | OCH₂ | 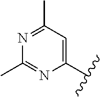 [SMILES: *c1cc(C)nc(C)n1] | b-1 (Q¹ = CH, Q² = S, R^{1b} = H, R^{2b} = CH₃) | 3-RS, 4-RS cis isomer |
| 165 | E20 | 6-CH₃ | 1 | CH₂ | 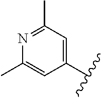 [SMILES: *c1cc(C)nc(C)c1] | b-1 (Q¹ = CH, Q² = S, R^{1b} = H, R^{2b} = CH₃) | 3-RS*, 6-RS* trans isomer |
| 166 | E20 | 6-CH₃ | 1 | CH₂ | 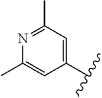 [SMILES: *c1cc(C)nc(C)c1] | b-1 (Q¹ = CH, Q² = S, R^{1b} = H, R^{2b} = CH₃) | 3-RS*, 6-RS* cis isomer |
| 167 | E34 | 6-CH₃ | 1 | CH₂ | 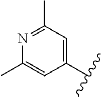 [SMILES: *c1cc(C)nc(C)c1] | b-1 (Q¹ = CH, Q² = S, R^{1b} = H, R^{2b} = CH₃) | 3-R*, 6-R* cis isomer |
| 168 | E34 | 6-CH₃ | 1 | CH₂ | 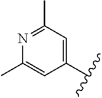 [SMILES: *c1cc(C)nc(C)c1] | b-1 (Q¹ = CH, Q² = S, R^{1b} = H, R^{2b} = CH₃) | 3-S*, 6-S* cis isomer |
| 169 | E20 | 2-CH₃ | 1 | CH₂ | 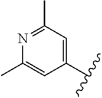 [SMILES: *c1cc(C)nc(C)c1] | b-1 (Q¹ = CH, Q² = S, R^{1b} = H, R^{2b} = CH₃) | 2-RS*, 3-RS* cis isomer |
| 170 | E20 | 2-CH₃ | 1 | CH₂ | 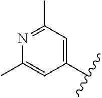 [SMILES: *c1cc(C)nc(C)c1] | b-1 (Q¹ = CH, Q² = S, R^{1b} = H, R^{2b} = CH₃) | 2-R*, 3-S* cis isomer |
| 171 | E20 | 2-CH₃ | 1 | CH₂ | 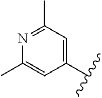 [SMILES: *c1cc(C)nc(C)c1] | b-1 (Q¹ = CH, Q² = S, R^{1b} = H, R^{2b} = CH₃) | 2-S*, 3-R* cis isomer |

### C. ANALYTICAL PART

Melting Points

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values (A).

Mettler Toledo Mettler FP 81HT/FP90 apparatus (B) or Mettler Toledo MP50 (C): For a number of compounds, melting points were determined in open capillary tubes on a Mettler FP 81HT/FP90 apparatus. Melting points were measured with a temperature gradient of 1, 3, 5 or 10° C./minute. Maximum temperature was 300° C. The melting point was read from a digital display.

LCMS

General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 4
LC-MS Methods (Flow expressed in mL/min; column
temperature (T) in ° C.; Run time in min).

| | | | | | Flow | Run |
|---|---|---|---|---|---|---|
| Method | Instrument | Column | Mobile Phase | Gradient | Col T | Time |
| 1 | Agilent | YMC-pack | A: 0.1% | From 95% A | 2.6 | 6.2 |
| | 1100 HPLC | ODS-AQ C18 (3 μm | HCOOH in | to 5% A in | 35 | |
| | DAD | 50 × 4.6 mm) | H₂O | 4.8 min, held | | |
| | LC/MS | | B: CH₃CN | for 1.0 min, | | |
| | G1956A | | | to 95% A in | | |
| | | | | 0.2 min | | |
| 2 | Waters: | Waters: BEH | A: 95% | From 95% A | 1 | 5 |
| | Acquity ® | C18 (1.7 μm, | CH₃COONH₄ | to 5% A in | 50 | |
| | UPLC ® - | 2.1 × 50 mm) | 6.5 mM + | 4.6 min, held | | |
| | DAD/SQD | | 5% CH₃CN, | for 0.4 min | | |
| | | | B: CH₃CN | | | |
| 3 | Waters: | Waters: BEH | A: 95% | From 95% A | 1 | 5 |
| | Acquity ® | C18 (1.7 μm, | CH₃COONH₄ | to 5% A in | 50 | |
| | IClass | 2.1 × 50 mm) | 6.5 mM + | 4.6 min, held | | |
| | UPLC ® - | | 5% CH₃CN, | for 0.4 min | | |
| | DAD/Xevo | | B: CH₃CN | | | |
| | G2-S QTOF | | | | | |
| 4 | Waters: | Waters: BEH | A: 95% | From 95% A | 1 | 5 |
| | Acquity ® | C18 (1.7 μm, | CH₃COONH₄ | to 5% A in | 50 | |
| | IClass | 2.1 × 50 mm) | 6.5 mM + | 4.6 min, held | | |
| | UPLC ® - | | 5% CH₃CN, | for 0.4 min | | |
| | DAD/SQD | | B: CH₃CN | | | |
| 5 | Waters: | Waters: HSS T3 | A: 95% | From 100% | 0.7 | 3.5 |
| | Acquity ® | column (1.8 μm, | CH₃COONH₄ | A to 95% A | 55 | |
| | UPLC ® - | 2.1 × 100 mm) | 10 mM + | in 2.1 min, to | | |
| | DAD/SQD | | 5% CH₃CN, | 95% A in | | |
| | | | B: CH₃CN | 0.9 min, held | | |
| | | | | for 0.5 min | | |
| 6 | Agilent: | Agilent: | A: CF₃COOH | 98% A for | 1 | |
| | 1100/1200 - | Eclipse ® | 0.1% in water, | 3 min, to | RT | 28 |
| | DAD and | C18 (5 μm, | B: CH₃CN | 100% B in | | |
| | MSD | 4.6 × 150 mm) | | 12 min, held | | |
| | | | | for 5 min, | | |
| | | | | back to 98% | | |
| | | | | A in 2 min, | | |
| | | | | held for | | |
| | | | | 6 min. | | |
| 7 | Agilent | Phenomenex | A: 50 mM | From 95% A | 0.7 | 6.2 |
| | 1100 HPLC | Kinetex | NH₄OAc in | to 5% A in | 35 | |
| | DAD | C18 (50 × | H₂O | 4.8 min, held | | |
| | LC/MS | 2.1 mm, 2.6 μm) | B: CH₃CN | for 1.0 min, | | |
| | G1956A | | | to 95% A in | | |
| | | | | 0.2 min. | | |
| 8 | Waters: | Agilent: | A: 95% | From 95% A | 1 | 5 |
| | Acquity ® | RRHD (1.8 μm, | CH₃COONH₄ | to 5% A in | 50 | |
| | IClass | 2.1 × 50 mm) | 6.5 mM + | 4.6 min, held | | |
| | UPLC ® - | | 5% CH₃CN, | for 0.4 min | | |
| | DAD and | | B: CH₃CN | | | |
| | SQD | | | | | |
| 9 | Waters: | Waters: HSS T3 | A: 95% | From 100% | 0.7 | 3.5 |
| | Acquity ® | column (1.8 μm, | CH₃COONH₄ | A to 95% A | 55 | |
| | UPLC ® - | 2.1 × 100 mm) | 10 mM + | in 2.1 min, to | | |
| | DAD/SQD | | 5% CH₃CN, | 95% A in | | |
| | | | B: CH₃CN | 0.9 min, held | | |
| | | | | for 0.5 min | | |
| 10 | Agilent: | Agilent: | A: 95% | From 95% A | 1 | 7 |
| | HP1100- | Eclipse Plus | CH₃COONH₄ | to 0% A in | 60 | |
| | DAD/MSD | C18 (3.5 μm, | 6.5 mM + | 5.0 min, held | | |
| | G1956B | 2.1 × 30 mm) | 5% CH₃CN, | for 0.15 min, | | |
| | | | B: CH₃CN | back to 95% | | |
| | | | | A in 0.15 min, | | |
| | | | | held for | | |
| | | | | 1.7 min | | |
| 11 | Agilent | YMC-pack ODS-AQ | A: 0.1% | 100% A held | 2.6 | 6.2 |
| | 1100 HPLC | C18 (50 × | HCOOH in | for 0.2. From | 35 | |
| | DAD | 4.6 mm, 3 μm) | H₂O | 100% A to | | |
| | LC/MS | | B: CH₃CN | 50% A in 4.5 | | |
| | G1956A | | | min, and to | | |
| | | | | 5% A in 0.1 | | |
| | | | | min, held for | | |
| | | | | 1.0 min, to | | |
| | | | | 95% A in 0.2 | | |
| | | | | min. | | |
| 12 | Waters: | Waters: HSS T3 | A: 95% | From 100% | 0.7 | 3.5 |
| | Acquity ® | column (1.8 μm, | CH₃COONH₄ | A to 95% A | 40 | |
| | UPLC ® - | 2.1 × 100 mm) | 10 mM + | in 2.1 min, to | | |
| | DAD/SQD | | 5% CH₃CN, | 95% A in | | |
| | | | B: CH₃CN | 0.9 min, held | | |
| | | | | for 0.5 min | | |
| 13 | Waters: | Waters: BEH | A: 95% | From 95% A | 1 | 2 |
| | Acquity ® | C18 (1.7 μm, | CH₃COONH₄ | to 40% A in | 50 | |
| | UPLC ® - | 2.1 × 50 mm) | 6.5 mM + | 1.2 min, to | | |
| | DAD/SQD | | 5% CH₃CN, | 5% A in | | |
| | | | B: CH₃CN | 0.6 min, held | | |
| | | | | for 0.2 min | | |
| 14 | Waters: | Waters: BEH | A: 95% | 84.2% A for | 0.343 | 6.2 |
| | Acquity | C18 (1.7 μm, | CH₃COONH₄ | 0.49 min, to | 40 | |
| | UPLC ®- | 2.1 × 100 mm) | 7 mM/ | 10.5% Ain | | |
| | DAD and | | 5% CH₃CN, | 2.18 min, held | | |
| | Quattro | | B: CH₃CN | for 1.94 min, | | |
| | Micro ™ | | | back to | | |
| | | | | 84.2% A in | | |
| | | | | 0.73 min, held | | |
| | | | | for 0.73 min. | | |
| 15 | Waters: | Waters: BEH | A: 95% | From 84.2% | 0.343 | 6.1 |
| | Acquity | C18 (1.7 μm, | CH₃COONH₄ | A to 10.5% A | 40 | |
| | UPLC ® | 2.1 × 100 mm) | 7 mM/ | in 2.18 min, | | |
| | H-Class - | | 5% CH₃CN, | held for | | |
| | DAD and | | B: CH₃CN | 1.94 min, | | |
| | SQD 2 | | | back to | | |
| | | | | 84.2% A in | | |
| | | | | 0.73 min, held | | |
| | | | | for 0.73 min. | | |

TABLE 5
Analytical data - melting point (M.p.) and LCMS: [M +
H]⁺ means the protonated mass of the free base of the compound,
[M − H]⁻ means the deprotonated mass of the free base of the compound
or the type of adduct specified [M + CH₃COO]⁻). Rₜ
means retention time (in min). For some compounds, exact mass
was determined.

| Co. No. | M.p. (° C.) | | [M + H]⁺ | Rₜ | LCMS Method |
|---|---|---|---|---|---|
| 1 | n.d. | | 411 | 1.42 | 3 |
| 2 | n.d. | | 397 | 1.37 | 3 |
| 3 | 226.08 | (A) | 367 | 1.13 | 3 |
| 4 | n.d. | | 367 | 1.26 | 3 |
| 5 | 155.3 | (A) | 316 | 9.3 | 6 |
| 6 | 209.1 | (C) | 331 | 0.37 | 7 |
| 7 | 178.2 | (C) | 345 | 1.41 | 1 |
| 8 | n.d. | | 360 | 0.86 | 3 |
| 9 | n.d. | | 360 | 0.89 | 3 |
| 10 | n.d. | | 348 | 1.14 | 3 |
| 11 | n.d. | | 362 | 1.36 | 3 |
| 12 | n.d. | | 348 | 1.17 | 3 |
| 13 | n.d. | | 360 | 2.56 | 3 |
| 14 | n.d. | | 360 | 1.31 | 3 |
| 15 | 146.1 | (A) | 361 | 1.53 | 3 |
| 16 | n.d. | | 361 | 1.56 | 3 |
| 17 | n.d. | | 347 | 1.52 | 3 |
| 18 | n.d. | | 349 | 1.69 | 3 |
| 19 | n.d. | | 363 (minor | 1.86/1.91 | 3 |
| | | | ion)/240 | | |
| | | | (fragment) | | |
| 20 | 128 | (C) | 375 | 0.96 | 1 |
| 21 | 213.1 | (C) | 375 | 1.45 | 1 |
| 22 | n.d. | | 346 | 0.71 | 3 |
| 23 | n.d. | | 346 | 0.71 | 3 |
| 24 | n.d. | | 360 | 1.02 | 2 |
| 25 | n.d. | | 360 | 1.31 | 5 |
| 26 | n.d. | | 360 | 1.30 | 5 |
| 27 | n.d. | | 359 | 1.72 | 3 |
| 28 | 153.1 | (A) | 359 | 1.54 | 5 |
| 29 | 150.5 | (A) | 359 | 1.55 | 5 |
| 30 | n.d. | | 348 | 0.86 | 3 |
| 31 | n.d. | | 360 | 0.85 | 1 |
| 32 | 94.5 | (C) | 374 | 1.67 | 1 |
| 33 | | | 409.1 | 1.71 | 3 |
| 34 | | | 399.1 | 1.66 | 3 |
| 35 | | | 399.1 | 1.66 | 3 |
| 36 | | | 399.1 | 1.63 | 3 |
| 37 | 113.97 | | 345.2 | 1.18 | 3 |
| 38 | 107.53 | | 345.2 | 1.17 | 3 |
| 39 | 150.08 | | 335.1 | 1.05 | 3 |
| 40 | | | 359.2 | 1.51 | 3 |
| 41 | | | 375.2 | 1.68 | 3 |
| 42 | | | 361.2 | 1.4 | 3 |
| 43 | 129.25 | | 349.1 | 1.23 | 3 |
| 44 | 133.56 | | 348.1 | 0.76 | 3 |
| 45 | 143.15 | | 342.1 | 0.89 | 3 |
| 46 | | | 342.1 | 0.9 | 3 |
| 47 | 158.97 | | 346.2 | 0.79 | 3 |
| 48 | 124.52 | | 365.1 | 1.16 | 3 |
| 49 | 124.67 | | 401.1 | 1.56 | 3 |
| 50 | 145.85 | | 367.1 | 1.09 | 3 |
| 51 | | | 399.1 | 1.56 | 3 |
| 52 | 144.16 | | 349.1 | 1.17 | 3 |
| 53 | 124.44 | | 385.1 | 1.45 | 3 |
| 54 | | | 399.1 | 1.66 | 3 |
| 55 | 137.85 | | 318.1 | 0.63 | 3 |
| 56 | 155.53 | | 335.1 | 1.04 | 3 |
| 57 | 145.2 | | 361.2 | 1.08 | 3 |
| 58 | | | 346.2 | 0.86 | 3 |
| 59 | 140.83 | | 332.2 | 0.69 | 3 |
| 60 | | | 399.1 | 1.66 | 3 |
| 61 | 119.64 | | 331.2 | 0.97 | 3 |
| 62 | 148.76 | | 332 | 0.69 | 3 |
| 63 | 98.38 | | 331.2 | 0.95 | 3 |
| 64 | 123.91 | | 386.1 | 1.34 | 3 |
| 65 | 185.24 | | 386.1 | 1.08 | 3 |
| 66 | 121.88 | | 348.1 | 1.03 | 3 |
| 67 | 117.40 | | 348.1 | 1.02 | 3 |
| 68 | 130.48 | | 385.1 | 1.46 | 3 |
| 69 | 161.57 | | 345.2 | 1.17 | 3 |
| 70 | 140.18 | | 345.2 | 1.07 | 3 |
| 71 | 141.44 | | 345.2 | 1.07 | 3 |
| 72 | 140.73 | | 345.2 | 1.09 | 3 |
| 73 | 139.31 | | 399.1 | 1.56 | 3 |
| 74 | 102.64 | | 331.2 | 0.96 | 3 |
| 75 | 111.65 | | 353.1 | 1.18 | 3 |
| 76 | 118.26 | | 399.1 | 1.63 | 3 |
| 77 | | | 346.1 | 0.78 | 3 |
| 78 | | | 335.2 | 1.37 | 3 |
| 79 | 132.57/158.7 | | 321.2 | 1.27 | 3 |
| 80 | | | 359.2 | 1.28 | 3 |
| 81 | 118.26 | | 361.2 | 1.11 | 3 |
| 82 | | | 352.1 | 1.49 | 3 |
| 83 | | | 338.1 | 1.34 | 3 |
| 84 | | | 356.1 | 1.18 | 3 |
| 85 | | | 345.2 | 1.19 | 3 |
| 86 | 140.80 | | 346.1 | 0.91 | 3 |
| 87 | | | 366.2 | 1.62/1.67 | 3 |
| 88 | | | 352.2 | 1.45/1.51 | 3 |
| 89 | 125.90 | | 361.1 | 1.05 | 3 |
| 90 | 157.42 | | 332.1 | 0.63 | 3 |
| 91 | 152.16 | | 343.1 | 0.91 | 3 |
| 92 | 160.26 | | 415.1 | 1.44 | 3 |
| 93 | | | 339.2 | 1.40/1.45 | 3 |
| 94 | | | 348.2 | 2.3 | 3 |
| 95 | | | 353.2 | 1.39/1.44 | 3 |
| 96 | | | 349.2 | 1.77 | 3 |
| 97 | 256.57 | | 335.2 | 1.68 | 3 |
| 98 | | | 334.2 | 2.32 | 3 |
| 99 | | | 325 | 2.5 | 10 |
| 100 | | | 347.2 | 1.43/1.49 | 3 |
| 101 | | | 339.2 | 1.26 | 3 |
| 102 | | | 346.1 | 0.88 | 3 |
| 103 | 147.16 | | 332.2 | 0.67 | 3 |
| 104 | | | 349.1 | 1.18 | 3 |
| 105 | | | 361.1 | 0.56 | 3 |
| 106 | 140.36 | | 349.2 | 1.23 | 3 |
| 107 | 173.68 | | 349.1 | 1.17 | 3 |
| 108 | 118.30 | | 332.1 | 0.71 | 3 |
| 109 | | | 361.2 | 1.35 | 3 |
| 110 | | | 349 | 1.22 | 3 |
| 111 | | | 349 | 1.22 | 3 |
| 112 | | | 345.2 | 1.06 | 3 |
| 113 | | | 345.2 | 1.06 | 3 |
| 114 | | | 340.2 | 1.09/1.14 | 3 |
| 115 | | | 346.2 | 0.81 | 3 |
| 116 | | | 366.2 | 2.12 | 3 |
| 117 | | | 366.2 | 2.19 | 3 |
| 118 | 270.1 | | 380.2 | 1.28 | 3 |
| 119 | | | 380.2 | 1.98 | 3 |
| 120 | 198.2 | | 358 | 0.58 | 1 |
| 121 | | | 367.1 | 1.52 | 3 |
| 122 | 156.64 | | 363.2 | 1.59 | 3 |
| 123 | 168.45 | | 363.2 | 1.59 | 3 |
| 124 | | | 360.2 | 1.11 | 3 |
| 125 | 164.89 | | 370.2 | 1.48 | 3 |
| 126 | | | 375.2 | 1.71 | 3 |
| 127 | | | 346.2 | 1.03 | 3 |
| 128 | 130.86 | | 360.2 | 1.13 | 3 |
| 129 | | | 363.2 | 1.46 | 3 |
| 130 | | | 345 | 1.48 | 12 |
| 131 | | | 345 | 1.48 | 12 |
| 132 | 198.2 | | 358 | 0.58 | 1 |
| 133 | | | 389.1 | 0.99 | 1 |
| 134 | | | 389 | 1.64 | 9 |
| 135 | | | 389 | 1.65 | 9 |
| 136 | | | 389 | 1.65 | 9 |
| 137 | | | 389 | 1.64 | 9 |
| 138 | | | 359.1 | 1.43 | 11 |
| 139 | | | 361.2 | 1.77 | 3 |
| 140 | | | 375 | 1.36 | 3 |
| 141 | | | 353.2 | 1.67/1.69 | 3 |
| 142 | 256.05 | | 367.2 | 1.61/1.63 | 3 |
| 143 | | | 415.1 | 2.04 | 3 |
| 144 | | | 401.2 | 2.53/2.57 | 3 |
| 145 | | | 366.2 | 1.86 | 3 |
| 146 | | | 339.2 | 1.65 | 3 |
| 147 | | | 352.2 | 1.72 | 3 |
| 148 | | | 366.2 | 1.72/1.76 | 3 |
| 149 | | | 349.2 | 1.64/1.65 | 3 |
| 150 | | | 348.2 | 2.83 | 3 |
| 151 | 285.54 | | 335.2 | 1.51 | 3 |
| 152 | | | 353.2 | 1.62 | 3 |
| 153 | 236.42 | | 349.2 | 2.1 | 8 |
| 154 | | | 375 | 1.34 | 3 |
| 155 | | | 346.1 | 0.41 | 1 |
| 156 | | | 362.2 | 1.24 | 3 |
| 158 | 208.2 | (C) | 443.1 | 1.425 | 11 |
| 159 | | | 443.2 | 3.076 | 11 |
| 160 | 163.1 | (C) | 393 | 0.85 | 1 |
| 161 | 154.8 | (C) | 412 | 1.791 | 1 |
| 162 | 154.7 | (C) | 412.0 | 2.596 | 11 |
| 163 | 158.0 | (C) | 394.2 | 1.194 | 1 |

| | | | | |
|---|---|---|---|---|
| 164 | | 394.2 | 1.256 | 1 |
| 165 | | 373.2 | 0.88 | 13 |
| 166 | | 373.21 | 1.69 | 3 |
| | | 371.19 | | |
| 167 | 139.57- | 373.2 | 2.52 | 14 |
| | 26.42 | 371.2 | | |
| | J/g (A)* | | | |
| 168 | 137.09- | 373.2 | 2.51 | 14 |
| | 22.71 | 371.2 | | |
| | J/g (A)* | | | |
| 169 | | 373.1 | 0.87 | 13 |
| 170 | | 373.5 | 2.26 | 15 |
| | | 371.5 | | |
| 171 | | 373.5 | 2.26 | 15 |
| | | 371.4 | | |
| 173 | | 356.2 | 0.86 | 3 |

n.d. means not determined;
*from 30 to 300° C. at 10° C./min 50 mL N₂

Optical Rotations

Optical rotations were measured on a Perkin-Elmer 341 polarimeter with a sodium lamp and reported as follows: [α]° (λ, c g/100 ml, solvent, T ° C.). [α]_{λ}^{T}=(100α)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation the concentration and solvent are always provided in parentheses after the rotation. The rotation is reported using degrees and no units of concentration are given (it is assumed to be g/100 mL).

TABLE 6
Optical Rotation data.

| | α_{D} | Wavelength | Concentration | | Temp. |
|---|---|---|---|---|---|
| Co. No. | (°) | (nm) | w/v % | Solvent | (° C.) |
| 8 | −61.4 | 589 | 0.84 | DMF | 20 |
| 9 | +60.4 | 589 | 0.65 | DMF | 20 |
| 10 | −40.4 | 589 | 0.54 | DMF | 20 |
| 12 | +49.0 | 589 | 0.49 | DMF | 20 |
| 15 | +7.7 | 589 | 0.55 | DMF | 20 |
| 16 | −7.5 | 589 | 0.57 | DMF | 20 |
| 22 | +27.7 | 589 | 0.50 | DMF | 20 |
| 23 | −29.4 | 589 | 0.5 | DMF | 20 |
| 35 | −5.7 | 589 | 0.48 | MeOH | 20 |
| 36 | −11.9 | 589 | 0.50 | MeOH | 20 |
| 37 | −18.1 | 589 | 0.66 | DMF | 20 |
| 38 | −11.4 | 589 | 0.59 | DMF | 20 |
| 39 | −4.7 | 589 | 0.60 | DMF | 20 |
| 40 | −8.0 | 589 | 0.50 | DMF | 20 |
| 41 | −20.1 | 589 | 0.53 | MeOH | 20 |
| 42 | −20.1 | 589 | 0.58 | MeOH | 20 |
| 43 | −1.5 | 589 | 0.67 | DMF | 20 |
| 45 | +1.5 | 589 | 0.50 | MeOH | 20 |
| 46 | −13.7 | 589 | 0.50 | MeOH | 20 |
| 47 | −10.5 | 589 | 0.45 | MeOH | 20 |
| 48 | −3.0 | 589 | 1.07 | MeOH | 20 |
| 49 | −7.6 | 589 | 0.55 | DMF | 20 |
| 50 | −10.5 | 589 | 0.54 | DMF | 20 |
| 51 | −25.0 | 589 | 0.50 | MeOH | 20 |
| 52 | −10.0 | 589 | 0.53 | DMF | 20 |
| 53 | −11.8 | 589 | 0.50 | DMF | 20 |
| 54 | −13.7 | 589 | 0.50 | MeOH | 20 |
| 55 | −15.2 | 589 | 0.50 | MeOH | 20 |
| 56 | −13.0 | 589 | 0.50 | MeOH | 20 |
| 57 | −16.7 | 589 | 0.50 | MeOH | 20 |
| 58 | −12.5 | 589 | 0.50 | MeOH | 20 |
| 59 | −10.7 | 589 | 0.65 | MeOH | 20 |
| 60 | −13.4 | 589 | 0.52 | DMF | 20 |
| 61 | −9.4 | 589 | 0.51 | DMF | 20 |
| 62 | −4.5 | 589 | 0.53 | DMF | 20 |
| 63 | −23.6 | 589 | 0.56 | MeOH | 20 |
| 64 | −2.8 | 589 | 0.61 | DMF | 20 |
| 65 | −1.7 | 589 | 0.67 | DMF | 20 |
| 66 | −23.1 | 589 | 0.62 | DMF | 20 |
| 67 | −6.4 | 589 | 0.55 | DMF | 20 |
| 68 | −4.3 | 589 | 0.56 | DMF | 20 |
| 69 | −10.5 | 589 | 0.69 | DMF | 20 |
| 71 | −19.6 | 589 | 0.66 | DMF | 20 |
| 72 | −19.9 | 589 | 0.53 | DMF | 20 |
| 73 | −15.4 | 589 | 0.64 | DMF | 20 |
| 74 | −16.0 | 589 | 0.56 | DMF | 20 |
| 75 | −1.4 | 589 | 0.62 | DMF | 20 |
| 78 | −31.6 | 589 | 0.49 | MeOH | 20 |
| 79 | −6.9 | 589 | 0.51 | MeOH | 20 |
| 80 | −13.7 | 589 | 0.58 | DMF | 20 |
| 82 | −0.4 | 589 | 0.50 | MeOH | 20 |
| 83 | −1.4 | 589 | 0.55 | MeOH | 20 |
| 87 | −1.8 | 589 | 0.50 | MeOH | 20 |
| 88 | −2.9 | 589 | 0.48 | MeOH | 20 |
| 91 | −3.2 | 589 | 0.71 | DMF | 20 |
| 92 | −1.3 | 589 | 0.66 | DMF | 20 |
| 93 | −1.6 | 589 | 0.80 | MeOH | 20 |
| 94 | −5.3 | 589 | 0.88 | MeOH | 20 |
| 95 | −3.2 | 589 | 0.67 | MeOH | 20 |
| 96 | −10.6 | 589 | 0.53 | MeOH | 20 |
| 98 | −1.7 | 589 | 0.57 | MeOH | 20 |
| 99 | −0.5 | 589 | 0.99 | MeOH | 20 |
| 100 | −0.5 | 589 | 0.60 | MeOH | 20 |
| 101 | +0.1 | 589 | 0.59 | MeOH | 20 |
| 110 | −12.3 | 589 | 0.52 | DMF | 20 |
| 111 | +13.5 | 589 | 0.52 | DMF | 20 |
| 112 | −18.5 | 589 | 0.56 | DMF | 20 |
| 113 | +20.1 | 589 | 0.58 | DMF | 20 |
| 118 | −2.1 | 589 | 0.71 | DMF | 20 |
| 119 | −37.6 | 589 | 0.87 | DMF | 20 |
| 121 | −6.8 | 589 | 0.53 | DMF | 20 |
| 122 | −15.6 | 589 | 0.59 | DMF | 20 |
| 123 | −13.1 | 589 | 0.61 | DMF | 20 |
| 124 | −11.0 | 589 | 0.28 | DMF | 20 |
| 125 | −8.8 | 589 | 0.32 | DMF | 20 |
| 126 | −13.4 | 589 | 0.67 | DMF | 20 |
| 127 | −5.1 | 589 | 0.61 | DMF | 20 |
| 128 | −3.3 | 589 | 0.83 | DMF | 20 |
| 129 | −4.9 | 589 | 0.83 | DMF | 20 |
| 130 | +77.9 | 589 | 0.99 | DMF | 20 |
| 131 | −64.2 | 589 | 0.99 | DMF | 20 |
| 139 | −18.5 | 589 | 0.52 | MeOH | 20 |
| 140 | +27.4 | 589 | 0.53 | DMF | 20 |
| 141 | −13.8 | 589 | 0.56 | MeOH | 20 |
| 142 | −16.8 | 589 | 0.57 | MeOH | 20 |
| 145 | −10.5 | 589 | 0.52 | MeOH | 20 |
| 146 | −7.8 | 589 | 0.53 | MeOH | 20 |
| 147 | −5.6 | 589 | 0.51 | MeOH | 20 |
| 148 | −18.0 | 589 | 0.52 | MeOH | 20 |
| 149 | −33.8 | 589 | 0.46 | MeOH | 20 |
| 150 | −25.0 | 589 | 0.52 | DMF | 20 |
| 151 | −20.7 | 589 | 0.53 | DMF | 20 |
| 152 | −14.7 | 589 | 0.56 | DMF | 20 |
| 153 | −11.0 | 589 | 0.59 | DMF | 20 |
| 154 | −15.4 | 589 | 0.52 | DMF | 20 |
| 167 | +33.1 | 589 | 0.84 | DMF | 20 |
| 168 | −30.0 | 589 | 1.03 | DMF | 20 |
| 170 | +18.1 | 589 | 0.5 | DMF | 20 |
| 171 | −24.7 | 589 | 0.52 | DMF | 20 |
| 174 | −6.4 | 589 | 0.32 | DMF | 20 |

SFCMS-Methods

General Procedure for SFC-MS Methods

The SFC measurement was performed using Analytical Supercritical fluid chromatography (SFC) system composed by a binary pump for delivering carbon dioxide (CO2) and modifier, an autosampler, a columns oven with switching valve for column heating from room temperature to 80° C., a diode array detector equipped with a high-pressure flow cell standing up to 400 bars. Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 7
Analytical SFC-MS Methods (Flow expressed in mL/min;
column temperature (T) in ° C.; Backpressure in bars).

| | | | | Flow | Run time |
|---|---|---|---|---|---|
| Method | Column | Mobile Phase | Gradient | T | BPR |
| 1 | Daicel Chiralpak ® | A: CO₂ | 10%-50% B | 2.5 | 9.5 |
| | IC3 column (3.0 μm, | B: EtOH + 0.2% | in 6 min, | 40 | 110 |
| | 150 × 4.6 mm) | iPrNH₂ | hold 3.5 min | | |
| 2 | Daicel Chiralpak ® | A: CO₂ | 10%-50% B | 2.5 | 9.5 |
| | AD3 column (3.0 μm, | B: iPOH | in 6 min, | 40 | 110 |
| | 150 × 4.6 mm) | (+0.2% iPrNH₂) | hold 3.5 min | | |
| 3 | Daicel Chiralpak ® | A: CO₂ | 10%-50% B | 2.5 | 9.5 |
| | AD3 (150 × 4.6 mm, | B: iPrOH + 0.2% | in 6 min, | 40 | 130 |
| | 3 μm) | iPrNH₂ | hold 3.5 min | | |
| 4 | Daicel Chiralpak ® | A: CO₂ | 10%-50% B | 2.5 | 9.5 |
| | AD3 (150 × 4.6 mm, | B: MeOH | in 6 min, | 40 | 130 |
| | 3 μm) | (+0.2% iPrNH₂) | hold 3.5 min | | |
| 5 | Daicel Chiralpak | A: CO₂ | 40% B hold 3 | 3.5 | 3.0 |
| | AD-3 (100 × 4.6 mm, | B: MeOH | min | 35 | 105 |
| | 3 μm) | (+0.3% iPrNH₂) | | | |
| | | 60/40 | | | |
| 6 | Daicel Chiralpak | A: CO2 | 30% B hold 3 | 3.5 | 3.0 |
| | AD-3 (100 × 4.6 mm, | B: iPrOH | min | 35 | 105 |
| | 3 μm) | (+0.3% iPrNH2) | | | |
| | | 70/30 | | | |
| 7 | Daicel Chiralpak ® | A: CO₂ | 45% B hold | 3.5 | 3 |
| | IC-3 (3 μm, | B: EtOH(0.3% | 3 min, | 35 | 105 |
| | 100 × 4.6 mm) | iPrNH₂) | | | |

TABLE 8
Analytical SFC data - Rₜ means retention time (in
minutes), [M + H]⁺ means the protonated
mass of the compound, method refers to the method
used for (SFC)MS analysis of enantiomerically pure compounds.

| | | | | | Isomer Elution |
|---|---|---|---|---|---|
| Co. No. | Rₜ | [M + H]⁺ | UV Area % | Method | Order |
| 25 | 5.87 | 360 | 100 | 2 | A |
| 26 | 6.30 | 360 | 100 | 2 | B |
| 28 | 5.47 | 359 | 100 | 1 | A |
| 29 | 6.14 | 359 | 99.3 | 1 | B |
| 69 | 1.62 | 344 | 100 | 5 | B |
| 70 | 1.14 | 344 | 100 | 5 | A |
| 130 | 1.03 | 344 | 100 | 6 | A |
| 131 | 1.18 | 344 | 100 | 6 | B |
| 134 | 4.38 | 389 | 94.25 | 4 | C |
| 135 | 4.61 | 389 | 100 | 4 | D |
| 136 | 4.41 | 389 | 100 | 3 | A |
| 137 | 4.61 | 389 | 96.07 | 3 | B |
| 167 | 1.08 | 373 | 97.12 | 6 | A |
| 168 | 1.5 | 373 | 100 | 6 | B |
| 170 | 1.24 | 373 | 100 | 7 | A |
| 171 | 1.76 | 373 | 100 | 7 | B |

(*) sample contains 2.88% of Co. No. 168

NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d6 sulfoxide) as solvent. Chemical shifts (6) are reported in parts per million (ppm) relative to tetramethylsilane (TMS), which was used as internal standard.

TABLE 9
¹H NMR results

| Co. No. | ¹H NMR result |
|---|---|
| 1 | ¹H NMR (500 MHz, DMSO-d6) δ ppm 1.63 (br d, J = 5.8 Hz, 2 H), 1.73-1.87 (m, |
| | 2 H), 2.21 (s, 3 H), 2.35 (s, 6 H), 2.95-3.03 (m, 1 H), 3.07-3.24 (m, 3 H), 4.69 |
| | (br s, 1 H), 6.67 (s, 2 H), 7.99 (s, 1 H), 12.75 (s, 1 H) |
| 2 | ¹H NMR (500 MHz, DMSO-d6) δ ppm 1.64 (br d, J = 5.8 Hz, 2 H), 1.82 (br dd, |
| | J = 7.1, 4.5 Hz, 2 H), 2.21 (s, 3 H), 2.41 (s, 3 H), 2.94-3.03 (m, 1 H), 3.07-3.14 |
| | (m, 1 H), 3.14-3.25 (m, 2 H), 4.73 (br s, 1 H), 6.83 (dd, J = 5.9, 2.5 Hz, 1 H), 6.89 |
| | (d, J = 2.3 Hz, 1 H), 7.99 (s, 1 H), 8.26 (d, J = 5.8 Hz, 1 H), 12.76 (s, 1 H) |
| 3 | ¹H NMR (500 MHz, DMSO-d6) δ ppm 1.87 (dq, J = 12.6, 8.6 Hz, 1 H), 2.18-2.26 |
| | (m, 4 H), 2.40 (s, 3 H), 3.17 (dd, J = 9.8, 8.7 Hz, 1 H), 3.22-3.29 (m, 1 H), 3.46 |
| | (ddd, J = 10.1, 8.1, 3.8 Hz, 2 H), 3.71 (dd, J = 9.8, 7.5 Hz, 1 H), 7.03 (d, J = 4.9 Hz, 1 |
| | H), 7.08 (s, 1 H), 8.04 (s, 1 H), 8.31 (d, J = 5.2 Hz, 1 H), 12.69 (br s, 1 H) |
| 4 | ¹H NMR (400 MHz, DMSO-d6) δ ppm 1.74-1.87 (m, 1 H), 2.07 (s, 3 H), 2.13- |
| | 2.22 (m, 1 H), 2.41 (s, 3 H), 3.04-3.11 (m, 1 H), 3.16-3.33 (m, 3 H), 3.40-3.47 |
| | (m, 1 H), 3.64 (dd, J = 9.8, 7.5 Hz, 1 H), 7.15 (d, J = 7.9 Hz, 1 H), 7.50 (dd, J = 8.1, |
| | 2.5 Hz, 1 H), 7.86 (s, 1 H), 8.28 (d, J = 2.3 Hz, 1 H) |
| 6 | ¹H NMR (300 MHz, DMSO-d6) δ ppm 1.34-1.63 (m, 2 H) 1.63-1.85 (m, 2 H) |
| | 1.94-2.10 (m, 2 H) 2.11 (s, 3 H) 2.41 (s, 3 H) 2.65-2.78 (m, 1 H) 2.84 (br d, |
| | J = 11.0 Hz, 2 H) 3.66 (s, 2 H) 7.05 (br d, J = 4.9 Hz, 1 H) 7.13 (s, 1 H) 7.24 (s, 1 H) |
| | 8.30 (d, J = 5.1 Hz, 1 H) 11.91 (br s, 1 H). |
| 7 | ¹H NMR (300 MHz, CDCl₃) δ ppm 1.41 (qd, J = 11.9, 4.2 Hz, 1 H), 1.59-1.99 (m, |
| | 3 H), 2.07 (br t, J = 10.7 Hz, 2 H), 2.31 (s, 3 H), 2.48 (s, 6 H), 2.74 (br t, J = 11.1 Hz, |
| | 1 H), 2.95 (br d, J = 10.4 Hz, 2 H), 3.63-3.79 (m, 2 H), 6.80 (s, 2 H), 7.19 (s, 1 H), |
| | 12.12 (br s, 1 H) |
| 8 | ¹H NMR (500 MHz, CDCl₃) δ ppm 1.52-1.69 (m, 3 H), 1.69-1.80 (m, 1 H), |
| | 2.25-2.35 (m, 1 H), 2.32 (s, 3 H), 2.36 (s, 6 H), 2.37-2.45 (m, 1 H), 2.55 (br s, 1 |
| | H), 2.60-2.69 (m, 1 H), 3.58-3.66 (m, 1 H), 3.68 (d, J = 2.0 Hz, 2 H), 4.58 (br s, 1 |
| | H), 6.16 (s, 2 H), 7.18 (s, 1 H), 12.41 (br s, 1 H) |
| 9 | ¹H NMR (400 MHz, CDCl₃) δ ppm 1.49-1.68 (m, 3 H), 1.68-1.80 (m, 1 H), |
| | 2.32 (s, 3 H), 2.34-2.47 (m, 2 H), 2.36 (s, 6 H), 2.50-2.69 (m, 2 H), 3.57-3.66 |
| | (m, 1 H), 3.64-3.73 (m, 2 H), 4.54 (br s, 1 H), 6.16 (s, 2 H), 7.18 (s, 1 H), 12.41 |
| | (br s, 1 H) |
| 10 | ¹H NMR (500 MHz, CDCl₃) δ ppm 1.60 (br s, 2 H), 1.65-1.83 (m, 2 H), 2.04 (br |
| | s, 1 H), 2.34 (s, 6 H), 2.37-2.58 (m, 2 H), 2.73 (br d, J = 6.9 Hz, 1 H), 3.64 (br s, 1 |
| | H), 3.70-3.80 (m, 2 H), 4.48 (br s, 1 H), 6.14 (s, 2 H), 7.83 (dd, J = 8.7, 1.4 Hz, 1 |
| | H), 8.02 (s, 1 H), 8.10 (d, J = 8.7 Hz, 1 H), 8.80-8.88 (m, 2 H) |
| 11 | ¹H NMR (400 MHz, CDCl₃) δ ppm 1.44-1.51 (m, 3 H), 1.52-1.81 (m, 4 H), |
| | 2.36 (s, 3.90 H), 2.38 (s, 2.10 H), 2.46-2.74 (m, 4 H), 3.52-3.63 (m, 1 H), 3.72- |
| | 3.85 (m, 1 H), 4.79 (br s, 1 H), 6.10 (s, 1.30 H), 6.13 (s, 0.70 H), 7.82-7.89 (m, 1 |
| | H), 7.97-8.02 (m, 1 H), 8.10 (d, J = 8.8 Hz, 0.35 H), 8.11 (d, J = 8.8 Hz, 0.65 H), |
| | 8.82-8.86 (m, 2 H). Mixture of diastereoisomers 65:35 |
| 12 | ¹H NMR (400 MHz, CDCl₃) δ ppm 1.51-1.82 (m, 4 H), 2.26 (br s, 1 H), 2.35 (s, |
| | 6 H), 2.37-2.48 (m, 1 H), 2.52 (br s, 1 H), 2.73 (br d, J = 9.5 Hz, 1 H), 3.59-3.69 |
| | (m, 1 H), 3.70-3.80 (m, 2 H), 4.51 (br s, 1 H), 6.14 (s, 2 H), 7.83 (dd, J = 8.6, 1.8 |
| | Hz, 1 H), 8.02 (d, J = 0.9 Hz, 1 H), 8.10 (d, J = 8.6 Hz, 1 H), 8.81-8.87 (m, 2 H) |
| 13 | ¹H NMR (500 MHz, CHLOROFORM-d) δ ppm 1.36-1.47 (m, 1 H), 1.57-1.70 |
| | (m, 1 H), 1.76-1.84 (m, 1 H), 2.03-2.16 (m, 3 H), 2.25 (s, 6 H), 2.30 (s, 3 H), |
| | 2.75-2.83 (m, 1 H), 3.12 (br dd, J = 10.7, 3.5 Hz, 1 H), 3.69-3.79 (m, 2 H), 4.27- |
| | 4.37 (m, 1 H), 6.53 (s, 2 H), 6.56 (s, 1 H), 7.18 (s, 1 H) |
| 14 | ¹H NMR (400 MHz, CDCl₃) δ ppm 1.38-1.51 (m, 1 H), 1.58-1.73 (m, 1 H), |
| | 1.77-1.87 (m, 1 H), 2.02-2.10 (m, 1 H), 2.10-2.22 (m, 2 H), 2.31 (s, 3 H), 2.43 |
| | (s, 6 H), 2.75-2.84 (m, 1 H), 3.06 (br dd, J = 10.6, 3.5 Hz, 1 H), 3.67-3.82 (m, 2 |
| | H), 4.36-4.46 (m, 1 H), 6.48 (s, 2 H), 7.19 (s, 1 H), 12.27 (br s, 1 H) |
| 15 | ¹H NMR (400 MHz, CDCl₃) δ ppm 1.79-1.90 (m, 1 H), 1.99-2.09 (m, 1 H), |
| | 2.16-2.27 (m, 3 H), 2.28 (s, 3 H), 2.47-2.54 (m, 1 H), 2.52 (s, 6 H), 2.72-2.81 |
| | (m, 1 H), 2.95-3.03 (m, 1 H), 3.67-3.77 (m, 2 H), 4.40-4.49 (m, 1 H), 6.53 (s, 2 |
| | H), 7.17 (s, 1 H), 9.87 (br s, 1 H) |
| 16 | 1H NMR (500 MHz, DMSO-d6) δ ppm 1.54-1.68 (m, 0.45 H), 1.74-1.86 (m, 1 |
| | H), 1.86-2.07 (m, 2 H), 2.16 (s, 3 H), 2.21 (br s, 0.55 H), 2.62 (s, 6 H), 2.80- |
| | 3.06 (m, 1.55 H), 3.25-3.61 (m, 2.45 H), 4.53 (br s, 2 H), 5.15 (br s, 0.55 H), 5.23 |
| | (br s, 0.45 H), 7.33 (br s, 1.10 H), 7.42 (br s, 0.90 H), 7.66 (br s, 1 H), 10.39- |
| | 10.91 (m, 0.55 H), 11.73 (br s, 0.45 H), 12.30 (br s, 1 H), 15.07 (br s, 1 H). |
| | Mixture of conformers 55:45 |
| 17 | ¹H NMR (400 MHz, CDCl₃) δ ppm 1.40-1.53 (m, 1 H), 1.59-1.73 (m, 1 H), |
| | 1.78-1.90 (m, 2 H), 2.01-2.12 (m, 1 H), 2.13-2.23 (m, 2 H), 2.30 (s, 3 H), 2.47 |
| | (s, 3 H), 2.74-2.83 (m, 1 H), 3.06 (br dd, J = 10.6, 3.7 Hz, 1 H), 3.69-3.80 (m, 1 |
| | H), 4.43 (tt, J = 9.0, 4.2 Hz, 1 H), 6.61 (dd, J = 5.8, 2.5 Hz, 1 H), 6.65 (d, J = 2.3 Hz, 1 |
| | H), 7.19 (s, 1 H), 8.26 (d, J = 6.0 Hz, 1 H), 11.87 (br s, 1 H) |
| 18 | ¹H NMR (400 MHz, CDCl₃) δ ppm 1.42-1.55 (m, 1 H), 1.62-1.76 (m, 1 H), |
| | 1.79-1.89 (m, 1 H), 2.04-2.14 (m, 1 H), 2.17-2.28 (m, 2 H), 2.40 (s, 6 H), 2.75- |
| | 2.83 (m, 1 H), 3.01-3.10 (m, 1 H), 3.73-3.87 (m, 2 H), 4.40-4.49 (m, 1 H), |
| | 6.45 (s, 2 H), 7.83 (dd, J = 8.6, 1.8 Hz, 1 H), 8.03 (d, J = 1.2 Hz, 1 H), 8.08 (d, J = 8.6 |
| | Hz, 1 H), 8.81-8.85 (m, 2 H) |
| 19 | ¹H NMR (500 MHz, CDCl₃) δ ppm 1.36-1.47 (m, 1 H), 1.48 (d, J = 6.9 Hz, 1.35 |
| | H), 1.48 (d, J = 6.9 Hz, 1.65 H), 1.59-1.73 (m, 1 H), 1.75-1.90 (m, 1 H), 2.02- |
| | 2.30 (m, 3 H), 2.34 (s, 3.30 H), 2.37 (s, 2.70 H), 2.71-2.77 (m, 0.45 H), 2.92- |
| | 2.98 (m, 0.55 H), 3.00-3.11 (m, 1 H), 3.78 (q, J = 6.6 Hz, 0.55 H), 3.86 (q, J = 6.8 |
| | Hz, 0.45 H), 4.30-4.43 (m, 1 H), 6.36 (s, 1.1 H), 6.41 (s, 0.90 H), 7.87-7.91 (m, |
| | 1 H), 8.00-8.02 (m, 1 H), 8.08 (d, J = 8.7 Hz, 0.45 H), 8.08 (d, J = 8.7 Hz, 0.55 H), |
| | 8.78-8.87 (m, 2 H). mixture 55:45 of diastereoisomers |
| 20 | ¹H NMR (300 MHz, CDCl₃) δ ppm 1.11 (br d, J = 9.6 Hz, 1 H), 1.55 (br d, J = 10.2 |
| | Hz, 3 H), 1.91-2.14 (m, 3 H), 2.22 (s, 3 H), 2.40 (s, 6 H), 2.68 (br d, J = 10.4 Hz, 1 |
| | H), 2.82 (br d, J = 9.5 Hz, 1 H), 3.51-3.69 (m, 2 H), 3.78 (br d, J = 6.0 Hz, 2 H), |
| | 6.41 (s, 2 H), 7.12 (s, 1 H), 11.30 (br s, 1 H) |
| 21 | 1H NMR (300 MHz, CDCl₃) δ ppm 1.31-1.49 (m, 1 H), 1.55-1.76 (m, 1 H), |
| | 1.77-1.93 (m, 1 H), 1.95-2.07 (m, 1 H), 2.33 (s, 5 H), 2.68 (s, 6 H), 2.80 (br s, 1 |
| | H), 3.02 (br d, J = 9.8 Hz, 1 H), 3.56-3.69 (m, 1 H), 3.85 (br s, 2 H), 4.47-4.63 |
| | (m, 2 H), 7.10 (s, 2 H), 7.29 (s, 1 H), 11.33 (br s, 1 H) |
| 22 | ¹H NMR (400 MHz, CDCl₃) δ ppm 1.64-1.75 (m, 1 H), 2.27-2.38 (m, 4 H), |
| | 2.40 (s, 6 H), 2.42-2.50 (m, 1 H), 2.66 (dd, J = 9.5, 3.0 Hz, 1 H), 2.73 (dd, J = 9.7, |
| | 6.2 Hz, 1 H), 2.92 (td, J = 8.6, 4.5 Hz, 1 H), 3.76-3.88 (m, 2 H), 3.99-4.09 (m, 1 |
| | H), 4.31 (br d, J = 7.9 Hz, 1 H), 6.14 (s, 2 H), 7.23 (s, 1 H), 11.93 (br s, 1 H) |
| 23 | ¹H NMR (400 MHz, CDCl₃) δ ppm 1.64-1.75 (m, 1 H), 2.27-2.37 (m, 4 H), |
| | 2.39 (s, 6 H), 2.42-2.50 (m, 1 H), 2.65 (dd, J = 9.5, 3.0 Hz, 1 H), 2.73 (dd, J = 9.5, |
| | 6.0 Hz, 1 H), 2.91 (td, J = 8.6, 4.5 Hz, 1 H), 3.74-3.89 (m, 2 H), 3.98-4.10 (m, 1 |
| | H), 4.34 (br d, J = 7.6 Hz, 1 H), 6.14 (s, 2 H), 7.23 (s, 1 H), 12.30 (br s, 1 H) |
| 24 | ¹H NMR (500 MHz, CDCl₃) δ ppm 1.02 (br d, J = 9.8 Hz, 1 H), 1.49-1.58 (m, 1 |
| | H), 1.62-1.69 (m, 2 H), 1.86 (br t, J = 10.0 Hz, 1 H), 2.02-2.14 (m, 2 H), 2.30 (s, |
| | 3 H), 2.43 (br s, 3 H), 2.50-2.61 (m, 2 H), 2.64 (br s, 3 H), 2.70-2.79 (m, 2 H), |
| | 3.57-3.67 (m, 2 H), 6.80 (s, 1 H), 7.16 (s, 1 H), 12.34 (br s, 1 H) |
| 25 | ¹H NMR (400 MHz, CDCl₃) δ ppm 0.96-1.11 (m, 1 H), 1.46-1.61 (m, 1 H), |
| | 1.62-1.70 (m, 2 H), 1.87 (br t, J = 10.2 Hz, 1 H), 2.02-2.18 (m, 2 H), 2.30 (s, 3 |
| | H), 2.43 (s, 3 H), 2.51-2.64 (m, 2 H), 2.65 (s, 3 H), 2.70-2.80 (m, 2 H), 3.57- |
| | 3.69 (m, 2 H), 6.80 (s, 1 H), 7.17 (s, 1 H), 11.94 (br s, 1 H) |
| 26 | ¹H NMR (400 MHz, CDCl₃) δ ppm 0.96-1.11 (m, 1 H), 1.48-1.61 (m, 1 H), |
| | 1.62-1.70 (m, 2 H), 1.87 (br t, J = 10.2 Hz, 1 H), 2.02-2.17 (m, 2 H), 2.31 (s, 3 |
| | H), 2.44 (s, 3 H), 2.51-2.64 (m, 2 H), 2.65 (s, 3 H), 2.71-2.80 (m, 2 H), 3.56- |
| | 3.69 (m, 2 H), 6.80 (s, 1 H), 7.17 (s, 1 H), 11.99 (br s, 1 H) |
| 27 | ¹H NMR (500 MHz, CDCl₃) δ ppm 0.90-1.01 (m, 1 H), 1.47-1.58 (m, 1 H), |
| | 1.61-1.70 (m, 2 H), 1.77-1.85 (m, 1 H), 1.86-1.95 (m, 1 H), 2.04 (br t, J = 10.4 |
| | Hz, 1 H), 2.31 (s, 3 H), 2.35-2.41 (m, 1 H), 2.43-2.49 (m, 1 H), 2.48 (s, 6 H), |
| | 2.74 (br d, J = 10.4 Hz, 1 H), 2.78 (br d, J = 10.4 Hz, 1 H), 3.58-3.71 (m, 2 H), 6.75 |
| | (s, 2 H), 7.17 (s, 1 H), 12.28 (br s, 1 H) |
| 28 | ¹H NMR (400 MHz, CDCl₃) δ ppm 0.88-1.02 (m, 1 H), 1.45-1.59 (m, 1 H), |
| | 1.60-1.72 (m, 2 H), 1.76-1.84 (m, 1 H), 1.84-1.96 (m, 1 H), 2.03 (br t, J = 10.2 |
| | Hz, 1 H), 2.31 (s, 3 H), 2.35-2.48 (m, 2 H), 2.47 (s, 6 H), 2.69-2.82 (m, 2 H), |
| | 3.57-3.70 (m, 2 H), 6.74 (s, 2 H), 7.16 (s, 1 H), 12.25 (s, 1 H) |
| 29 | ¹H NMR (400 MHz, CDCl₃) δ ppm 0.88-1.02 (m, 1 H), 1.45-1.59 (m, 1 H), |
| | 1.60-1.72 (m, 2 H), 1.76-1.84 (m, 1 H), 1.84-1.96 (m, 1 H), 2.03 (br t, J = 10.3 |
| | Hz, 1 H), 2.31 (s, 3 H), 2.34-2.49 (m, 2 H), 2.47 (s, 6 H), 2.69-2.82 (m, 2 H), |
| | 3.55-3.70 (m, 2 H), 6.74 (s, 2 H), 7.17 (s, 1 H), 12.40 (s, 1 H) |
| 30 | ¹H NMR (400 MHz, CDCl₃) δ ppm 1.43-1.54 (m, 1 H), 1.92-2.03 (m, 1 H), |
| | 2.25 (dd, J = 9.0, 6.2 Hz, 1 H), 2.31 (s, 3 H), 2.42-2.55 (m, 7 H), 2.56-2.71 (m, 5 |
| | H), 3.69-3.83 (m, 2 H), 6.76 (s, 2 H), 7.19 (s, 1 H), 12.39 (br s, 1 H) |
| 31 | ¹H NMR (300 MHz, DMSO-d6) δ ppm 1.09-1.25 (m, 1 H), 1.45-1.88 (m, 3 H), |
| | 1.95-2.05 (m, 2 H), 2.08 (s, 9 H), 2.72 (br d, J = 9.6 Hz, 1 H), 2.88 (br d, J = 10.0 |
| | Hz, 1 H), 3.33-3.41 (m, 1 H), 3.55-3.74 (m, 2 H), 6.04 (br d, J = 8.1 Hz, 1 H), |
| | 6.12 (s, 2 H), 7.18 (s, 1 H); |

### D. PHARMACOLOGICAL EXAMPLES

1) OGA—Biochemical Assay

The assay is based on the inhibition of the hydrolysis of fluorescein mono-3-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat # M1485) results in the formation of ß-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OriGene (cat # TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄2H₂O (Sigma, # C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg

FM-GlcNAc were dissolved in 5.48 mL DMSO to obtain a 250 mM solution and was stored at −20° C. OGA was used at a 10 nM (protocol A) or 2 nM (protocol B) concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 1 h (10 nM OGA, protocol A) or 6 h (2 nM OGA, protocol B). To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting O-GlcNAcylated residues as previoulsy described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, # D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, # D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, # A16046), pH 7.5 (ICC buffer) before being permeabilized in same buffer for 10 minutes. Samples are subsequently blocked in ICC containing 5% goat serum (Sigma, # G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, # A-21042) and nuclei stained with Hoechst 33342 at a final concentration of 1l g/ml in ICC (Lifetechnologies, # H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 10
Results in the biochemical and cellular assays.

| | | | Enzymatic | | Cellular |
|---|---|---|---|---|---|
| Co. | Enzymatic | Enzymatic | Emax | Cellular | Emax |
| no. | protocol | hOGA; pIC₅₀ | (%) | hOGA; pEC₅₀ | (%) |
| 1 | A | 6.18 | 96.3 | | |
| 2 | A | 5.98 | 89 | | |
| 3 | A | <5 | 15 | | |
| | B | <5 | 43 | | |
| 4 | B | 5 | 50.5 | | |
| 5 | A | 6.85 | 101.8 | | |
| 6 | B | 7.56 | 101.4 | 7.56 | 98.7 |
| 7 | B | 7.75 | 99.3 | 8.05 | 98.9 |
| 8 | B | 7.30 | 100.6 | | |
| 9 | B | 8.43 | 102.6 | 7.32 | 101.6 |
| 10 | B | 5.78 | 85.6 | | |
| 11 | B | 7.59 | 100.7 | 5.37 | 65.2 |
| 12 | B | 6.17 | 92.9 | | |
| 13 | B | 7.03 | 98.7 | 6.5 | 92.1 |
| 14 | A | 8.09 | 100.2 | 6.95 | 118.3 |
| | B | 8.04 | 101.3 | | |
| 15 | B | 8.07 | 102.1 | 5.91 | 98.4 |
| 16 | B | 7.15 | 100.9 | 7.2 | 115.3 |
| 17 | B | 7.19 | 100.3 | 6.41 | 96.2 |
| 18 | B | 6.38 | 97.9 | <5 | 12.4 |
| 19 | B | 8.16 | 100.2 | 5.86 | 73.3 |
| 20 | B | 8.17 | 100.6 | 8.2 | 111.4 |
| 21 | B | 7.2 | 100.9 | | |
| 22 | B | 6.67 | 99.8 | | |
| 23 | B | 6.80 | 100.8 | | |
| 24 | B | 8.26 | 100.6 | 7.49 | 110.6 |
| 25 | B | 8.74 | 101.4 | 8.01 | 97.8 |
| 26 | B | 7.57 | 99.5 | 6.28 | 72.3 |
| 27 | B | 8.61 | 99.6 | 8.28 | 117.8 |
| 28 | B | 8.98 | 101.7 | 8.35 | 105.7 |
| 29 | B | 8.06 | 101.6 | 7.25 | 108.3 |
| 30 | B | 8.33 | 123.6 | 7.88 | 99.2 |
| 31 | B | 8.49 | 101.5 | 7.7 | 90.2 |
| 32 | B | 7.87 | 101.1 | | |
| 33 | B | 7.14 | 99.9 | <6 | 33.8 |
| 34 | B | 6.95 | 101.7 | | |
| 35 | B | 7.9 | 100.6 | 7.11 | 91.9 |
| 36 | B | 7.25 | 99 | | |
| 37 | B | 8.51 | 99.1 | 7.74 | 87.8 |
| 38 | B | 7.71 | 99.7 | 7.40 | 90.3 |
| 39 | B | 7.22 | 99.2 | | |
| 40 | B | 8.11 | 99.7 | | |
| 41 | B | 8.07 | 99.6 | 7.68 | 90.7 |
| 42 | B | 8.08 | 100.9 | | |
| 43 | B | 8.04 | 102.1 | 7.10 | 87 |
| 44 | B | 7.32 | 98.8 | 6.72 | 81 |
| 45 | B | 7.57 | 101.3 | 6.66 | 80.1 |
| 46 | B | 7.66 | 100.9 | 7.32 | 85.9 |
| 47 | B | 7.39 | 96.9 | 6.54 | 61.6 |
| 48 | B | 7.92 | 100.2 | 7.18 | 83 |
| 49 | B | 6.98 | 97.9 | 7.07 | 89.6 |
| 50 | B | 7.11 | 99.4 | 7.26 | 87.4 |
| 51 | B | 7.27 | 98.3 | 6.91 | 93.5 |
| 52 | B | 7.83 | 100.3 | 7.04 | 95.4 |
| 53 | B | 7.05 | 99.4 | 6.76 | 85.6 |
| 54 | B | 7.54 | 100.4 | 6.63 | 75.9 |
| 55 | B | 7.26 | 99.8 | | |
| 56 | B | 7.64 | 101.4 | 7.19 | 90.3 |
| 57 | B | 7.34 | 100.3 | | |
| 58 | B | 8.50 | 100.7 | 7.39 | 90.6 |
| 59 | B | 7.59 | 101.4 | 6.84 | 80.3 |
| 60 | B | 7.30 | 101.3 | 7.03 | 87.4 |
| 61 | B | 7.71 | 101.2 | 7.22 | 96.3 |
| 62 | B | 7.93 | 102.4 | 7.20 | 81.8 |
| 63 | B | 7.90 | 101.9 | 7.46 | 90.1 |
| 64 | B | 7.34 | 102.9 | 7.04 | 89.3 |
| 65 | B | 7.28 | 100 | | |
| 66 | B | 8.53 | 101 | 7.43 | 100.6 |
| 67 | B | 7.70 | 100.9 | 6.92 | 84.5 |
| 68 | B | 7.10 | 101.5 | | |
| 69 | B | 8.21 | 102.7 | 7.13 | 100.8 |
| 70 | B | 7.18 | 101.9 | 6.89 | 97.7 |
| 71 | B | 7.92 | 99.6 | 7.44 | 94.8 |
| 72 | B | 8.10 | 102.3 | 7.49 | 109.2 |
| 73 | B | 7.81 | 101.4 | 6.95 | 94 |
| 74 | B | 8.01 | 101 | 7.34 | 115.8 |
| 75 | B | 7.63 | 99.3 | 7.11 | 104.1 |
| 76 | B | 7.59 | 102.4 | 7.56 | 92.3 |
| 77 | B | 7.91 | 101.8 | 7.27 | 91.1 |
| 78 | B | <5 | 8.78 | | |
| 79 | B | <5 | 6.69 | | |
| 80 | B | 8.24 | 101.5 | 7.94 | 90 |
| 81 | B | 7.14 | 102.3 | 6.3 | 51.7 |
| 82 | B | 5.91 | 87.4 | | |
| 83 | B | 5.79 | 87.8 | | |
| 84 | B | 8.30 | 102.6 | 7.43 | 81.9 |
| 85 | B | 8.06 | 101.3 | 7.33 | 86.9 |
| 86 | B | 8.49 | 101.3 | 7.4 | 83.5 |
| 87 | B | 7.23 | 100.6 | <6 | 28.6 |
| 88 | B | 7.04 | 100.9 | 6.22 | 50.7 |
| 89 | B | 7.58 | 100.2 | 7.09 | 72.4 |
| 90 | B | 7.77 | 100 | 6.83 | 73.7 |
| 91 | B | 8.21 | 102 | 7.28 | 102.1 |
| 92 | B | 7.65 | 99.7 | 7.09 | 92.6 |
| 93 | B | 6.51 | 98.4 | <6 | 37.7 |
| 94 | B | <5 | 30.7 | | |
| 95 | B | 6.81 | 101 | <6 | 38.2 |
| 96 | B | 5.23 | 65.6 | | |
| 97 | B | <5 | 26 | | |
| 98 | B | <5 | 11.7 | | |
| 99 | B | <5 | −6.5 | | |
| 100 | B | 6.37 | 97 | <6 | 23.1 |
| 101 | B | 5.8 | 81.7 | | |
| 102 | B | 8.32 | 101.7 | 7.22 | 108.9 |
| 103 | B | 7.33 | 102.6 | 6.88 | 86.9 |
| 104 | B | 7.7 | 101.6 | 7.23 | 89.4 |
| 105 | B | 7.71 | 101.6 | 7.00 | 93.3 |
| 106 | B | 7.67 | 101.3 | 7.38 | 86.9 |
| 107 | B | 7.39 | 101.5 | 6.86 | 82.0 |
| 108 | B | 7.48 | 102.7 | 6.81 | 78.9 |
| 109 | B | 7.73 | 103.4 | 7.28 | 114.6 |
| 110 | B | 7.7 | 102.9 | 7.61 | 91.3 |
| 111 | B | 7.51 | 102.8 | 7.28 | 97.4 |
| 112 | B | 8.34 | 101.6 | 7.75 | 117.6 |
| 113 | B | 8.35 | 102.2 | 7.95 | 104.6 |
| 116 | B | 5.46 | 79.2 | | |
| 117 | B | 7.92 | 101.6 | 7.01 | 99.5 |
| 118 | B | 7.33 | 100.4 | 6.24 | 62.2 |
| 119 | B | 8.54 | 103.7 | 7.38 | 107.3 |
| 120 | B | 5.91 | 91.9 | | |
| 121 | B | 8.37 | 98.3 | 7.54 | 109.7 |
| 122 | B | 8.60 | 100.2 | 8.38 | 97.2 |
| 123 | B | 8.56 | 99.2 | 8.01 | 90.5 |
| 124 | B | 8.76 | 100 | | |
| 125 | B | 8.77 | 100.2 | | |
| 126 | B | 8.86 | 100.2 | 7.94 | 97.5 |
| 127 | B | 8.54 | 101.6 | 7.56 | 88.5 |
| 128 | B | 8.74 | 101.8 | 7.4 | 93.2 |
| 129 | B | 8.55 | 101.7 | 7.7 | 100.6 |
| 130 | B | 8.04 | 102.8 | 7.88 | 99 |
| 131 | B | 6.01 | 85.38 | | |
| 132 | B | 5.91 | 91.9 | | |
| 133 | B | 8.41 | 100.6 | 7.95 | 98.9 |
| 134 | B | 7.55 | 100.4 | 7.28 | 79.5 |
| 135 | B | 6.75 | 104.4 | 6.3 | 67.9 |
| 136 | B | 7.02 | 100.5 | 6.32 | 70.6 |
| 137 | B | 9.02 | 102.2 | 8.83 | 94.7 |
| 138 | B | 7.27 | 101.8 | 6.36 | 74.2 |
| 139 | B | 8 | 101 | 6.77 | 75 |
| 140 | B | 8.36 | 102.1 | 8.61 | 103.6 |
| 141 | B | 7.96 | 102.3 | 7.06 | 83.5 |
| 142 | B | 8.31 | 101.8 | 7.22 | 100.3 |
| 143 | B | 7.67 | 101.8 | 7.22 | 86.2 |
| 144 | B | 7.38 | 100.7 | <6 | 42.8 |
| 145 | B | 6.9 | 100.8 | <6 | 39.4 |
| 146 | B | 6 | 93.5 | | |
| 147 | B | 6.55 | 99 | <6 | 33.2 |
| 148 | B | 8.2 | 101.6 | 7.25 | 104.6 |
| 149 | B | 5.27 | 67.6 | | |
| 150 | B | <5 | 20.8 | | |
| 151 | B | <5 | 2.86 | | |
| 152 | B | 6.99 | 102.1 | <6 | 41.9 |
| 153 | B | <5 | 23.86 | | |
| 154 | B | 7.82 | 102.9 | 7.42 | 116.1 |
| 155 | B | 8.22 | 101.1 | 7.83 | 84.2 |
| 156 | B | 7.36 | 97.4 | 6.33 | 89 |
| 157 | B | 5.62 | 69 | | |
| 161 | B | <5 | 48 | | |
| 162 | B | 5.98 | 91 | | |
| 163 | B | 7.2 | 101 | | |
| 164 | B | 6.21 | 94 | | |
| 165 | B | 8.65 | 100 | | |
| 166 | B | 8.58 | 99 | | |
| 169 | B | 7.06 | 100 | | |
| 170 | B | 7.09 | 100 | | |
| 171 | B | 5.02 | 52 | | |
| 174 | B | <5 | 40.9 | | |

1. A compound of Formula (I′) 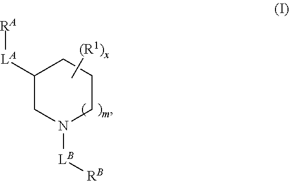
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and
C₁₋₄alkyloxy optionally substituted with 1, 2, or 3 independently selected halo substituents;
wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, >O, >CH₂, —OCH₂—, —CH₂O—, >NH, and >NCH₃;
m represents 0 or 1;
x represents 0, 1 or 2;
each R¹, when present, is bound to any available carbon atom and is independently selected from the group consisting of halo and C₁₋₄alkyl optionally substituted with 1, 2, or 3 independently selected halo substituents; or two R¹ substituents are bound to the same carbon atom and form together a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²: 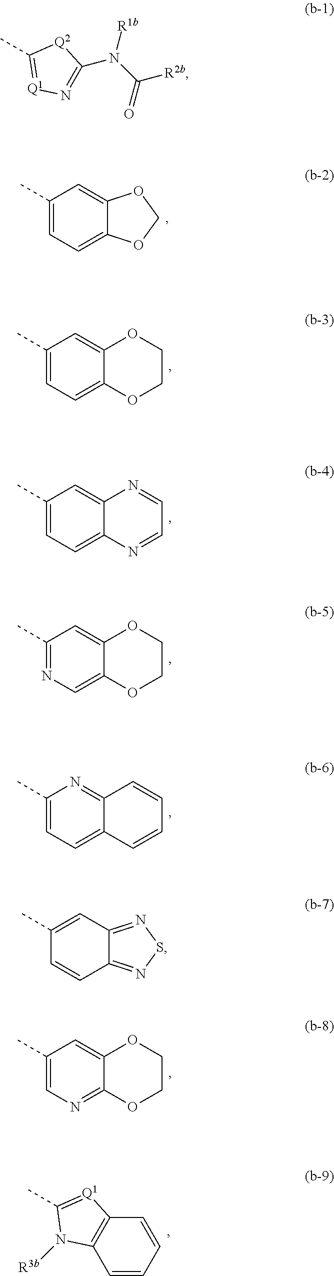 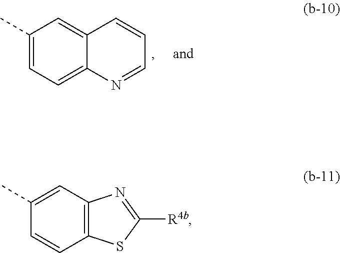
wherein
each Q¹ is CH or N;
Q² is O, NR^{q} or S;
R^{1b} is H or C₁₋₄alkyl;
R^{2b} is C₁₋₄alkyl;
R^{3b}, R^{4b}, and R^{q} are each H or C₁₋₄alkyl;
or -L^{B}-R^{B} is (b-12) 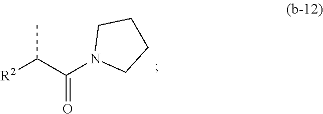
or a pharmaceutically acceptable addition salt or a solvate thereof for use as a medicament, in particular for use in treating a disorder mediated by the inhibition of O-GlcNAc hydrolase (OGA).

2. (canceled)

3. A compound of Formula (I) 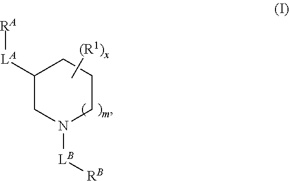
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents
L^{A} is selected from the group consisting of a covalent bond, >O, >CH₂, —OCH₂—, —CH₂O—, >NH, and >NCH₃;
m represents 0 or 1;
x represents 0, 1 or 2;
each R¹, when present, is bound to any available carbon atom and is independently selected from the group consisting of halo and C₁₋₄alkyl optionally substituted with 1, 2, or 3 independently selected halo substituents; or two R¹ substituents are bound to the same carbon atom and form together a cyclopropylidene radical;
L^{B} is selected from the group consisting of >CHR² and >SO₂;
wherein R² is selected from the group consisting of hydrogen, and C₁₋₄alkyl optionally substituted with 1, 2 or 3 independently selected halo substituents; and
R^{B} is (b-1) when L^{B} is >SO₂, or R^{B} is a radical selected from the group consisting of (b-1), (b-2), (b-3), (b-4), (b-5), (b-6), (b-7), (b-8), (b-9), (b-10), and (b-11) when L^{B} is >CHR²: 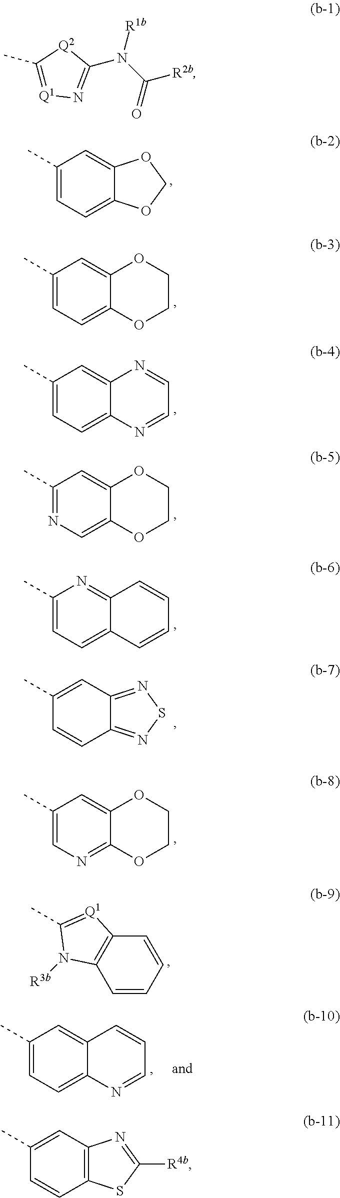
wherein
each Q¹ is CH or N;
Q² is O, NR^{q} or S;
R^{1b} is H or C₁₋₄alkyl;
R^{2b} is C₁₋₄alkyl;
R^{3b}, R^{4b}, and R^{q} are each H or C₁₋₄alkyl;
or -L^{B}-R^{B} is (b-12) 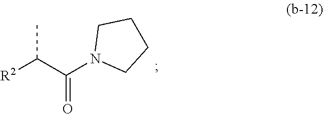
with the proviso that the compound is not
2-[1-[(2,3-dihydro-1,4-benzodioxin-6-yl)methyl]-3-piperidinyl]-pyrazine;
2-[1-[(2,3-dihydro-1,4-benzodioxin-6-yl)methyl]-3-piperidinyl]-6-methyl-pyrazine;
2-[1-[(2,3-dihydro-1,4-benzodioxin-6-yl)methyl]-3-pyrrolidinyl]-4,6-dimethyl-pyrimidine;
2-[1-[(2,3-dihydro-1,4-benzodioxin-6-yl)methyl]-3-pyrrolidinyl]-4-methyl-pyrimidine;
2-[1-(1,3-benzodioxol-5-ylmethyl)-3-piperidinyl]-pyrazine;
6-[[3-(4,6-dimethyl-2-pyrimidinyl)-1-pyrrolidinyl]methyl]-quinoline;
2-[[[1-[(2,3-dihydro-1,4-benzodioxin-6-yl)methyl]-3-piperidinyl]oxy]methyl]-pyridine;
1-methyl-2-[[3-(4-pyrimidinyl)-1-piperidinyl]methyl]-1H-benzimidazole;
1-methyl-2-[[3-(4-methyl-2-pyrimidinyl)-1-pyrrolidinyl]methyl]-1H-benzimidazole;
1-ethyl-2-[[3-(4-pyridinyloxy)-1-pyrrolidinyl]methyl]-1H-benzimidazole;
1-methyl-2-[[3-(2-pyrazinyl)-1-piperidinyl]methyl]-1H-benzimidazole;
1-methyl-2-[[3-(6-methyl-2-pyrazinyl)-1-piperidinyl]methyl]-1H-benzimidazole;
2-[[3-(4-pyrimidinyl)-1-piperidinyl]methyl]-1H-benzimidazole;
2-[[3-(4,6-dimethyl-2-pyrimidinyl)-1-pyrrolidinyl]methyl]-1-methyl-1H-benzimidazole;
1-methyl-2-[[3-(3-pyridinylmethoxy)-1-piperidinyl]methyl]-1H-benzimidazole;
2-[3-(2-pyrazinyl)-1-piperidinyl]-1-(1-pyrrolidinyl)-ethanone;
2-[3-(3-pyridinylmethyl)-1-piperidinyl]-1-(1-pyrrolidinyl)-ethanone;
2-[3-(4-methylpyrimidin-2-yl)pyrrolidin-1-yl]-1-pyrrolidin-1-yl-ethanone; or
5-[[3-(3-pyridinylmethoxy)-1-piperidinyl]methyl]-2,1,3-benzothiadiazole;
or a pharmaceutically acceptable addition salt or a solvate thereof.

4. The compound according to claim 3, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; and
C₁₋₄alkyloxy optionally substituted with 1, 2, or 3 independently selected halo substituents;
L^{A} is selected from the group consisting of a covalent bond, >O, >CH₂, —OCH₂—, —CH₂O—, >NH, and >NCH₃;
m represents 0 or 1;
x represents 0, 1 or 2; and
each R¹, when present, is bound to any available carbon atom and is independently selected from the group consisting of halo and C₁₋₄alkyl optionally substituted with 1, 2, or 3 independently selected halo substituents.

5. The compound according to claim 3, wherein
R^{A} is selected from the group consisting of pyridin-2-yl, pyridin-3-yl, pyridin-4-yl, pyridazin-3-yl, pyrimidin-4-yl, pyrimidin-5-yl, and
pyrazin-2-yl, each of which may be optionally substituted with 1, 2 or 3 substituents each independently selected from the group consisting of fluoro; cyano; C₁₋₄alkyl optionally substituted with 1, 2, or 3 independently selected fluoro substituents; and
C₁₋₄alkyloxy optionally substituted with 1, 2, or 3 independently selected fluoro substituents.

6. The compound according to claim 3, wherein R^{B} is (b-1), (b-2), (b-3), (b-4), (b-9) or (b-11).

7. The compound of Formula (I) according to claim 3, having the Formula (I-A) 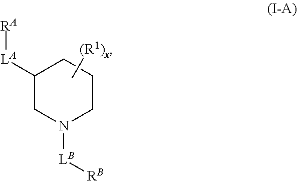
wherein all variables are as defined in any one of claims 3 to 6.

8. The compound of Formula (I) according to claim 3, having the Formula (I-B) 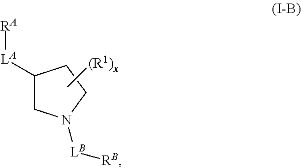
wherein all variables are as defined in any one of claims 3 to 6.

9. The compound according to claim 3, wherein R^{A} is selected from the group consisting of

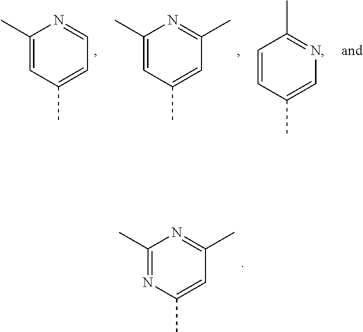

10. A pharmaceutical composition comprising a prophylactically or a therapeutically effective amount of a compound according to claim 3 and a pharmaceutically acceptable carrier.

11. A process for preparing a pharmaceutical composition comprising mixing a pharmaceutically acceptable carrier with a prophylactically or a therapeutically effective amount of a compound according to claim 3.

12. (canceled)

13. (canceled)

14. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 3.

15. A method for inhibiting O-GlcNAc hydrolase, comprising administering to a subject in need thereof, a prophylactically or a therapeutically effective amount of a compound according to claim 3.
